SEQUENCE LISTING (38 sequences)

SEQ ID NO: 1 - protein, 8 aa, Artificial Sequence
  VH_CD40-001_CDR1
GYTFTEYI

SEQ ID NO: 2 - protein, 8 aa, Artificial Sequence
  VH_CD40-001_CDR2
IIPNNGGT

SEQ ID NO: 3 - protein, 15 aa, Artificial Sequence
  VH_CD40-001_CDR3
TRREVYGRNYYALDY

SEQ ID NO: 4 - protein, 6 aa, Artificial Sequence
  VL_CD40-001_CDR1
QGINNY

SEQ ID NO: 5 - protein, 3 aa, Artificial Sequence
  VL_CD40-001_CDR2
YTS

SEQ ID NO: 6 - protein, 9 aa, Artificial Sequence
  VL_CD40-001_CDR3
QQYSNLPYT

SEQ ID NO: 7 - protein, 122 aa, Artificial Sequence
  VH_CD40-001
EVQLQQSGPDLVKPGASVKISCKTSGYTFTEYIMHWVKQSHGKSLEWIGGIIPNNGGTSYNQKFKDKATMTVDKSSSTGYMELRSLTSEDSAVYYCTRREVYGRNYYALDYWGQGTLVTVSS

SEQ ID NO: 8 - protein, 107 aa, Artificial Sequence
  VL_CD40-001
DIQMTQTTSSLSASLGDRVTITCSASQGINNYLNWYQQKPDGTVKLLIYYTSSLHSGVPSRFSGSGSGTDYSLTISNLEPEDIATYYCQQYSNLPYTFGGGTKLEIK

SEQ ID NO: 9 - protein, 122 aa, Artificial Sequence
  VH_CD40-001 humanized
EVQLVQSGAEVKKPGASVKVSCKTSGYTFTEYIMHWVRQAPGQGLEWMGGIIPNNGGTSYNQKFQGRVTMTVDKSTSTGYMELSSLRSEDTAVYYCTRREVYGRNYYALDYWGQGTLVTVSS

SEQ ID NO: 10 - protein, 107 aa, Artificial Sequence
  VL_CD40-001 humanized
DIQMTQSPSSLSASVGDRVTITCSASQGINNYLNWYQQKPGKAVKLLIYYTSSLHSGVPSRFSGSGSGTDYTFTISSLQPEDIATYYCQQYSNLPYTFGGGTKVEIK

SEQ ID NO: 11 - protein, 8 aa, Artificial Sequence
  VH_CD137-009_CDR1
GFSLNDYW

SEQ ID NO: 12 - protein, 7 aa, Artificial Sequence
  VH_CD137-009_CDR2
IDVGGSL

SEQ ID NO: 13 - protein, 11 aa, Artificial Sequence
  VH_CD137-009_CDR3
ARGGLTYGFDL

SEQ ID NO: 14 - protein, 6 aa, Artificial Sequence
  VL_CD137-009_CDR1
EDISSY

SEQ ID NO: 15 - protein, 3 aa, Artificial Sequence
  VL_CD137-009_CDR2
GAS

SEQ ID NO: 16 - protein, 12 aa, Artificial Sequence
  VL_CD137-009_CDR3
HYYATISGLGVA

SEQ ID NO: 17 - protein, 114 aa, Artificial Sequence
  VH_CD137-009
QSLEESGGRLVTPGTPLTLTCTVSGFSLNDYWMSWVRQAPGKGLEWIGYIDVGGSLYYASWAKGRFTISRTSTTVDLKMTSLTTEDTATYFCARGGLTYGFDLWGPGTLVTVSS

SEQ ID NO: 18 - protein, 110 aa, Artificial Sequence
  VL_CD137-009
DIVMTQTPASVSEPVGGTVTINCQASEDISSYLAWYQQKPGQRPKRLIYGASDLASGVPSRFSASGSGTEYALTISDLESADAATYYCHYYATISGLGVAFGGGTEVVVK

SEQ ID NO: 19 - protein, 117 aa, Artificial Sequence
  VH_CD137-009 humanized
EVQLVESGGGLVQPGRSLRLSCTASGFSLNDYWMSWVRQAPGKGLEWVGYIDVGGSLYYAASVKGRFTISRDDSKSIAYLQMNSLKTEDTAVYYCARGGLTYGFDLWGQGTLVTVSS

SEQ ID NO: 20 - protein, 110 aa, Artificial Sequence
  VL_CD137-009 humanized
DIVMTQSPSSLSASVGDRVTITCQASEDISSYLAWYQQKPGKAPKRLIYGASDLASGVPSRFSASGSGTDYTFTISSLQPEDIATYYCHYYATISGLGVAFGGGTKVEIK

SEQ ID NO: 21 - protein, 330 aa, Artificial Sequence
  IgG1-Fc
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 330 aa, Artificial Sequence
  IgG1-Fc_F405L
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 23 - protein, 330 aa, Artificial Sequence
  IgG1-Fc_K409R
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 24 - protein, 330 aa, Artificial Sequence
  IgG1-Fc_FEA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 330 aa, Artificial Sequence
  IgG1-FEAL-Fc
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 26 - protein, 330 aa, Artificial Sequence
  IgG1-FEAR-Fc
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 27 - protein, 107 aa, Artificial Sequence [sequence omitted]

SEQ ID NO: 28 - protein, 106 aa, Artificial Sequence
  Lambda-C
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 29 - protein, 329 aa, Artificial Sequence
  IgG1-Fc without C-terminal Lys
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 329 aa, Artificial Sequence
  IgG1-Fc_F405L without C-terminal Lys
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 31 - protein, 329 aa, Artificial Sequence
  IgG1-Fc_K409R without C-terminal Lys
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 32 - protein, 329 aa, Artificial Sequence
  IgG1-Fc_FEA without C-terminal Lys
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 329 aa, Artificial Sequence
  IgG1-FEAL-Fc without C-terminal Lys
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 34 - protein, 329 aa, Artificial Sequence
  IgG1-FEAR-Fc without C-terminal Lys
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 35 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 36 - protein, 257 aa, Homo sapiens
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 37 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 38 - protein, 232 aa, Homo sapiens
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

(19) United States
(12) Patent Application Publication
(10) Pub. No.: US 2024/0174759 A1
(43) Pub. Date: May 30, 2024

(54) MULTISPECIFIC BINDING AGENTS AGAINST CD40 AND CD137 IN THERAPY

(71) Applicants: GENMAB A/S, Valby (DK); BIONTECH SE, Mainz (DE)

(72) Inventors: Yali FU, Plainsboro, NJ (US); Homer ADAMS, Plainsboro, NJ (US); Gaurav BAJAJ, Plainsboro, NJ (US)

(21) Appl. No.: 18/549,346

(22) PCT Filed: Mar. 9, 2022
(86) PCT No.: PCT/EP2022/056021

§ 371 Date: Sep. 6, 2023

Related U.S. Application Data
(60) Provisional application No. 63/158,633, filed on Mar. 9, 2021.

(51) Int. Cl. C07K 16/28; A61K 9/00; A61K 39/00; A61P 35/00

(52) U.S. Cl. CPC C07K 16/2878; A61K 9/0019; A61P 35/00; A61K 2039/505; A61K 2039/54; A61K 2039/545; C07K 2317/31; C07K 2317/524; C07K 2317/526; C07K 2317/565

(57) ABSTRACT

The present disclosure relates generally to the field of multispecific binding agents for use in therapy, in particular for use in treating cancer, wherein the binding agents bind to human CD40 and to human CD137.

### TECHNICAL FIELD

The present disclosure relates generally to the field of multispecific binding agents for use in therapy, in particular for use in treating cancer, wherein the binding agents bind to human CD40 and to human CD137.

### BACKGROUND

CD40 is a member of the tumor necrosis factor (TNF) receptor (TNFR) family and is known as a co-stimulatory protein found on a diversity of cell types. CD40 is constitutively expressed by antigen-presenting cells (APCs), including dendritic cells (DCs), B cells and macrophages. It can also be expressed by endothelial cells, platelets, smooth muscle cells, fibroblasts and epithelial cells. Consistent with its widespread expression on normal cells, CD40 is also expressed on a wide range of tumor cells.

The presentation of peptide antigens in the context of MHC class II molecules to antigen-specific CD4⁺ T cells, together with co-stimulatory signals (from CD80 and/or CD86), results in the activation of CD4⁺ T cells and the up-regulation of the DC licensing factors CD40 ligand (CD40L) and lymphotoxin-α1β2 (LTα1β2). Expression of CD40L and LT LTα1β2 on activated antigen-specific CD4⁺ T cells induces signaling through CD40 and the LTD receptor (LTOR), and this licenses DCs to induce CD8+ T-cell responses. CD40 signaling results in the production of interleukin-12 (IL-12) and the up-regulation of CD70, CD86, 4-1BB ligand (4-1BBL), OX40 ligand (OX40L) and GITR ligand (GITRL), whereas LTOR signaling leads to the production of type I interferons (IFNs). The signaling system that controls the activity of nuclear factor kappaB (NF-κB) is responsive to virtually all TNFR superfamily members. Pathogen-associated molecular patterns (PAMPs) and damage-associated molecular patterns (DAMPs) also contribute to these events. Priming of CD8⁺ T cells by MHC class I-restricted peptides results in the up-regulation of CD27, 4-1BB, OX40 and glucocorticoid-induced TNFR-related protein (GITR). Stimulation of these receptors on CD8⁺ T cells by their cognate TNF superfamily ligands, in combination with IL-12 and type I IFNs, results in robust CD8⁺ T cell activation, proliferation and effector function, as well as the formation and maintenance of CD8⁺ T cell memory. CD40 antibodies can exert different actions, such as CD40-expressing tumor cell kill by induction of antibody-dependent cell-mediated cytotoxicity (ADCC), complement-dependent cytotoxicity (CDC) or antibody-dependent cell-mediated phagocytosis (ADCP), induction of cell signaling to induce direct apoptosis or growth arrest, but also, independent of CD40 expression on the tumor cells, through licensing of APCs to stimulate an anti-cancer immune response. Antibodies binding to CD40 can trigger CD40 on APCs to prime effector cytotoxic T lymphocytes (CTLs) and induce release of IL-2 by these cells, and indirectly activate NK cells. Antibodies stimulating CD40 have been disclosed in the prior art, and include CP-870,893, a human IgG2 antibody (WO 03/040170); dacetuzumab, a humanized IgG1 antibody (WO 00/075348) and Chi Lob 7/4, a chimeric IgG1 antibody (US 2009/0074711). Furthermore, an antagonistic CD40 antibody has been disclosed, lucatumumab, a human IgG1 antibody (WO 02/028481). CD137 (4-1BB) is also a member of the TNFR family. CD137 is a co-stimulatory molecule on CD8^{m} and CD4⁺ T cells, regulatory T cells (Tregs), Natural Killer T cells (NK(T) cells), B cells and neutrophils. On T cells, CD137 is not constitutively expressed, but induced upon T-cell receptor (TCR) activation (for example, on tumor infiltrating lymphocytes (TILs) (Gros et al., J. Clin Invest 2014; 124(5):2246-59)). Stimulation via its natural ligand 4-1BBL or agonist antibodies leads to signaling using TRAF-2 and TRAF-1 as adaptors. Early signaling by CD137 involves K-63 poly-ubiquitination reactions that ultimately result in activation of the nuclear factor (NF)-κB and mitogen-activated protein (MAP)-kinase pathways. Signaling leads to increased T cell co-stimulation, proliferation, cytokine production, maturation and prolonged CD8+ T-cell survival. Agonistic antibodies against CD137 have been shown to promote anti-tumor control by T cells in various pre-clinical models (Murillo et al., Clin Cancer Res 2008; 14(21):6895-906). Antibodies stimulating CD137 can induce survival and proliferation of T cells, thereby enhancing the anti-tumor immune response. Antibodies stimulating CD137 have been disclosed in the prior art, and include urelumab, a human IgG4 antibody (AU 2004279877) and utomilumab, a human IgG2 antibody (Fisher et al., 2012, Cancer Immunol. Immunother. 61: 1721-1733).

Westwood J A, et al., Leukemia Research 38 (2014), 948-954 discloses “Combination anti-CD137 and anti-CD40 antibody therapy in murine myc-driven hematological cancers”. WO 2018/011421 provides binding agents, such as bispecific antibodies, binding human CD40 and binding human CD137.

However, despite these advances in the art there is a considerable need for improved therapies targeting CD40 and CD137.

### SUMMARY

In a first aspect, the present disclosure provides a binding agent for use in a method for reducing or preventing progression of a tumor or treating cancer in a subject, said method comprising administering to said subject the binding agent in a suitable amount, wherein the binding agent comprises a first binding region binding to human CD40, such as human CD40 comprising the sequence set forth in SEQ ID NO: 36, and a second binding region binding to human CD137, such as human CD137 comprising the sequence set forth in SEQ ID NO: 38.

In one embodiment of the first aspect, the suitable amount of the binding agent is a therapeutically effective and safe amount. For example, the suitable amount of the binding agent is about 0.04-2.5 mg/kg body weight or about 3-200 mg in total; and/or about 0.25×10⁻⁹-16.9×10⁻⁹ mol/kg body weight or about 20×10⁻⁹-1350×10⁻⁹ mol in total.

In one embodiment of the first aspect, the binding agent is administered systemically, preferably intravenously.

In a second aspect, the present disclosure provides a composition comprising a binding agent comprising a first binding region binding to human CD40 and a second binding region binding to human CD137, wherein the amount of binding agent in the composition is between about 3-200 mg or about 20×10⁻⁹-1350×10⁻⁹ mol.

In a third aspect, the present disclosure provides a composition of the second aspect for use in a method for reducing or preventing progression of a tumor or treating cancer in a subject.

In a fourth aspect, the present disclosure provides a method for reducing or preventing progression of a tumor or treating cancer in a subject, said method comprising administering to said subject a binding agent in a suitable amount, wherein the binding agent comprises a first binding region binding to human CD40, such as human CD40 comprising the sequence set forth in SEQ ID NO: 36, and a second binding region binding to human CD137, such as human CD137 comprising the sequence set forth in SEQ ID NO: 38. In a fifth aspect, the present disclosure provides a method for reducing or preventing progression of a tumor or treating cancer in a subject, said method comprising administering to said subject a composition comprising binding agent in an amount between about 3-200 mg or about 20×10⁻⁹-1350×10⁻⁹ mol, wherein the binding agent comprises a first binding region binding to human CD40, such as human CD40 comprising the sequence set forth in SEQ ID NO: 36, and a second binding region binding to human CD137, such as human CD137 comprising the sequence set forth in SEQ ID NO: 38.

### BRIEF DESCRIPTION OF THE FIGURES

FIG. 1: Schematic representation of the anticipated mode of action

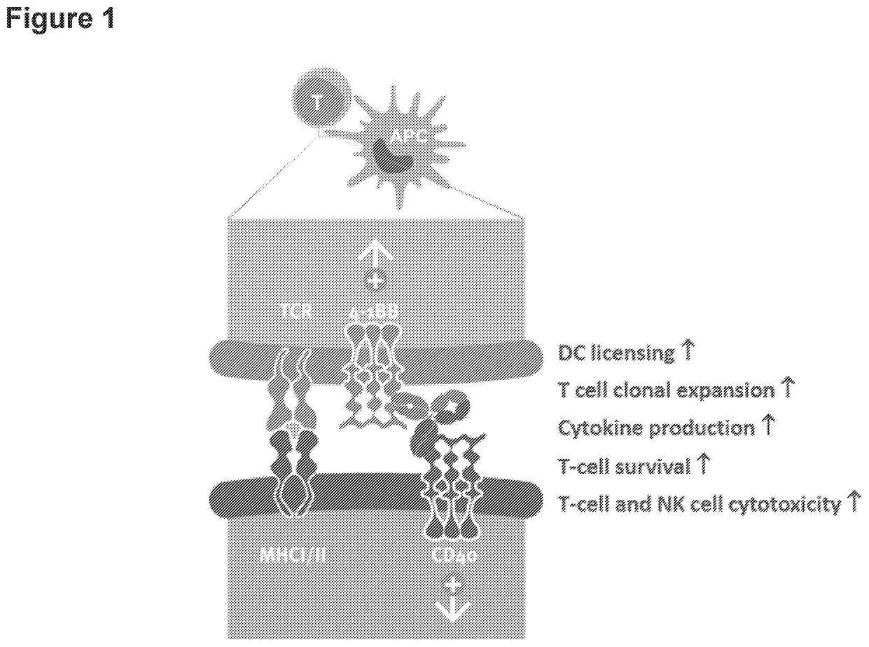

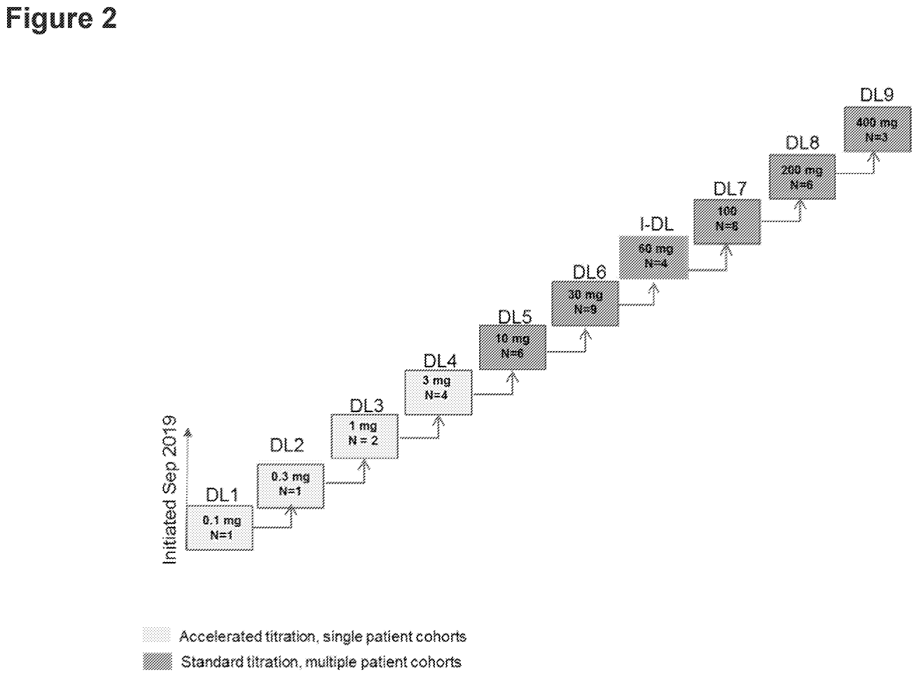

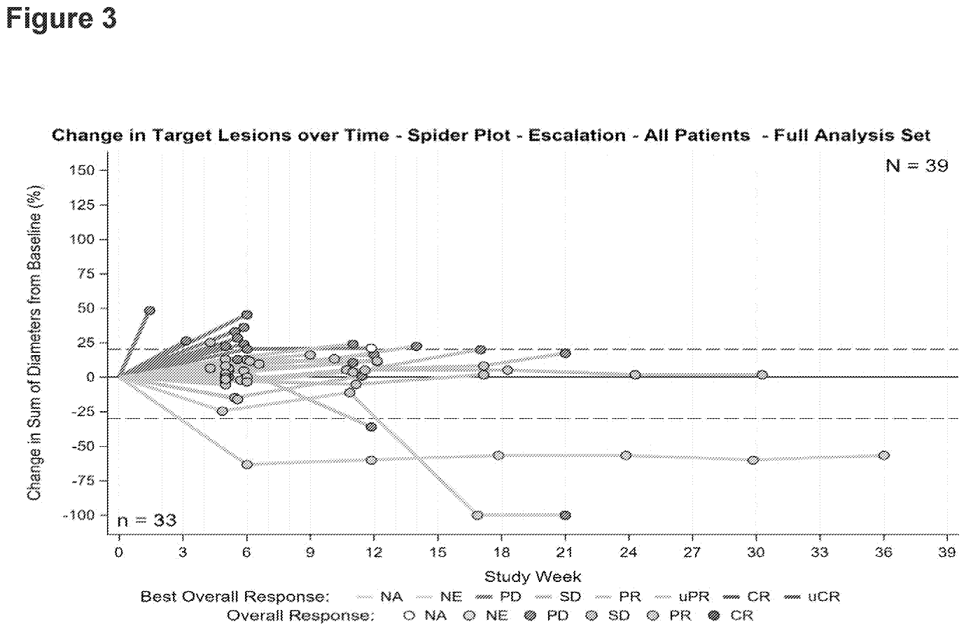

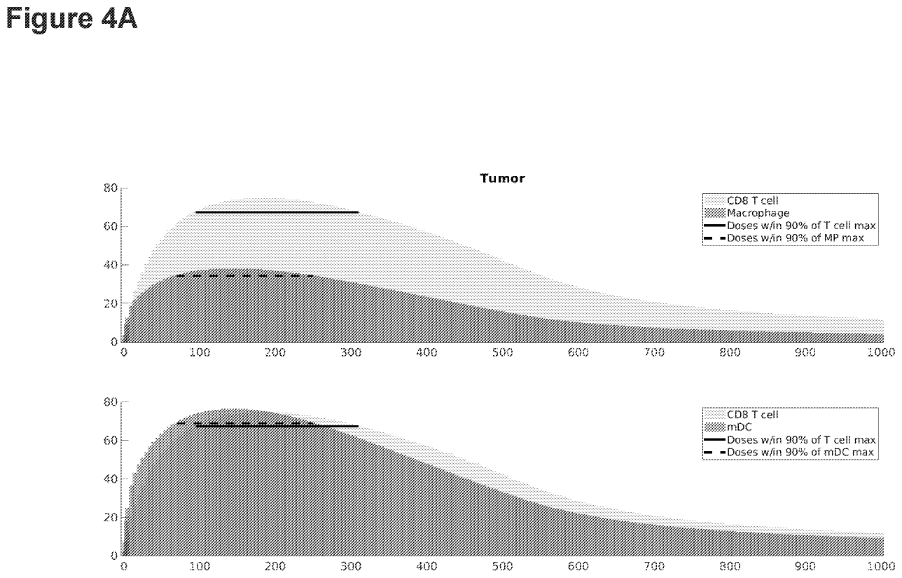

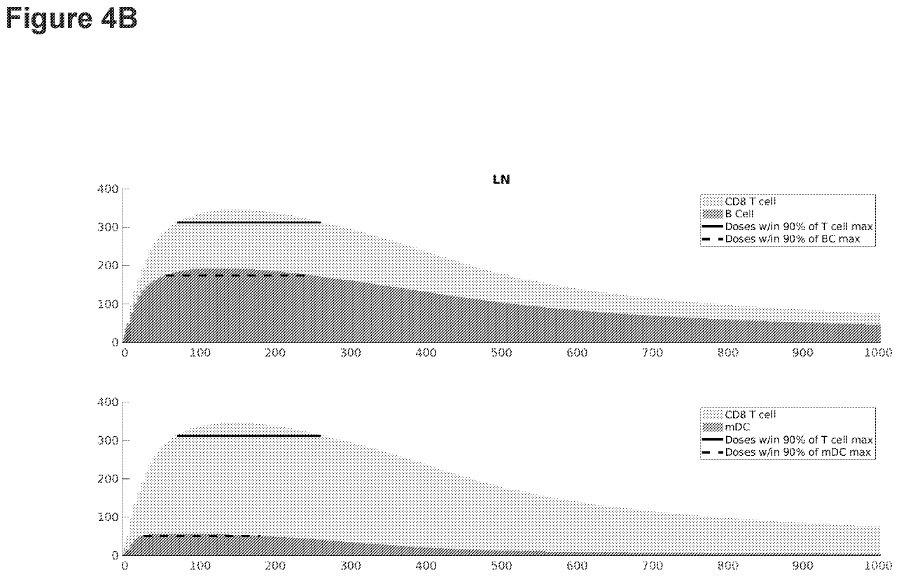

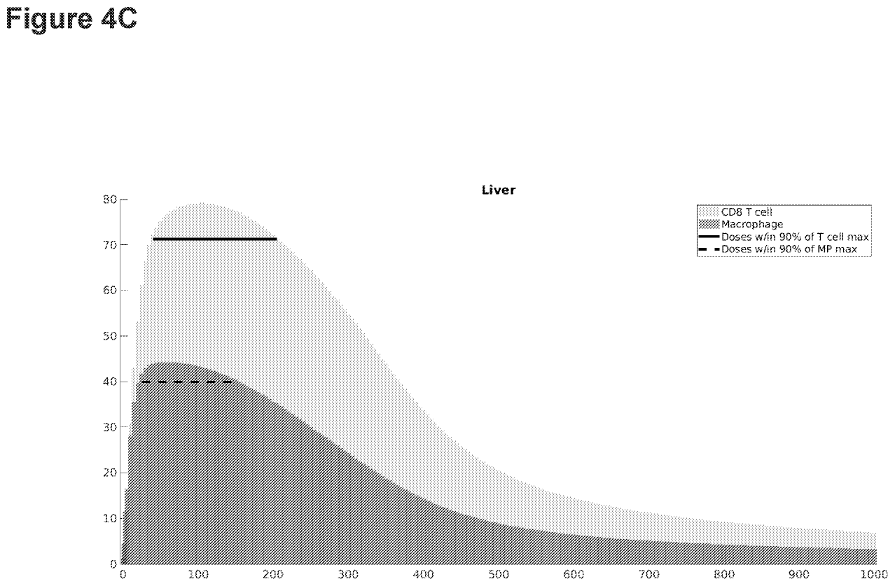

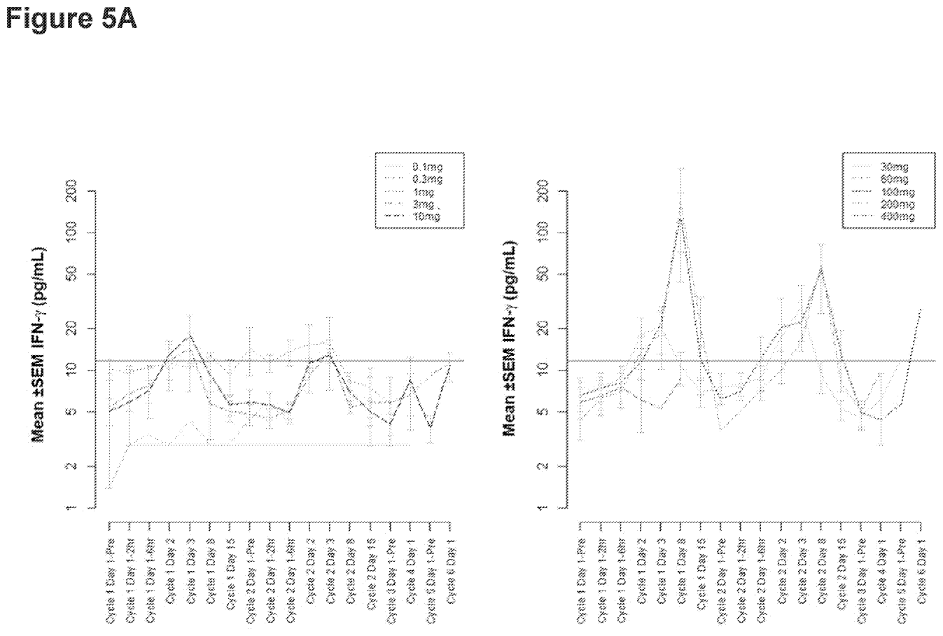

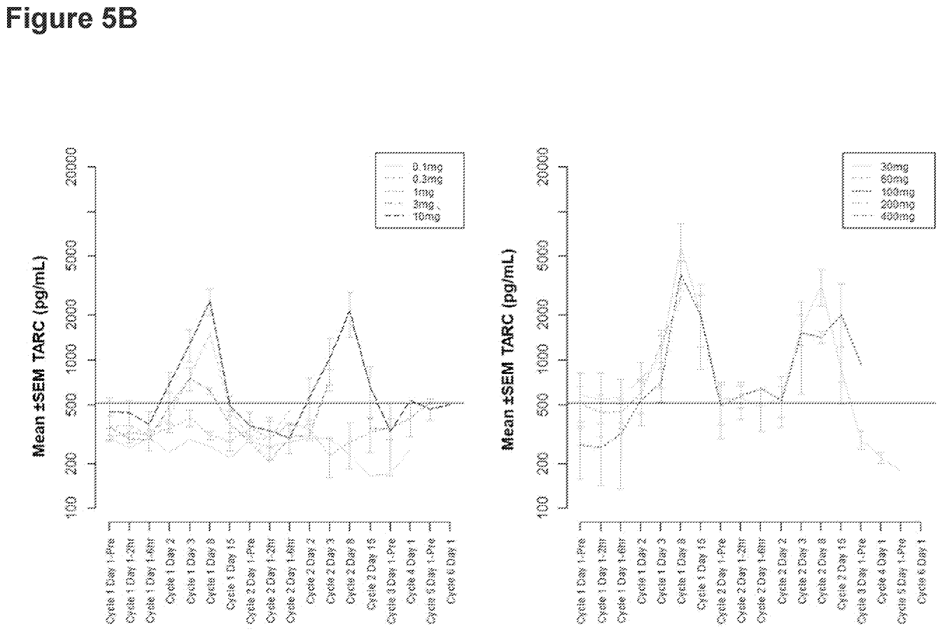

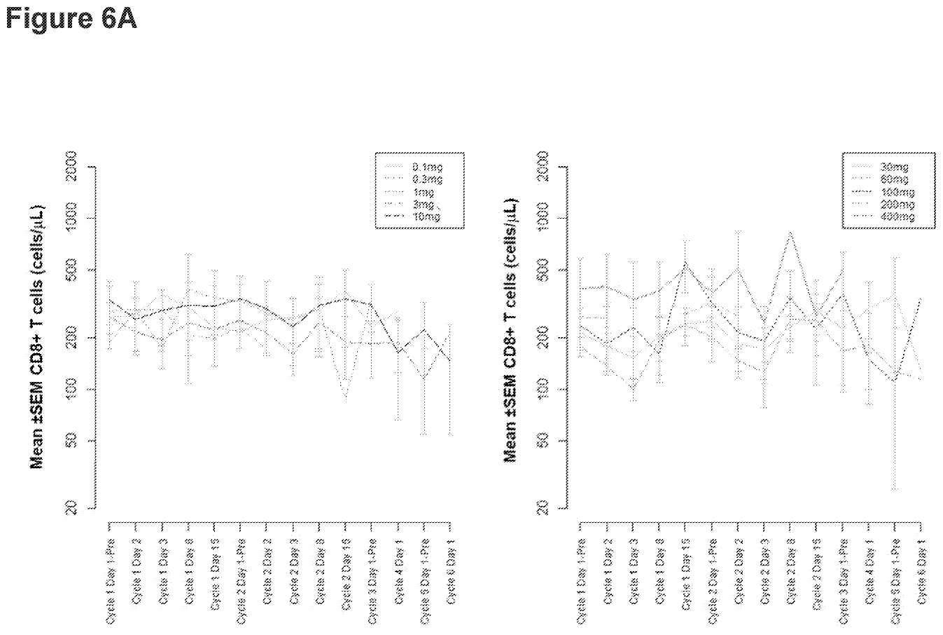

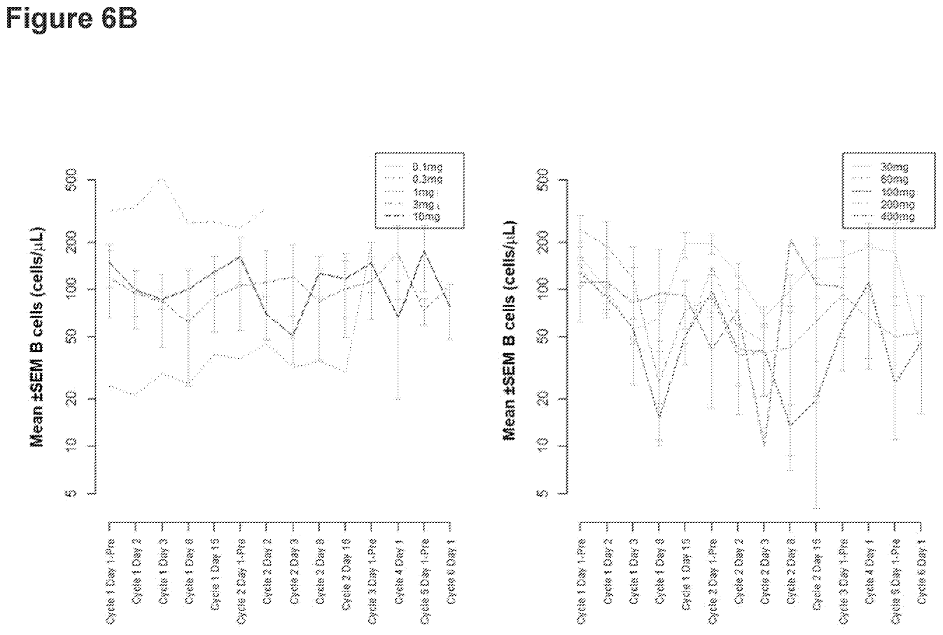

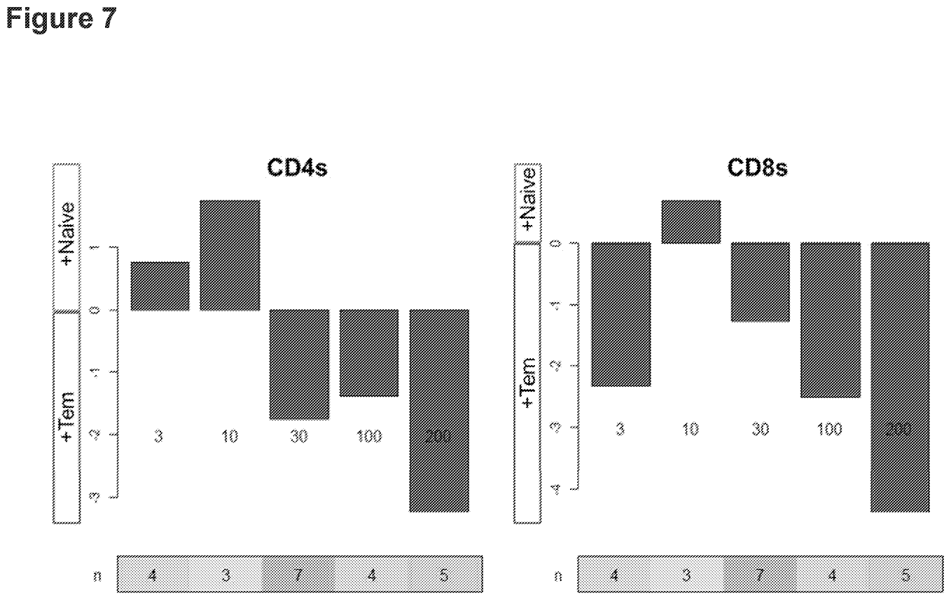

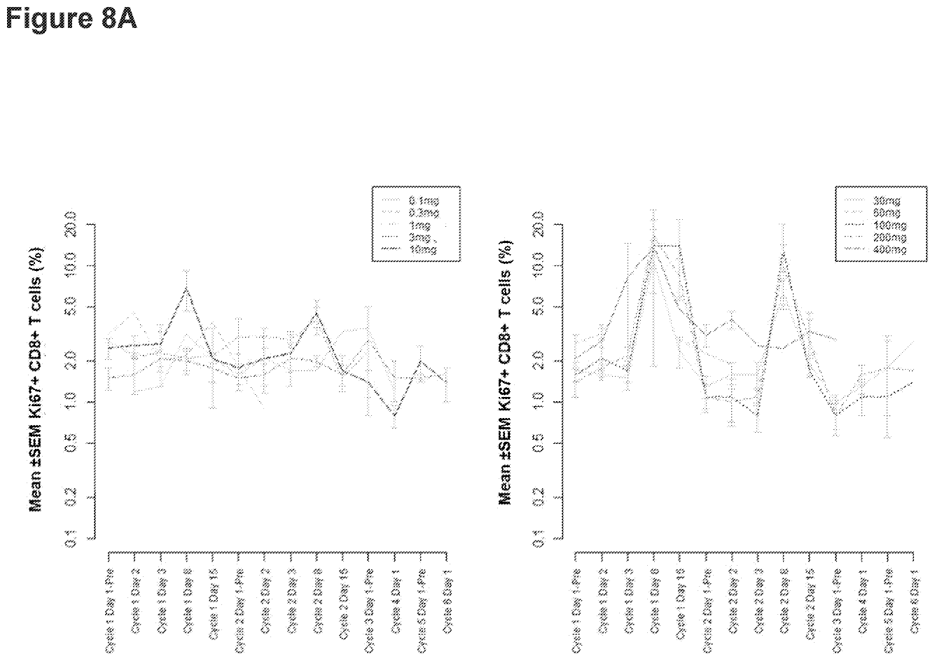

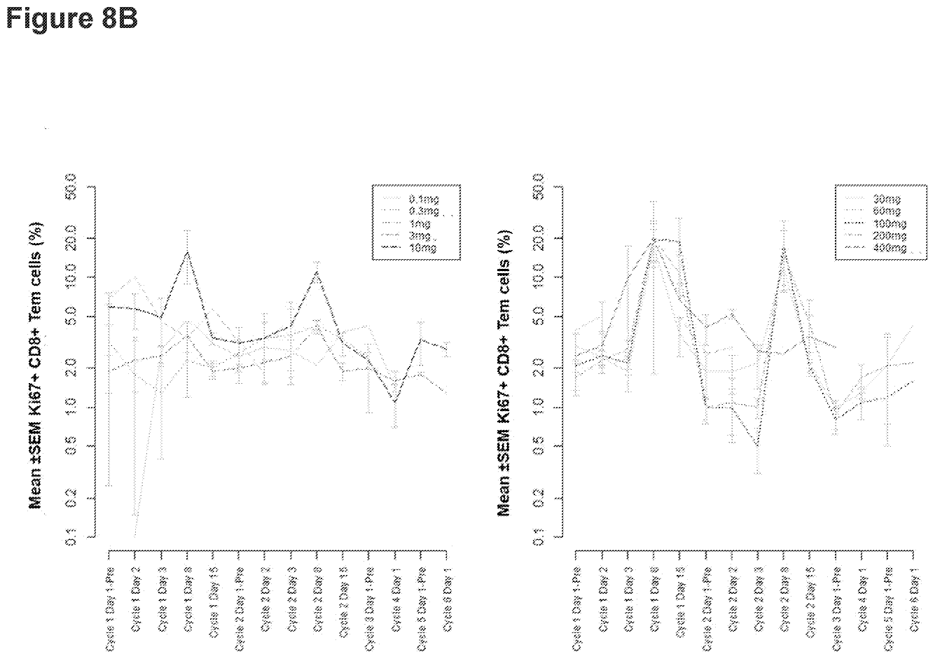

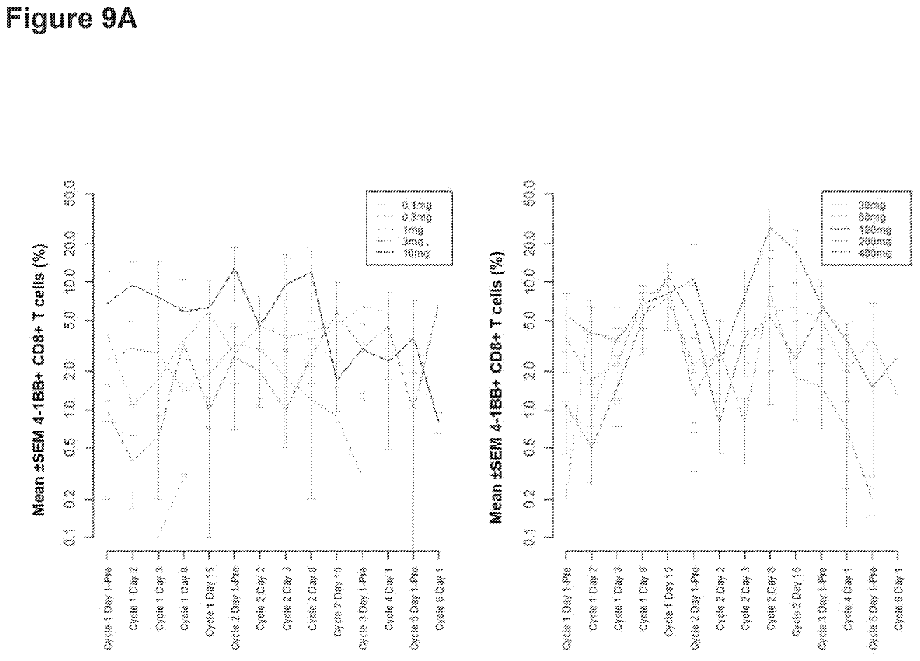

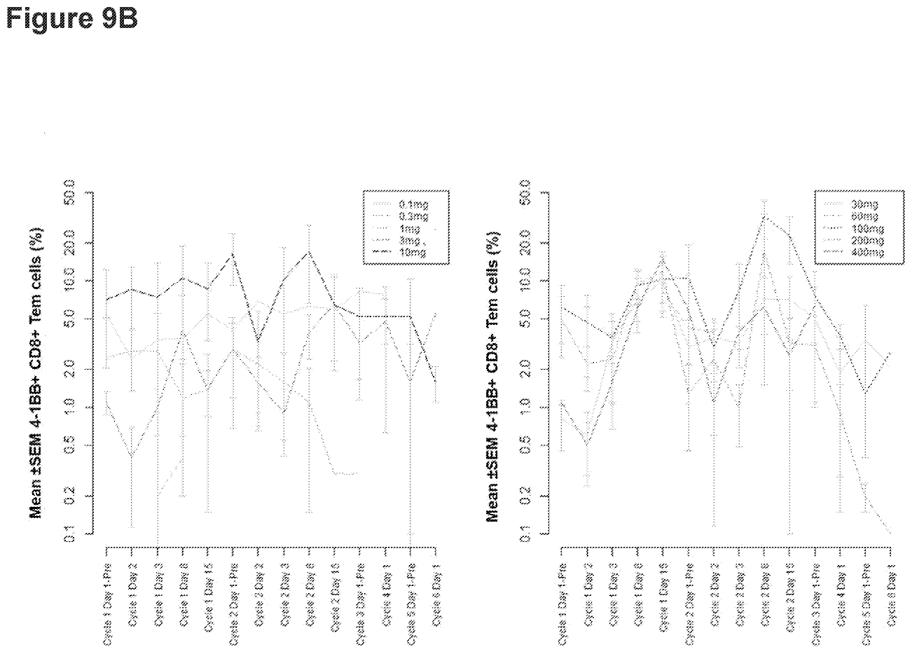

Schematic representation of the anticipated mode of action of the CD40×4-1BB bispecific antibody GEN1042. CD40 is expressed on antigen-presenting cells (APCs) as well as on tumor cells. CD137 is expressed on activated T cells. DuoBody-CD40×4-1BB (GEN1042) is a bispecific antibody that crosslinks CD40 on antigen presenting cells (APCs) with 4-1BB on activated T cells, thereby inducing conditional stimulation of and co-stimulatory activity in both cell types. Thereby CD40×4-1BB bispecific antibodies are anticipated to enhance DC licencing, T-cell clonal expansion, cytokine production, T-cell survival and T-cell and NK-cell-mediated cytotoxicity.

FIG. 2: Schematic outline of clinical trial design

Schematic representation of the Phase 1 Dose Escalation Part of the First in Human (FIH), open-label, safety trial of GEN1042 in subjects with all solid malignant tumours. Dose levels ranging from 0.1 mg to 400 mg were explored using an accelerated titration phase consisting of single-subject cohorts followed by larger cohorts informed by the modified Continuous Reassessment Method (mCRM) and Escalation with Overdose Control (EWOC) design. In the mCRM, the relationship between probability of a DLT and dose level were described by a Bayesian Logistic Regression Model (BLRM) and were used to guide dose recommendations and to estimate the Maximum Tolerated Dose (MTD).

FIG. 3: Preliminary clinical activity, change in target lesions over time—spider plot Dose escalation; best change from baseline in tumor size, patients with NSCLC. Data cut-off. January 8th, 2021. NA, not available; NE, non-evaluable; PD, progressive disease; SD, stable disease; PR, partial response; uPR, unconfirmed partial response; CR, complete response; uCR, unconfirmed complete response. Plot of the percentage change in tumour measurements overtime in 39 evaluable patients enrolled in the Phase 1 dose escalation of the GCT1042-01 trial who at least one post-baseline lesion assessment.

FIG. 4: Physiological based Pharmacokinetics Pharmacodynamic modelling

Model predicted area under the curve for percent trimer levels after 1st cycle with respect to dose given as Q3W in a) tumor, B) lymph nodes (LN), and C) liver. Blue bar indicated engagement of 4-1BB on CD8⁺ T-cells, and red (or grey overlap) indicates engagement of CD40 on APCs (macrophage, B-cells or mDCs). FIGS. 4A, 4B and 4C show AUC for predicted trimer levels with respect to dose. Maximum engagement for 4-1BB on T-cells was observed in range of 100-200 mg in tumors and LN, and around 50-200 mg in liver. Engagement for CD40 was observed at similar rage on APCs. Increasing doses >200 mg resulted in reduced trimer formation. In addition, based on available clinical pharmacodynamic data, higher magnitude, and consistent modulation of peripheral pharmacodynamic endpoints (IFNγ and proliferating Ki67+ effector memory CD8⁺ T cells) were seen at dose levels up to 200 mg. Considering, PBPK/PD modeling predictions and available clinical data, the optimal dose of GEN1042 was predicted to be in the range of 100 mg 1Q3W.

FIG. 5: INF-γ and TARC increase

Circulating levels of INF-γ (FIG. 5A) and TARC (FIG. 5B) were measured in serum samples at baseline, and at multiple time points post administration of GEN1042 in cycle 1 and cycle 2 (days 1 [pre, 2 h and between 4-6 h post-administration], 2, 3, 8, and 15) and pre-dose for cycles 3 and beyond. Interim data limits mature data at all time points, therefore maximal n available for each dose TARC were (0.1 mg=[1]; 0.3 mg=[1]; 1 mg=[2]; 3 mg=[2]; 10 mg=[6]; 30 mg=[6]; 60 mg=[0]; 100 mg=[5]; 200 mg=[3]; 400 mg=[0]). TARC levels in serum samples were determined by Meso Scale Discovery (MSD) multiplex immune assay. Abbreviations: pg=picograms, mL=milliliters, hr=hours, mg=milligrams, SEM=standard error of means, pre=pre-dose

Test reference range: TARC (pg/mL)<513

DCO (data cut off)=Jan. 22, 2021

FIG. 6: Trafficking/Margination of T and B cells

FIG. 6 shows the induction of transient trafficking/margination of CD8 T cells (FIG. 6A) and B cells (FIG. 6B) by administration of GEN1042._Immunophenotyping of peripheral blood was conducted in whole blood collected at baseline and at multiple timepoints post administration of GEN1042 in cycle 1 and cycle 2 (days 1, 2, 3, 8 and 15) and pre-dose for cycles 3 and beyond. Interim data limits mature data at all timepoints, therefore maximal n available for each dose were (0.1 mg=[1]; 0.3 mg=[1]; 1 mg=[2]; 3 mg=[4]; 10 mg=[6]; 30 mg=[9]; 60 mg=[3]; 100 mg=[6]; 200 mg=[6]; 400 mg=[3]). The frequency of CD8 T cells was assessed in whole blood samples by flow cytometry.

Abbreviations: μL=microliters, hr=hours, mg=milligrams, SEM=standard error of means, pre=pre-dose

DCO=Jan. 22, 2021

FIG. 7: T cell Maturation Expansion

Immunophenotyping of peripheral blood was conducted in whole blood collected at baseline and at multiple timepoints post administration of GEN1042 in cycle 1 and cycle 2 (days 1, 2, 3, 8 and 15) and pre-dose for cycles 3 and beyond. Interim data limits mature data at all timepoints. Area Under the Curve (AUC) values were calculated for each patient using baseline-normalized values out to cycle 2 day 15 for CD4 and CD8 naïve or effector memory (Tem) T cells. Then the average AUC was computed for patients within each dose level and the difference between naïve and Tem cells within the CD4 or CD8 populations was calculated. Values plotted in grey bars indicate these difference values between the naïve and Tem populations, while sample sizes are represented along the bottom of each plot. The frequency of naïve and effector memory T cells subsets was assessed in whole blood samples by flow cytometry.

DCO=Jan. 22, 2021

FIG. 8: T cell Proliferation

FIG. 8 shows the proliferation of total CD8+ T cells (FIG. 8A) and CD8+ effector memory T cells (FIG. 8B) as measured by an increase in the frequency of % Ki67+ populations after administration of GEN1042. Immunophenotyping of peripheral blood was conducted in whole blood collected at baseline and at multiple time points post administration of GEN1042 in cycle 1 and cycle 2 (days 1, 2, 3, 8 and 15) and pre-dose for cycles 3 and beyond. Interim data limits mature data at all time points, therefore maximal n available for each dose were (0.1 mg=[1]; 0.3 mg=[1]; 1 mg=[2]; 3 mg=[4]; 10 mg=[6]; 30 mg=[9]; 60 mg=[3]; 100 mg=[6]; 200 mg=[6]; 400 mg=[3]). The frequency of CD8 T cells and effector memory T cells subsets as well as their level of proliferation (% Ki67+) were assessed in whole blood samples by flow cytometry.

Abbreviations: SEM=standard error of means, Tem=effector memory T cells, mg=milligrams, pre=pre-dose

DCO=Jan. 22, 2021

FIG. 9: T cell Activation

FIG. 9 shows the activation of total CD8+ T cells (FIG. 9A) and CD8+ effector memory T cells (FIG. 9B) as measured by an increase in the frequency of %4-1BB+ populations after administration of GEN1042. Immunophenotyping of peripheral blood was conducted in whole blood collected at baseline and at multiple time points post administration of GEN1042 in cycle 1 and cycle 2 (days 1, 2, 3, 8 and 15) and pre-dose for cycles 3 and beyond. Interim data limits mature data at all time points, therefore maximal n available for each dose were (0.1 mg=[1]; 0.3 mg=[1]; 1 mg=[2]; 3 mg=[4]; 10 mg=[6]; 30 mg=[9]; 60 mg=[3]; 100 mg=[6]; 200 mg=[6]; 400 mg=[3]). The frequency of CD8 T cells and effector memory T cells subsets as well as their level of activation (%4-1BB+) were assessed in whole blood samples by flow cytometry. Abbreviations: SEM=standard error of means, Tem=effector memory T cells, mg=milligrams, pre=pre-dose

DCO=Jan. 22, 2021

TABLE 1
Sequences

| Name | Type | Sequence | SEQ ID |
|---|---|---|---|
| CD40-001 antibody | VH CDR1 | GYTFTEYI | 1 |
| (mouse) | | | |
| | VH CDR2 | IIPNNGGT | 2 |
| | VH CDR3 | TRREVYGRNYYALDY | 3 |
| | VL CDR1 | QGINNY | 4 |
| | VL CDR2 | YTS | 5 |
| | VL CDR3 | QQYSNLPYT | 6 |
| | VH | EVQLQQSGPDLVKPGASVKISCKTSGYTFTEYIMHWV | 7 |
| | | KQSHGKSLEWIGGIIPNNGGTSYNQKFKDKATMTVDK | |
| | | SSSTGYMELRSLTSEDSAVYYCTRREVYGRNYYALDY | |
| | | WGQGTLVTVSS | |
| | VL | DIQMTQTTSSLSASLGDRVTITCSASQGINNYLNWYQ | 8 |
| | | QKPDGTVKLLIYYTSSLHSGVPSRFSGSGSGTDYSLTI | |
| | | SNLEPEDIATYYCQQYSNLPYTFGGGTKLEIK | |
| | VH | EVQLVQSGAEVKKPGASVKVSCKTSGYTFTEYIMHWV | 9 |
| | humanized | RQAPGQGLEWMGGIIPNNGGTSYNQKFQGRVTMTVD | |
| | | KSTSTGYMELSSLRSEDTAVYYCTRREVYGRNYYALD | |
| | | YWGQGTLVTVSS | |
| | VL | DIQMTQSPSSLSASVGDRVTITCSASQGINNYLNWYQ | 10 |
| | humanized | QKPGKAVKLLIYYTSSLHSGVPSRFSGSGSGTDYTFTI | |
| | | SSLQPEDIATYYCQQYSNLPYTFGGGTKVEIK | |
| CD137 antibody | VH CDR1 | GFSLNDYW | 11 |
| clone 009 (rabbit) | | | |
| | VH CDR2 | IDVGGSL | 12 |
| | VH CDR3 | ARGGLTYGFDL | 13 |
| | VL CDR1 | EDISSY | 14 |
| | VL CDR2 | GAS | 15 |
| | VL CDR3 | HYYATISGLGVA | 16 |
| | VH | QSLEESGGRLVTPGTPLTLTCTVSGFSLNDYWMSWV | 17 |
| | | RQAPGKGLEWIGYIDVGGSLYYASWAKGRFTISRTST | |
| | | TVDLKMTSLTTEDTATYFCARGGLTYGFDLWGPGTLV | |
| | | TVSS | |
| | VL | DIVMTQTPASVSEPVGGTVTINCQASEDISSYLAWYQ | 18 |
| | | QKPGQRPKRLIYGASDLASGVPSRFSASGSGTEYALTI | |
| | | SDLESADAATYYCHYYATISGLGVAFGGGTEVVVK | |
| | VH | EVOLVESGGGLVQPGRSLRLSCTASGFSLNDYWMSW | 19 |
| | humanized | VRQAPGKGLEWVGYIDVGGSLYYAASVKGRFTISRDD | |
| | | SKSIAYLQMNSLKTEDTAVYYCARGGLTYGFDLWGQG | |
| | | TLVTVSS | |
| | VL | DIVMTQSPSSLSASVGDRVTITCQASEDISSYLAWYQQ | 20 |
| | humanized | KPGKAPKRLIYGASDLASGVPSRFSASGSGTDYTFTIS | |
| | | SLQPEDIATYYCHYYATISGLGVAFGGGTKVEIK | |
| IgG1-Fc | | ASTKGPSVFPLAPSSKSTSGGTAALGCLVKDYFPEPV | 21 |
| | | TVSWNSGALTSGVHTFPAVLQSSGLYSLSSVVTVPSS | |
| | | SLGTQTYICNVNHKPSNTKVDKRVEPKSCDKTHTCPP | |
| | | CPAPELLGGPSVFLFPPKPKDTLMISRTPEVTCVVVDV | |
| | | SHEDPEVKFNWYVDGVEVHNAKTKPREEQYNSTYRV | |
| | | VSVLTVLHQDWLNGKEYKCKVSNKALPAPIEKTISKAK | |
| | | GQPREPQVYTLPPSREEMTKNQVSLTCLVKGFYPSDI | |
| | | AVEWESNGQPENNYKTTPPVLDSDGSFFLYSKLTVDK | |
| | | SRWQQGNVFSCSVMHEALHNHYTQKSLSLSPGK | |
| IgG1-Fc_F405L | | ASTKGPSVFPLAPSSKSTSGGTAALGCLVKDYFPEPV | 22 |
| | | TVSWNSGALTSGVHTFPAVLQSSGLYSLSSVVTVPSS | |
| | | SLGTQTYICNVNHKPSNTKVDKRVEPKSCDKTHTCPP | |
| | | CPAPELLGGPSVFLFPPKPKDTLMISRTPEVTCVVVDV | |
| | | SHEDPEVKFNWYVDGVEVHNAKTKPREEQYNSTYRV | |
| | | VSVLTVLHQDWLNGKEYKCKVSNKALPAPIEKTISKAK | |
| | | GQPREPQVYTLPPSREEMTKNQVSLTCLVKGFYPSDI | |
| | | AVEWESNGQPENNYKTTPPVLDSDGSFLLYSKLTVDK | |
| | | SRWQQGNVFSCSVMHEALHNHYTQKSLSLSPGK | |
| IgG1-Fc_K409R | | ASTKGPSVFPLAPSSKSTSGGTAALGCLVKDYFPEPV | 23 |
| | | TVSWNSGALTSGVHTFPAVLQSSGLYSLSSVVTVPSS | |
| | | SLGTQTYICNVNHKPSNTKVDKRVEPKSCDKTHTCPP | |
| | | CPAPELLGGPSVFLFPPKPKDTLMISRTPEVTCVVVDV | |
| | | SHEDPEVKFNWYVDGVEVHNAKTKPREEQYNSTYRV | |
| | | VSVLTVLHQDWLNGKEYKCKVSNKALPAPIEKTISKAK | |
| | | GQPREPQVYTLPPSREEMTKNQVSLTCLVKGFYPSDI | |
| | | AVEWESNGQPENNYKTTPPVLDSDGSFFLYSRLTVDK | |
| | | SRWQQGNVFSCSVMHEALHNHYTQKSLSLSPGK | |
| IgG1-Fc_FEA | | ASTKGPSVFPLAPSSKSTSGGTAALGCLVKDYFPEPV | 24 |
| | | TVSWNSGALTSGVHTFPAVLQSSGLYSLSSVVTVPSS | |
| | | SLGTQTYICNVNHKPSNTKVDKRVEPKSCDKTHTCPP | |
| | | CPAPEFEGGPSVFLFPPKPKDTLMISRTPEVTCVVVAV | |
| | | SHEDPEVKFNWYVDGVEVHNAKTKPREEQYNSTYRV | |
| | | VSVLTVLHQDWLNGKEYKCKVSNKALPAPIEKTISKAK | |
| | | GQPREPQVYTLPPSREEMTKNQVSLTCLVKGFYPSDI | |
| | | AVEWESNGQPENNYKTTPPVLDSDGSFFLYSKLTVDK | |
| | | SRWQQGNVFSCSVMHEALHNHYTQKSLSLSPGK | |
| IgG1-FEAL-Fc | | ASTKGPSVFPLAPSSKSTSGGTAALGCLVKDYFPEPV | 25 |
| | | TVSWNSGALTSGVHTFPAVLQSSGLYSLSSVVTVPSS | |
| | | SLGTQTYICNVNHKPSNTKVDKRVEPKSCDKTHTCPP | |
| | | CPAPEFEGGPSVFLFPPKPKDTLMISRTPEVTCVVVAV | |
| | | SHEDPEVKFNWYVDGVEVHNAKTKPREEQYNSTYRV | |
| | | VSVLTVLHQDWLNGKEYKCKVSNKALPAPIEKTISKAK | |
| | | GQPREPQVYTLPPSREEMTKNQVSLTCLVKGFYPSDI | |
| | | AVEWESNGQPENNYKTTPPVLDSDGSFLLYSKLTVDK | |
| | | SRWQQGNVFSCSVMHEALHNHYTQKSLSLSPGK | |
| IgG1-FEAR-Fc | | ASTKGPSVFPLAPSSKSTSGGTAALGCLVKDYFPEPV | 26 |
| | | TVSWNSGALTSGVHTFPAVLQSSGLYSLSSVVTVPSS | |
| | | SLGTQTYICNVNHKPSNTKVDKRVEPKSCDKTHTCPP | |
| | | CPAPEFEGGPSVFLFPPKPKDTLMISRTPEVTCVVVAV | |
| | | SHEDPEVKFNWYVDGVEVHNAKTKPREEQYNSTYRV | |
| | | VSVLTVLHQDWLNGKEYKCKVSNKALPAPIEKTISKAK | |
| | | GQPREPQVYTLPPSREEMTKNQVSLTCLVKGFYPSDI | |
| | | AVEWESNGQPENNYKTTPPVLDSDGSFFLYSRLTVDK | |
| | | SRWQQGNVFSCSVMHEALHNHYTQKSLSLSPGK | |
| Kappa-C | | RTVAAPSVFIFPPSDEQLKSGTASVVCLLNNFYPREAK | 27 |
| | | VQWKVDNALQSGNSQESVTEQDSKDSTYSLSSTLTLS | |
| | | KADYEKHKVYACEVTHQGLSSPVTKSFNRGEC | |
| Lambda-C | | GQPKAAPSVTLFPPSSEELQANKATLVCLISDFYPGAV | 28 |
| | | TVAWKADSSPVKAGVETTTPSKQSNNKYAASSYLSLT | |
| | | PEQWKSHRSYSCQVTHEGSTVEKTVAPTECS | |
| IgG1-Fc without C- | | ASTKGPSVFPLAPSSKSTSGGTAALGCLVKDYFPEPV | 29 |
| terminal Lys | | TVSWNSGALTSGVHTFPAVLQSSGLYSLSSVVTVPSS | |
| | | SLGTQTYICNVNHKPSNTKVDKRVEPKSCDKTHTCPP | |
| | | CPAPELLGGPSVFLFPPKPKDTLMISRTPEVTCVVVDV | |
| | | SHEDPEVKFNWYVDGVEVHNAKTKPREEQYNSTYRV | |
| | | VSVLTVLHQDWLNGKEYKCKVSNKALPAPIEKTISKAK | |
| | | GQPREPQVYTLPPSREEMTKNQVSLTCLVKGFYPSDI | |
| | | AVEWESNGQPENNYKTTPPVLDSDGSFFLYSKLTVDK | |
| | | SRWQQGNVFSCSVMHEALHNHYTQKSLSLSPG | |
| IgG1-Fc_F405L | | ASTKGPSVFPLAPSSKSTSGGTAALGCLVKDYFPEPV | 30 |
| without C-terminal | | TVSWNSGALTSGVHTFPAVLQSSGLYSLSSVVTVPSS | |
| Lys | | SLGTQTYICNVNHKPSNTKVDKRVEPKSCDKTHTCPP | |
| | | CPAPELLGGPSVFLFPPKPKDTLMISRTPEVTCVVVDV | |
| | | SHEDPEVKFNWYVDGVEVHNAKTKPREEQYNSTYRV | |
| | | VSVLTVLHQDWLNGKEYKCKVSNKALPAPIEKTISKAK | |
| | | GQPREPQVYTLPPSREEMTKNQVSLTCLVKGFYPSDI | |
| | | AVEWESNGQPENNYKTTPPVLDSDGSFLLYSKLTVDK | |
| | | SRWQQGNVFSCSVMHEALHNHYTQKSLSLSPG | |
| IgG1-Fc_K409R | | ASTKGPSVFPLAPSSKSTSGGTAALGCLVKDYFPEPV | 31 |
| without C-terminal | | TVSWNSGALTSGVHTFPAVLQSSGLYSLSSVVTVPSS | |
| Lys | | SLGTQTYICNVNHKPSNTKVDKRVEPKSCDKTHTCPP | |
| | | CPAPELLGGPSVFLFPPKPKDTLMISRTPEVTCVVVDV | |
| | | SHEDPEVKFNWYVDGVEVHNAKTKPREEQYNSTYRV | |
| | | VSVLTVLHQDWLNGKEYKCKVSNKALPAPIEKTISKAK | |
| | | GQPREPQVYTLPPSREEMTKNQVSLTCLVKGFYPSDI | |
| | | AVEWESNGQPENNYKTTPPVLDSDGSFFLYSRLTVDK | |
| | | SRWQQGNVFSCSVMHEALHNHYTQKSLSLSPG | |
| IgG1-Fc_FEA | | ASTKGPSVFPLAPSSKSTSGGTAALGCLVKDYFPEPV | 32 |
| without C-terminal | | TVSWNSGALTSGVHTFPAVLQSSGLYSLSSVVTVPSS | |
| Lys | | SLGTQTYICNVNHKPSNTKVDKRVEPKSCDKTHTCPP | |
| | | CPAPEFEGGPSVFLFPPKPKDTLMISRTPEVTCVVVAV | |
| | | SHEDPEVKFNWYVDGVEVHNAKTKPREEQYNSTYRV | |
| | | VSVLTVLHQDWLNGKEYKCKVSNKALPAPIEKTISKAK | |
| | | GQPREPQVYTLPPSREEMTKNQVSLTCLVKGFYPSDI | |
| | | AVEWESNGQPENNYKTTPPVLDSDGSFFLYSKLTVDK | |
| | | SRWQQGNVFSCSVMHEALHNHYTQKSLSLSPG | |
| IgG1-FEAL-Fc | | ASTKGPSVFPLAPSSKSTSGGTAALGCLVKDYFPEPV | 33 |
| without C-terminal | | TVSWNSGALTSGVHTFPAVLQSSGLYSLSSVVTVPSS | |
| Lys | | SLGTQTYICNVNHKPSNTKVDKRVEPKSCDKTHTCPP | |
| | | CPAPEFEGGPSVFLFPPKPKDTLMISRTPEVTCVVVAV | |
| | | SHEDPEVKFNWYVDGVEVHNAKTKPREEQYNSTYRV | |
| | | VSVLTVLHQDWLNGKEYKCKVSNKALPAPIEKTISKAK | |
| | | GQPREPQVYTLPPSREEMTKNQVSLTCLVKGFYPSDI | |
| | | AVEWESNGQPENNYKTTPPVLDSDGSFLLYSKLTVDK | |
| | | SRWQQGNVFSCSVMHEALHNHYTQKSLSLSPG | |
| IgG1-FEAR-Fc | | ASTKGPSVFPLAPSSKSTSGGTAALGCLVKDYFPEPV | 34 |
| without C-terminal | | TVSWNSGALTSGVHTFPAVLQSSGLYSLSSVVTVPSS | |
| Lys | | SLGTQTYICNVNHKPSNTKVDKRVEPKSCDKTHTCPP | |
| | | CPAPEFEGGPSVFLFPPKPKDTLMISRTPEVTCVVVAV | |
| | | SHEDPEVKFNWYVDGVEVHNAKTKPREEQYNSTYRV | |
| | | VSVLTVLHQDWLNGKEYKCKVSNKALPAPIEKTISKAK | |
| | | GQPREPQVYTLPPSREEMTKNQVSLTCLVKGFYPSDI | |
| | | AVEWESNGQPENNYKTTPPVLDSDGSFFLYSRLTVDK | |
| | | SRWQQGNVFSCSVMHEALHNHYTQKSLSLSPG | |
| Human CD40 | | MVRLPLQCVLWGCLLTAVHPEPPTACREKQYLINSQC | 35 |
| precursor | | CSLCQPGQKLVSDCTEFTETECLPCGESEFLDTWNRE | |
| | | THCHQHKYCDPNLGLRVQQKGTSETDTICTCEEGWH | |
| | | CTSEACESCVLHRSCSPGFGVKQIATGVSDTICEPCP | |
| | | VGFFSNVSSAFEKCHPWTSCETKDLVVQQAGTNKTD | |
| | | VVCGPQDRLRALVVIPIIFGILFAILLVLVFIKKVAKKPTN | |
| | | KAPHPKQEPQEINFPDDLPGSNTAAPVQETLHGCQPV | |
| | | TQEDGKESRISVQERQ | |
| Human CD40 | | EPPTACREKQYLINSQCCSLCQPGQKLVSDCTEFTET | 36 |
| mature protein | | ECLPCGESEFLDTWNRETHCHQHKYCDPNLGLRVQQ | |
| | | KGTSETDTICTCEEGWHCTSEACESCVLHRSCSPGFG | |
| | | VKQIATGVSDTICEPCPVGFFSNVSSAFEKCHPWTSC | |
| | | ETKDLVVQQAGTNKTDVVCGPQDRLRALVVIPIIFGILF | |
| | | AILLVLVFIKKVAKKPTNKAPHPKQEPQEINFPDDLPGS | |
| | | NTAAPVQETLHGCQPVTQEDGKESRISVQERQ | |
| Human CD137 | | MGNSCYNIVATLLLVLNFERTRSLQDPCSNCPAGTFC | 37 |
| precursor | | DNNRNQICSPCPPNSFSSAGGQRTCDICRQCKGVFR | |
| | | TRKECSSTSNAECDCTPGFHCLGAGCSMCEQDCKQ | |
| | | GQELTKKGCKDCCFGTFNDQKRGICRPWTNCSLDGK | |
| | | SVLVNGTKERDVVCGPSPADLSPGASSVTPPAPAREP | |
| | | GHSPQIISFFLALTSTALLFLLFFLTLRFSVVKRGRKKLL | |
| | | YIFKQPFMRPVQTTQEEDGCSCRFPEEEEGGCEL | |
| Human CD137 | | LQDPCSNCPAGTFCDNNRNQICSPCPPNSFSSAGGQ | 38 |
| mature protein | | RTCDICRQCKGVFRTRKECSSTSNAECDCTPGFHCLG | |
| | | AGCSMCEQDCKQGQELTKKGCKDCCFGTFNDQKRGI | |
| | | CRPWTNCSLDGKSVLVNGTKERDVVCGPSPADLSPG | |
| | | ASSVTPPAPAREPGHSPQIISFFLALTSTALLFLLFFLTL | |
| | | RFSWVKRGRKKLLYIFKQPFMRPVQTTQEEDGCSCRF | |
| | | PEEEEGGCEL | |

### DETAILED DESCRIPTION OF THE INVENTION

Although the present disclosure is further described in more detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

In the following, the elements of the present disclosure will be described in more detail. These elements are listed with specific embodiments, however, it should be understood that they may be combined in any manner and in any number to create additional embodiments. The variously described examples and preferred embodiments should not be construed to limit the present disclosure to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise. For example, if in a preferred embodiment of the binding agent used herein the first heavy chain comprises or consists essentially of or consists of an amino acid sequence set forth in SEQ ID NO: 26 or 34 [IgG1-Fc_FEAR] and in another preferred embodiment of the binding agent used herein the second heavy chain comprises or consists essentially of or consists of an amino acid sequence set forth in SEQ ID NO: 25 or 33 [IgG1-Fc_FEAL], then in a further preferred embodiment of the binding agent used herein the first heavy chain comprises or consists essentially of or consists of an amino acid sequence set forth in SEQ ID NO: 26 or 34 [IgG1-Fc_FEAR] and the second heavy chain comprises or consists essentially of or consists of an amino acid sequence set forth in SEQ ID NO: 25 or 33 [IgG1-Fc_FEAL].

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present disclosure will employ, unless otherwise indicated, conventional chemistry, biochemistry, cell biology, immunology, and recombinant DNA techniques which are explained in the literature in the field (cf, e.g., Organikum, Deutscher Verlag der Wissenschaften, Berlin 1990; Streitwieser/Heathcook, “Organische Chemie”, VCH, 1990; Beyer/Walter, “Lehrbuch der Organischen Chemie”, S. Hirzel Verlag Stuttgart, 1988; Carey/Sundberg, “Organische Chemie”, VCH, 1995; March, “Advanced Organic Chemistry”, John Wiley & Sons, 1985; Römpp Chemie Lexikon, Falbe/Regitz (Hrsg.), Georg Thieme Verlag Stuttgart, New York, 1989; Molecular Cloning: A Laboratory Manual, 2nd Edition, J. Sambrook et al. eds., Cold Spring Harbor Laboratory Press, Cold Spring Harbor 1989.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The term “consisting essentially of” means excluding other members, integers or steps of any essential significance. The term “comprising” encompasses the term “consisting essentially of” which, in turn, encompasses the term “consisting of”. Thus, at each occurrence in the present application, the term “comprising” may be replaced with the term “consisting essentially of” or “consisting of”. Likewise, at each occurrence in the present application, the term “consisting essentially of” may be replaced with the term “consisting of”.

The terms “a”, “an” and “the” and similar references used in the context of describing the present disclosure (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by the context. The use of any and all examples, or exemplary language (e.g., “such as”), provided herein is intended merely to better illustrate the present disclosure and does not pose a limitation on the scope of the present disclosure otherwise claimed. No language in the specification should be construed as indicating any non-claimed element essential to the practice of the present disclosure.

Where used herein, “and/or” is to be taken as specific disclosure of each of the two specified features or components with or without the other. For example, “X and/or Y” is to be taken as specific disclosure of each of (i) X, (ii) Y, and (iii) X and Y, just as if each is set out individually herein.

In the context of the present disclosure, the term “about” denotes an interval of accuracy that the person of ordinary skill will understand to still ensure the technical effect of the feature in question. The term typically indicates deviation from the indicated numerical value by ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, and for example ±0.01%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

Recitation of ranges of values herein is merely intended to serve as a shorthand method of referring individually to each separate value falling within the range. Unless otherwise indicated herein, each individual value is incorporated into the specification as if it were individually recited herein. Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### Definitions

In the following, definitions will be provided which apply to all aspects of the present disclosure. The following terms have the following meanings unless otherwise indicated. Any undefined terms have their art recognized meanings.

The term “binding agent” in the context of the present disclosure refers to any agent capable of binding to desired antigens. In certain embodiments of the present disclosure, the binding agent is an antibody, antibody fragment, or construct thereof. The binding agent may also comprise synthetic, modified or non-naturally occurring moieties, in particular non-peptide moieties. Such moieties may, for example, link desired antigen-binding functionalities or regions such as antibodies or antibody fragments. In one embodiment, the binding agent is a synthetic construct comprising antigen-binding CDRs or variable regions.

The term “immunoglobulin” relates to proteins of the immunoglobulin superfamily, preferably to antigen receptors such as antibodies or the B cell receptor (BCR). The immunoglobulins are characterized by a structural domain, i.e., the immunoglobulin domain, having a characteristic immunoglobulin (Ig) fold. The term encompasses membrane bound immunoglobulins as well as soluble immunoglobulins. Membrane bound immunoglobulins are also termed surface immunoglobulins or membrane immunoglobulins, which are generally part of the BCR. Soluble immunoglobulins are generally termed antibodies.

The structure of immunoglobulins has been well characterized. See, e.g., Fundamental Immunology Ch. 7 (Paul, W., ed., 2^{nd} ed. Raven Press, N.Y. (1989)). Briefly, immunoglobulins generally comprise several chains, typically two identical heavy chains and two identical light chains which are linked via disulfide bonds. These chains are primarily composed of immunoglobulin domains or regions, such as the V_{L} or VL (variable light chain) domain/region, C_{L} or CL (constant light chain) domain/region, V_{H} or VH (variable heavy chain) domain/region, and the C_{H} or CH (constant heavy chain) domains/regions C_{H}1 (CH1), C_{H}2 (CH2), C_{H}3 (CH3), and C_{H}4 (CH4). The heavy chain constant region typically is comprised of three domains, CH1, CH2, and CH3. The hinge region is the region between the CH1 and CH2 domains of the heavy chain and is highly flexible. Disulfide bonds in the hinge region are part of the interactions between two heavy chains in an IgG molecule. Each light chain typically is comprised of a VL and a CL. The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). Unless otherwise stated or contradicted by context, CDR sequences herein are identified according to IMGT rules using DomainGapAlign (Lefranc M P., Nucleic Acids Research 1999; 27:209-212 and Ehrenmann F., Kaas Q. and Lefranc M.-P. Nucleic Acids Res., 38, D301-307 (2010); see also internet http address www.imgt.org/i. Unless otherwise stated or contradicted by context, reference to amino acid positions in the constant regions in the present disclosure is according to the EU-numbering (Edelman et al., Proc Natl Acad Sci USA. 1969 May; 63(1):78-85; Kabat et al., Sequences of Proteins of Immunological Interest, Fifth Edition. 1991 NIH Publication No. 91-3242).

There are five types of mammalian immunoglobulin heavy chains, i.e., α, δ, ε, γ, and μ which account for the different classes of antibodies, i.e., IgA, IgD, IgE, IgG, and IgM. As opposed to the heavy chains of soluble immunoglobulins, the heavy chains of membrane or surface immunoglobulins comprise a transmembrane domain and a short cytoplasmic domain at their carboxy-terminus. In mammals there are two types of light chains, i.e., lambda and kappa. The immunoglobulin chains comprise a variable region and a constant region. The constant region is essentially conserved within the different isotypes of the immunoglobulins, wherein the variable part is highly divers and accounts for antigen recognition. The term “amino acid” and “amino acid residue” may herein be used interchangeably, and are not to be understood limiting. Amino acids are organic compounds containing amine (—NH₂) and carboxyl (—COOH) functional groups, along with a side chain (R group) specific to each amino acid. In the context of the present disclosure, amino acids may be classified based on structure and chemical characteristics. Thus, classes of amino acids may be reflected in one or both of the following tables:

TABLE 2
Main classification based on structure and general
chemical characterization of R group

| Class | Amino acid |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 3
Alternative Physical and Functional Classifications
of Amino Acid Residues

| Class | Amino acid |
|---|---|
| Hydroxyl group containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, |
| | W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, R, S, |
| | P, and T |
| Flexible residues | Q, T, K, S, G, P, D, E, and R |

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all mutants, splice variants, posttranslationally modified variants, conformations, isoforms, allelic variants, species variants, and species homologs, in particular those which are naturally occurring. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible.

Amino acid addition variants comprise amino- and/or carboxy-terminal fusions of one or more amino acids, such as 1, 2, 3, 5, 10, 20, 30, 50, or more amino acids.

Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants.

Amino acid substitution variants are characterized by at least one residue in the sequence being removed and another residue being inserted in its place. Substitution of one amino acid for another may be classified as a conservative or non-conservative substitution. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains. In the context of the present disclosure, a “conservative substitution” is a substitution of one amino acid with another amino acid having similar structural and/or chemical characteristics, such substitution of one amino acid residue for another amino acid residue of the same class as defined in any of the two tables above: for example, leucine may be substituted with isoleucine as they are both aliphatic, branched hydrophobes. Similarly, aspartic acid may be substituted with glutamic acid since they are both small, negatively charged residues. Naturally occurring amino acids may also be generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In one embodiment, conservative amino acid substitutions include substitutions within the following groups:
glycine, alanine;
valine, isoleucine, leucine;
aspartic acid, glutamic acid;
asparagine, glutamine;
serine, threonine;
lysine, arginine; and
phenylalanine, tyrosine.

The term “amino acid corresponding to position . . . ” as used herein refers to an amino acid position number in a human IgG1 heavy chain. Corresponding amino acid positions in other immunoglobulins may be found by alignment with human IgG1. Thus, an amino acid or segment in one sequence that “corresponds to” an amino acid or segment in another sequence is one that aligns with the other amino acid or segment using a standard sequence alignment program such as ALIGN, ClustalW or similar, typically at default settings and has at least 50%, at least 80%, at least 90%, or at least 95% identity to a human IgG1 heavy chain. It is considered well-known in the art how to align a sequence or segment in a sequence and thereby determine the corresponding position in a sequence to an amino acid position according to the present disclosure.

The term “antibody” (Ab) in the context of the present disclosure refers to an immunoglobulin molecule, a fragment of an immunoglobulin molecule, or a derivative of either thereof, which has the ability to specifically bind to an antigen (in particular an epitope on an antigen) under typical physiological conditions, preferably with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). In particular, the term “antibody” refers to a glycoprotein comprising at least two heavy (H) chains and two light (L) chains inter-connected by disulfide bonds. The term “antibody” includes monoclonal antibodies, recombinant antibodies, human antibodies, humanized antibodies, chimeric antibodies and combinations of any of the foregoing. Each heavy chain is comprised of a heavy chain variable region (VH) and a heavy chain constant region (CH). Each light chain is comprised of a light chain variable region (VL) and a light chain constant region (CL). The variable regions and constant regions are also referred to herein as variable domains and constant domains, respectively. The VH and VL regions can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. The CDRs of a VH are termed HCDR1, HCDR2 and HCDR3, the CDRs of a VL are termed LCDR1, LCDR2 and LCDR3. The variable regions of the heavy and light chains contain a binding domain that interacts with an antigen. The constant regions of an antibody comprise the heavy chain constant region (CH) and the light chain constant region (CL), wherein CH can be further subdivided into constant domain CH1, a hinge region, and constant domains CH2 and CH3 (arranged from amino-terminus to carboxy-terminus in the following order: CH1, CH2, CH3). The constant regions of the antibodies may mediate the binding of the immunoglobulin to host tissues or factors, including various cells of the immune system (e.g., effector cells) and components of the complement system such as C1q. Antibodies can be intact immunoglobulins derived from natural sources or from recombinant sources and can be immunoactive portions of intact immunoglobulins. Antibodies are typically tetramers of immunoglobulin molecules. Antibodies may exist in a variety of forms including, for example, polyclonal antibodies, monoclonal antibodies, Fv, Fab and F(ab)₂, as well as single chain antibodies and humanized antibodies.

The variable regions of the heavy and light chains of the immunoglobulin molecule contain a binding domain that interacts with an antigen. The term “antigen-binding region”, where used herein, refers to the region which interacts with the antigen and comprises both a VH region and a VL region. An antibody as used herein comprises not only monospecific antibodies, but also multispecific antibodies which comprise multiple, such as two or more, e.g., three or more, different antigen-binding regions.

As indicated above, the term antibody herein, unless otherwise stated or clearly contradicted by context, includes fragments of an antibody that are antigen-binding fragments, i.e., retain the ability to specifically bind to the antigen. It has been shown that the antigen-binding function of an antibody may be performed by fragments of a full-length antibody. Examples of antigen-binding fragments encompassed within the term “antibody” include (i) a Fab′ or Fab fragment, a monovalent fragment consisting of the VL, VH, CL and CH1 domains, or a monovalent antibody as described in WO 2007/059782 (Genmab); (ii) F(ab′)₂ fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody; (v) a dAb fragment (Ward et al., Nature 341, 544-546 (1989)), which consists essentially of a VH domain and also called domain antibodies (Holt et al; Trends Biotechnol. 2003 November; 21(11):484-90); (vi) camelid or Nanobody molecules (Revets et al; Expert Opin Biol Ther. 2005 January; 5(1):111-24); and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise noted or clearly indicated by context. Although such fragments are generally included within the meaning of antibody, they collectively and each independently are unique features of the present disclosure, exhibiting different biological properties and utility. These and other useful antibody fragments in the context of the present disclosure, as well as bispecific formats of such fragments, are discussed further herein. It also should be understood that the term antibody, unless specified otherwise, also includes polyclonal antibodies, monoclonal antibodies (mAbs), antibody-like polypeptides, such as chimeric antibodies and humanized antibodies, and antibody fragments retaining the ability to specifically bind to the antigen (antigen-binding fragments) provided by any known technique, such as enzymatic cleavage, peptide synthesis, and recombinant techniques.

An antibody as generated can possess any isotype. As used herein, the term “isotype” refers to the immunoglobulin class (for instance IgG1, IgG2, IgG3, IgG4, IgD, IgA, IgE, or IgM) that is encoded by heavy chain constant region genes. When a particular isotype, e.g. IgG1, is mentioned herein, the term is not limited to a specific isotype sequence, e.g. a particular IgG1 sequence, but is used to indicate that the antibody is closer in sequence to that isotype, e.g. IgG1, than to other isotypes. Thus, e.g. an IgG1 antibody disclosed herein may be a sequence variant of a naturally-occurring IgG1 antibody, including variations in the constant regions.

The term “multispecific antibody” in the context of the present disclosure refers to an antibody having at least two different antigen-binding regions defined by different antibody sequences. In some embodiments, said different antigen-binding regions bind different epitopes on the same antigen. However, in preferred embodiments, said different antigen-binding regions bind different target antigens. In one embodiment, the multispecific antibody is a “bispecific antibody” or “bs”. A multispecific antibody, such as a bispeccific antibody, can be of any format, including any of the bispecific or multispecific antibody formats described herein below.

The term “full-length” when used in the context of an antibody indicates that the antibody is not a fragment, but contains all of the domains of the particular isotype normally found for that isotype in nature, e.g. the VH, CH1, CH2, CH3, hinge, VL and CL domains for an IgG1 antibody.

The term “human antibody”, as used herein, is intended to include antibodies having variable and framework regions derived from human germline immunoglobulin sequences and a human immunoglobulin constant domain. The human antibodies disclosed herein may include amino acid residues not encoded by human germline immunoglobulin sequences (e.g., mutations, insertions or deletions introduced by random or site-specific mutagenesis in vitro or by somatic mutation in vivo). However, the term “human antibody”, as used herein, is not intended to include antibodies in which CDR sequences derived from the germline of another non-human species, such as a mouse, have been grafted onto human framework sequences.

The term “chimeric antibody” as used herein, refers to an antibody wherein the variable region is derived from a non-human species (e.g. derived from rodents) and the constant region is derived from a different species, such as human. Chimeric antibodies may be generated by antibody engineering. “Antibody engineering” is a term used generically for different kinds of modifications of antibodies, and processes for antibody engineering are well-known for the skilled person. In particular, a chimeric antibody may be generated by using standard DNA techniques as described in Sambrook et al., 1989, Molecular Cloning: A laboratory Manual, New York: Cold Spring Harbor Laboratory Press, Ch. 15. Thus, the chimeric antibody may be a genetically or an enzymatically engineered recombinant antibody. It is within the knowledge of the skilled person to generate a chimeric antibody, and thus, generation of the chimeric antibody may be performed by other methods than those described herein. Chimeric monoclonal antibodies for therapeutic applications in humans are developed to reduce anticipated antibody immunogenicity of non-human antibodies, e.g. rodent antibodies. They may typically contain non-human (e.g. murine or rabbit) variable regions, which are specific for the antigen of interest, and human constant antibody heavy and light chain domains. The terms “variable region” or “variable domain” as used in the context of chimeric antibodies, refer to a region which comprises the CDRs and framework regions of both the heavy and light chains of an immunoglobulin, as described below.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO 92/22653 and EP 0 629 240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

As used herein, a protein which is “derived from” another protein, e.g., a parent protein, means that one or more amino acid sequences of the protein are identical or similar to one or more amino acid sequences in the other or parent protein. For example, in an antibody, binding arm, antigen-binding region, constant region, or the like which is derived from another or a parent antibody, binding arm, antigen-binding region, or constant region, one or more amino acid sequences are identical or similar to those of the other or parent antibody, binding arm, antigen-binding region, or constant region. Examples of such one or more amino acid sequences include, but are not limited to, those of the VH and VL CDRs and/or one or more or all of the framework regions, VH, VL, CL, hinge, or CH regions. For example, a humanized antibody can be described herein as “derived from” a non-human parent antibody, meaning that at least the VL and VH CDR sequences are identical or similar to the VH and VL CDR sequences of said non-human parent antibody. A chimeric antibody can be described herein as being “derived from” a non-human parent antibody, meaning that typically the VH and VL sequences may be identical or similar to those of the non-human parent antibody. Another example is a binding arm or an antigen-binding region which may be described herein as being “derived from” a particular parent antibody, meaning that said binding arm or antigen-binding region typically comprises identical or similar VH and/or VL CDRs, or VH and/or VL sequences to the binding arm or antigen-binding region of said parent antibody. As described elsewhere herein, however, amino acid modifications such as mutations can be made in the CDRs, constant regions or elsewhere in the antibody, binding arm, antigen-binding region or the like, to introduce desired characteristics. When used in the context of one or more sequences derived from a first or parent protein, a “similar” amino acid sequence preferably has a sequence identity of at least about 50%, such as at least about 60%, at least about 70%, at least about 80%, at least about 90%, at least about 95%, or at least about 97%, 98% or 99%.

Non-human antibodies can be generated in a number of different species, such as mouse, rabbit, chicken, guinea pig, llama and goat.

Monoclonal antibodies can be produced by a variety of techniques, including conventional monoclonal antibody methodology, e.g., the standard somatic cell hybridization technique of Kohler and Milstein, Nature 256: 495 (1975). Other techniques for producing monoclonal antibodies can be employed, e.g., viral or oncogenic transformation of B-lymphocytes or phage display techniques using libraries of antibody genes, and such methods are well known to a person skilled in the art.

Hybridoma production in such non-human species is a very well established procedure. Immunization protocols and techniques for isolation of splenocytes of immunized animals/non-human species for fusion are known in the art. Fusion partners (e.g., murine myeloma cells) and fusion procedures are also known. When used herein, unless contradicted by context, the term “Fab-arm” or “arm” refers to one heavy chain-light chain pair and is used interchangeably with “half molecules” herein.

The term “binding arm comprising an antigen-binding region” means an antibody molecule or fragment that comprises an antigen-binding region. Thus, a binding arm can comprise, e.g., the six VH and VL CDR sequences, the VH and VL sequences, a Fab or Fab′ fragment, or a Fab-arm.

When used herein, unless contradicted by context, the term “Fc region” refers to an antibody region consisting of the two Fc sequences of the heavy chains of an immunoglobulin, wherein said Fc sequences comprise at least a hinge region, a CH2 domain, and a CH3 domain. In one embodiment, the term “Fc region”, as used herein, refers to a region comprising, in the direction from the N- to C-terminal end of the antibody, at least a hinge region, a CH2 region and a CH3 region. An Fc region of the antibody may mediate the binding of the immunoglobulin to host tissues or factors, including various cells of the immune system (such as effector cells) and components of the complement system.

In the context of the present disclosure, the term “induce Fc-mediated effector function to a lesser extent” used in relation to an antibody, including a multispecific antibody, means that the antibody induces Fc-mediated effector functions, such function in particular being selected from the list of IgG Fc receptor (FcgammaR, FcγR) binding, C1q binding, ADCC or CDC, to a lesser extent compared to a human IgG1 antibody comprising (i) the same CDR sequences, in particular comprising the same first and second antigen-binding regions, as said antibody and (ii) two heavy chains comprising human IgG1 hinge, CH2 and CH3 regions.

Fc-mediated effector function may be measured by binding to FcγRs, binding to C1q, or induction of Fc-mediated cross-linking via FcγRs.

The term “hinge region” as used herein refers to the hinge region of an immunoglobulin heavy chain. Thus, for example, the hinge region of a human IgG1 antibody corresponds to amino acids 216-230 according to the EU numbering as set forth in Kabat (Kabat, E. A. et al., Sequences of proteins of immunological interest. 5th Edition—US Department of Health and Human Services, NIH publication No. 91-3242, pp 662,680,689 (1991). However, the hinge region may also be any of the other subtypes as described herein.

The term “CH1 region” or “CH1 domain” as used herein refers to the CH1 region of an immunoglobulin heavy chain. Thus, for example, the CH1 region of a human IgG1 antibody corresponds to amino acids 118-215 according to the EU numbering as set forth in Kabat (ibid). However, the CH1 region may also be any of the other subtypes as described herein.

The term “CH2 region” or “CH2 domain” as used herein refers to the CH2 region of an immunoglobulin heavy chain. Thus, for example, the CH2 region of a human IgG1 antibody corresponds to amino acids 231-340 according to the EU numbering as set forth in Kabat (ibid). However, the CH2 region may also be any of the other subtypes as described herein.

The term “CH3 region” or “CH3 domain” as used herein refers to the CH3 region of an immunoglobulin heavy chain. Thus, for example, the CH3 region of a human IgG1 antibody corresponds to amino acids 341-447 according to the EU numbering as set forth in Kabat (ibid). However, the CH3 region may also be any of the other subtypes as described herein.

The term “monovalent antibody” means in the context of the present disclosure that an antibody molecule is capable of binding a single molecule of the antigen, and thus is not capable of antigen cross-linking.

A “CD40 antibody” or “anti-CD40 antibody” is an antibody as described above, which binds specifically to the antigen CD40.

A “CD137 antibody” or “anti-CD137 antibody” is an antibody as described above, which binds specifically to the antigen CD137.

A “CD40×CD137 antibody” or “anti-CD40×CD137 antibody” is a bispecific antibody, which comprises two different antigen-binding regions, one of which binds specifically to the antigen CD40 and one of which binds specifically to the antigen CD137.

As used herein, the terms “binding” or “capable of binding” in the context of the binding of an antibody to a predetermined antigen or epitope typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less, when determined using Bio-Layer Interferometry (BLI) or, for instance, when determined using surface plasmon resonance (SPR) technology in a BIAcore 3000 instrument using the antigen as the ligand and the antibody as the analyte. The antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} for binding to a non-specific antigen (e.g., BSA, casein) other than the predetermined antigen or a closely-related antigen. The amount with which the affinity is higher is dependent on the K_{D} of the antibody, so that when the K_{D} of the antibody is very low (that is, the antibody is highly specific), then the degree to which the affinity for the antigen is lower than the affinity for a non-specific antigen may be at least 10,000-fold.

The term “k_{d}” (sec⁻¹), as used herein, refers to the dissociation rate constant of a particular antibody-antigen interaction. Said value is also referred to as the k_{off} value.

The term “K_{D}” (M), as used herein, refers to the dissociation equilibrium constant of a particular antibody-antigen interaction.

Two antibodies have the “same specificity” if they bind to the same antigen and to the same epitope. Whether an antibody to be tested recognizes the same epitope as a certain antigen-binding antibody, i.e., the antibodies bind to the same epitope, may be tested by different methods well known to a person skilled in the art.

The competition between the antibodies can be detected by a cross-blocking assay. For example, a competitive ELISA assay may be used as a cross-blocking assay. E.g., target antigen may be coated on the wells of a microtiter plate and antigen-binding antibody and candidate competing test antibody may be added. The amount of the antigen-binding antibody bound to the antigen in the well indirectly correlates with the binding ability of the candidate competing test antibody that competes therewith for binding to the same epitope. Specifically, the larger the affinity of the candidate competing test antibody is for the same epitope, the smaller the amount of the antigen-binding antibody bound to the antigen-coated well. The amount of the antigen-binding antibody bound to the well can be measured by labeling the antibody with detectable or measurable labeling substances.

An antibody competing for binding to an antigen with another antibody, e.g., an antibody comprising heavy and light chain variable regions as described herein, or an antibody having the specificity for an antigen of another antibody, e.g., an antibody comprising heavy and light chain variable regions as described herein, may be an antibody comprising variants of said heavy and/or light chain variable regions as described herein, e.g. modifications in the CDRs and/or a certain degree of identity as described herein.

An “isolated multispecific antibody” as used herein is intended to refer to a multispecific antibody which is substantially free of other antibodies having different antigenic specificities (for instance an isolated bispecific antibody that specifically binds to CD40 and CD137 is substantially free of monospecific antibodies that specifically bind to CD40 or CD137).

The term “monoclonal antibody” as used herein refers to a preparation of antibody molecules of single molecular composition. A monoclonal antibody composition displays a single binding specificity and affinity for a particular epitope.

When used herein the term “heterodimeric interaction between the first and second CH3 regions” refers to the interaction between the first CH3 region and the second CH3 region in a first-CH3/second-CH3 heterodimeric antibody.

When used herein the term “homodimeric interactions of the first and second CH3 regions” refers to the interaction between a first CH3 region and another first CH3 region in a first-CH3/first-CH3 homodimeric antibody and the interaction between a second CH3 region and another second CH3 region in a second-CH3/second-CH3 homodimeric antibody.

When used herein the term “homodimeric antibody” refers to an antibody comprising two first Fab-arms or half-molecules, wherein the amino acid sequence of said Fab-arms or half-molecules is the same.

When used herein the term “heterodimeric antibody” refers to an antibody comprising a first and a second Fab-arm or half-molecule, wherein the amino acid sequence of said first and second Fab-arms or half-molecules are different. In particular, the CH3 region, or the antigen-binding region, or the CH3 region and the antigen-binding region of said first and second Fab-arms/half-molecules are different.

The term “reducing conditions” or “reducing environment” refers to a condition or an environment in which a substrate, such as a cysteine residue in the hinge region of an antibody, is more likely to become reduced than oxidized.

The present disclosure also describes multispecific antibodies, such as bispecific antibodies, comprising functional variants of the VL regions, VH regions, or one or more CDRs of the bispecific antibodies of the examples. A functional variant of a VL, VH, or CDR used in the context of a bispecific antibody still allows each antigen-binding region of the bispecific antibody to retain at least a substantial proportion (at least about 50%, 60%, 70%, 80%, 90%, 95% or more) of the affinity and/or the specificity/selectivity of the parent bispecific antibody and in some cases such a bispecific antibody may be associated with greater affinity, selectivity and/or specificity than the parent bispecific antibody.

Such functional variants typically retain significant sequence identity to the parent bispecific antibody. The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444-453 (1970) algorithm. In the context of the present disclosure, the following notations are, unless otherwise indicated, used to describe a mutation: i) substitution of an amino acid in a given position is written as e.g. K409R which means a substitution of a lysine in position 409 of the protein with an arginine; and ii) for specific variants the specific three or one letter codes are used, including the codes Xaa and X to indicate any amino acid residue. Thus, the substitution of lysine with arginine in position 409 is designated as: K409R, and the substitution of lysine with any amino acid residue in position 409 is designated as K409X. In case of deletion of lysine in position 409 it is indicated by K409*.

Exemplary variants include those which differ from the VH and/or VL and/or CDRs of the parent sequences mainly by conservative substitutions; for example, 12, such as 11, 10, 9, 8, 7, 6, 5, 4, 3, 2 or 1 of the substitutions in the variant are conservative amino acid residue replacements.

In the context of the present disclosure, conservative substitutions may be defined by substitutions within the classes of amino acids as defined in tables 2 and 3.

The term “CD40” as used herein, refers to CD40, also referred to as tumor necrosis factor receptor superfamily member 5 (TNFRSF5), which is the receptor for the ligand TNFSF5/CD40L. CD40 is known to transduce TRAF6- and MAP3K8-mediated signals that activate ERK in macrophages and B cells, leading to induction of immunoglobulin secretion by the B cells. Other synonyms used for CD40 include, but are not limited to, B-cell surface antigen CD40, Bp50, CD40L receptor and CDw40. In one embodiment, CD40 is human CD40, having UniProt accession number P25942. The sequence of human CD40 is also shown in SEQ ID NO: 35. Amino acids 1-20 of SEQ ID NO: 35 correspond to the signal peptide of human CD40; while amino acids 21-193 of SEQ ID NO: 35 correspond to the extracellular domain of human CD40; and the remainder of the protein; i.e. from amino acids 194-215 and 216-277 of SEQ ID NO: 35 is transmembrane and cytoplasmic domain, respectively.

The term “CD137” as used herein, refers to CD137 (4-1BB), also referred to as tumor necrosis factor receptor superfamily member 9 (TNFRSF9), which is the receptor for the ligand TNFSF9/4-1BBL. CD137 (4-1BB) is believed to be involved in T-cell activation. Other synonyms for CD137 include, but are not limited to, 4-1BB ligand receptor, CDw137, T-cell antigen 4-1BB homolog and T-cell antigen ILA. In one embodiment, CD137 (4-1BB) is human CD137 (4-1BB), having UniProt accession number Q07011. The sequence of human CD137 is also shown in SEQ ID NO: 37. Amino acids 1-23 of SEQ ID NO: 37 correspond to the signal peptide of human CD137; while amino acids 24-186 of SEQ ID NO: 37 correspond to the extracellular domain of human CD137; and the remainder of the protein, i.e. from amino acids 187-213 and 214-255 of SEQ ID NO: 37 are transmembrane and cytoplasmic domain, respectively.

“Treatment cycle” is herein defined as the time period, within the effects of separate dosages of the binding agent adds on, or are essentially additive, due to the pharmacodynamics of the binding agent, or in other words the time period after which the administrated binding agent is essentially cleared from the subject's body. Multiple small doses in a small time window, e.g. within 2-24 few hours, such as 2-12 hours or on the same day, might be equal to a larger single dose.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications. In one embodiment, “treatment” refers to the administration of an effective amount of a therapeutically active binding agent, such as of a therapeutically active antibody, of the present disclosure with the purpose of easing, ameliorating, arresting or eradicating (curing) symptoms or disease states.

The resistance to, failure to respond to and/or relapse from treatment with a binding agent of the present disclosure may be determined according to the Response Evaluation Criteria in Solid Tumors; version 1.1 (RECIST Criteria v1.1). The RECIST Criteria are set forth in the table below (LD: longest dimension).

TABLE 4
Definition of Response (RECIST Criteria v1.1)

| | Category | Criteria |
|---|---|---|
| Based on target | Complete Response | Disappearance of all target lesions. Any pathological lymph |
| lesions | (CR) | nodes must have reduction in short axis to <10 mm. |
| | Partial Response | ≥30% decrease in the sum of the LD of target lesions, |
| | (PR) | taking as reference the baseline sum LD. |
| | Stable Disease | Neither sufficient shrinkage to qualify for PR nor sufficient |
| | (SD) | increase to qualify for PD, taking as reference the smallest |
| | | sum of LDs since the treatment started. |
| | Progressive Disease | ≥20% increase in the sum of the LDs of target lesions, |
| | (PD) | taking as reference the smallest sum of the LDs recorded |
| | | since the treatment started or the appearance of one or more |
| | | new lesions. |
| Based on non- | CR | Disappearance of all non-target lesions and normalization of |
| target lesions | | tumor marker level. All lymph nodes must be non- |
| | | pathological in size (<10 mm short axis). |
| | Category | Criteria |
| | SD | Persistence of one or more non-target lesion(s) or/and |
| | | maintenance of tumor marker level above the normal limits. |
| | PD | Appearance of one or more new lesions and/or unequivocal |
| | | progression of existing non-target lesions. |

The “best overall response” is the best response recorded from the start of the treatment until disease progression/recurrence (the smallest measurements recorded since the treatment started will be used as the reference for PD). Subjects with CR or PR are considered to have an objective response. Subjects with CR, PR or SD are considered to be in disease control. Subjects with NE are counted as non-responders. The best overall response is the best response recorded from the start of the treatment until disease progression/recurrence (the smallest measurements recorded since the treatment started will be used as the reference for PD).

“Duration of response (DOR)” only applies to subjects whose confirmed best overall response is CR or PR and is defined as the time from the first documentation of objective tumor response (CR or PR) to the date of first PD or death due to underlying cancer.

“Progression-free survival (PFS)” is defined as the number of days from Day 1 in Cycle 1 to the first documented progression or death due to any cause.

“Overall survival (OS)” is defined as the number of days from Day 1 in Cycle 1 to death due to any cause. If a subject is not known to have died, then OS will be censored at the latest date the subject was known to be alive (on or before the cut-off date).

In the context of the present disclosure, the term “treatment regimen” refers to a structured treatment plan designed to improve and maintain health.

The term “effective amount” or “therapeutically effective amount” refers to an amount effective, at dosages and for periods of time necessary, to achieve a desired therapeutic result. A therapeutically effective amount of a binding agent (such as a multispecific, e.g., bispecific antibody) may vary according to factors such as the disease to be treated, the disease state, the severity of disease, the individual parameters of the patient (including age, sex, physiological condition, diet, and weight of the patient), the duration of treatment, the type of an accompanying therapy (if present), the specific route of administration, the ability of the binding agent (such as the multispecific, e.g., bispecific, antibody) to elicit a desired response in the patient, and similar factors. Accordingly, the doses administered of the agents described herein may depend on various of such parameters. A therapeutically effective amount is also one in which any toxic or detrimental effects of the binding agent (such as the multispecific, e.g., bispecific, antibody) or a fragment thereof, are outweighed by the therapeutically beneficial effects. In the case that a reaction in a patient is insufficient with an initial dose, higher doses (or effectively higher doses achieved by a different, more localized route of administration) may be used. In case that unwanted side effects occur in a patient with a dose, lower doses (or effectively lower doses achieved by a different, more localized route of administration) may be used.

As used herein, the term “cancer” includes a disease characterized by aberrantly regulated cellular growth, proliferation, differentiation, adhesion, and/or migration. By “cancer cell” is meant an abnormal cell that grows by a rapid, uncontrolled cellular proliferation and continues to grow after the stimuli that initiated the new growth cease.

The term “cancer” according to the present disclosure comprises leukemias, seminomas, melanomas, teratomas, lymphomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer and lung cancer and the metastases thereof. Examples thereof are lung carcinomas, mamma carcinomas, prostate carcinomas, colon carcinomas, renal cell carcinomas, cervical carcinomas, or metastases of the cancer types or tumors described above.

The term “cancer” according to the present disclosure also comprises cancer metastases. By “metastasis” is meant the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor, i.e. a secondary tumor or metastatic tumor, at the target site depends on angiogenesis. Tumor metastasis often occurs even after the removal of the primary tumor because tumor cells or components may remain and develop metastatic potential. In one embodiment, the term “metastasis” according to the present disclosure relates to “distant metastasis” which relates to a metastasis which is remote from the primary tumor and the regional lymph node system.

Terms such as “reduce” or “inhibit” as used herein means the ability to cause an overall decrease, for example, of about 5% or greater, about 10% or greater, about 15% or greater, about 20% or greater, about 25% or greater, about 30% or greater, about 40% or greater, about 50% or greater, or about 75% or greater, in the level. The term “inhibit” or similar phrases includes a complete or essentially complete inhibition, i.e. a reduction to zero or essentially to zero.

Terms such as “increase” or “enhance” in one embodiment relate to an increase or enhancement by at least about 10%, at least about 20%, at least about 30%, at least about 40%, at least about 50%, at least about 80%, or at least about 100%.

“Physiological pH” as used herein refers to a pH of about 7.5.

As used in the present disclosure, “% by weight” refers to weight percent, which is a unit of concentration measuring the amount of a substance in grams (g) expressed as a percent of the total weight of the total composition in grams (g).

The term “freezing” relates to the solidification of a liquid, usually with the removal of heat.

The term “lyophilizing” or “lyophilization” refers to the freeze-drying of a substance by freezing it and then reducing the surrounding pressure (e.g., below 15 Pa, such as below 10 Pa, below 5 Pa, or 1 Pa or less) to allow the frozen medium in the substance to sublimate directly from the solid phase to the gas phase. Thus, the terms “lyophilizing” and “freeze-drying” are used herein interchangeably.

The term “recombinant” in the context of the present disclosure means “made through genetic engineering”. In one embodiment, a “recombinant object” in the context of the present disclosure is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

According to the present disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” refers to large peptides, in particular peptides having at least about 151 amino acids, but the terms “peptide” and “protein” are used herein usually as synonyms.

A “therapeutic protein” has a positive or advantageous effect on a condition or disease state of a subject when provided to the subject in a therapeutically effective amount. In one embodiment, a therapeutic protein has curative or palliative properties and may be administered to ameliorate, relieve, alleviate, reverse, delay onset of or lessen the severity of one or more symptoms of a disease or disorder. A therapeutic protein may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Examples of therapeutically active proteins include, but are not limited to, antigens for vaccination and immunostimulants such as cytokines.

The term “portion” refers to a fraction. With respect to a particular structure such as an amino acid sequence or protein the term “portion” thereof may designate a continuous or a discontinuous fraction of said structure.

The terms “part” and “fragment” are used interchangeably herein and refer to a continuous element. For example, a part of a structure such as an amino acid sequence or protein refers to a continuous element of said structure. When used in context of a composition, the term “part” means a portion of the composition. For example, a part of a composition may any portion from 0.1% to 99.9% (such as 0.1%, 0.5%, 1%, 5%, 10%, 50%, 90%, or 99%) of said composition.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence.

According to the present disclosure, a part or fragment of a peptide or protein preferably has at least one functional property of the peptide or protein from which it has been derived. Such functional properties comprise a pharmacological activity, the interaction with other peptides or proteins, an enzymatic activity, the interaction with antibodies, and the selective binding of nucleic acids. E.g., a pharmacological active fragment of a peptide or protein has at least one of the pharmacological activities of the peptide or protein from which the fragment has been derived. A part or fragment of a peptide or protein preferably comprises a sequence of at least 6, in particular at least 8, at least 10, at least 12, at least 15, at least 20, at least 30 or at least 50, consecutive amino acids of the peptide or protein. A part or fragment of a peptide or protein preferably comprises a sequence of up to 8, in particular up to 10, up to 12, up to 15, up to 20, up to 30 or up to 55, consecutive amino acids of the peptide or protein.

By “variant” herein is meant an amino acid sequence that differs from a parent amino acid sequence by virtue of at least one amino acid modification. The parent amino acid sequence may be a naturally occurring or wild type (WT) amino acid sequence, or may be a modified version of a wild type amino acid sequence. Preferably, the variant amino acid sequence has at least one amino acid modification compared to the parent amino acid sequence, e.g., from 1 to about 20 amino acid modifications, and preferably from 1 to about 10 or from 1 to about 5 amino acid modifications compared to the parent.

By “wild type” or “WT” or “native” herein is meant an amino acid sequence that is found in nature, including allelic variations. A wild type amino acid sequence, peptide or protein has an amino acid sequence that has not been intentionally modified.

Preferably the degree of similarity, preferably identity between a given amino acid sequence and an amino acid sequence which is a variant of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, in some embodiments continuous amino acids. In some embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical” and “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq&LINK_LOC=align2seq). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, −2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of similarity or identity is given for a region which is at least about 50%, at least about 60%, at least about 70%, at least about 80%, at least about 90% or about 100% of the entire length of the reference sequence. For example, if the reference amino acid sequence consists of 200 amino acid residues, the degree of identity is given for at least about 100, at least about 120, at least about 140, at least about 160, at least about 180, or about 200 amino acid residues, in some embodiments continuous amino acid residues. In some embodiments, the degree of similarity or identity is given for the entire length of the reference sequence.

Homologous amino acid sequences exhibit according to the present disclosure at least 40%, in particular at least 50%, at least 60%, at least 70%, at least 80%, at least 90% and preferably at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or proteins having substitutions, additions, insertions or deletions, is described in detail in Sambrook et al. (1989), for example. Furthermore, the peptides and amino acid variants described herein may be readily prepared with the aid of known peptide synthesis techniques such as, for example, by solid phase synthesis and similar methods.

In one embodiment, a fragment or variant of an amino acid sequence (peptide or protein) is preferably a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to antigens or antigenic sequences, one particular function is one or more immunogenic activities displayed by the amino acid sequence from which the fragment or variant is derived. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In one embodiment, the modifications in the amino acid sequence of the parent molecule or sequence do not significantly affect or alter the characteristics of the molecule or sequence. In different embodiments, the function of the functional fragment or functional variant may be reduced but still significantly present, e.g., immunogenicity of the functional variant may be at least 50%, at least 60%, at least 70%, at least 80%, or at least 90% of the parent molecule or sequence. However, in other embodiments, immunogenicity of the functional fragment or functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. Preferably, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

“Isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell. In a preferred embodiment, the binding agent used in the present disclosure is in substantially purified form.

The term “genetic modification” or simply “modification” includes the transfection of cells with nucleic acid. The term “transfection” relates to the introduction of nucleic acids, in particular RNA, into a cell. For purposes of the present disclosure, the term “transfection” also includes the introduction of a nucleic acid into a cell or the uptake of a nucleic acid by such cell, wherein the cell may be present in a subject, e.g., a patient. Thus, according to the present disclosure, a cell for transfection of a nucleic acid described herein can be present in vitro or in vivo, e.g. the cell can form part of an organ, a tissue and/or an organism of a patient. According to the present disclosure, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection. Generally, nucleic acid encoding antigen is transiently transfected into cells. RNA can be transfected into cells to transiently express its coded protein.

According to the present disclosure, an analog of a peptide or protein is a modified form of said peptide or protein from which it has been derived and has at least one functional property of said peptide or protein. E.g., a pharmacological active analog of a peptide or protein has at least one of the pharmacological activities of the peptide or protein from which the analog has been derived. Such modifications include any chemical modification and comprise single or multiple substitutions, deletions and/or additions of any molecules associated with the protein or peptide, such as carbohydrates, lipids and/or proteins or peptides. In one embodiment, “analogs” of proteins or peptides include those modified forms resulting from glycosylation, acetylation, phosphorylation, amidation, palmitoylation, myristoylation, isoprenylation, lipidation, alkylation, derivatization, introduction of protective/blocking groups, proteolytic cleavage or binding to an antibody or to another cellular ligand. The term “analog” also extends to all functional chemical equivalents of said proteins and peptides.

“Activation” or “stimulation”, as used herein, refers to the state of an immune effector cell such as T cell that has been sufficiently stimulated to induce detectable cellular proliferation. Activation can also be associated with initiation of signaling pathways, induced cytokine production, and detectable effector functions. The term “activated immune effector cells” refers to, among other things, immune effector cells that are undergoing cell division.

The term “priming” refers to a process wherein an immune effector cell such as a T cell has its first contact with its specific antigen and causes differentiation into effector cells such as effector T cells.

The term “clonal expansion” or “expansion” refers to a process wherein a specific entity is multiplied. In the context of the present disclosure, the term is preferably used in the context of an immunological response in which immune effector cells are stimulated by an antigen, proliferate, and the specific immune effector cell recognizing said antigen is amplified. Preferably, clonal expansion leads to differentiation of the immune effector cells.

An “antigen” according to the present disclosure covers any substance that will elicit an immune response and/or any substance against which an immune response or an immune mechanism such as a cellular response is directed. This also includes situations wherein the antigen is processed into antigen peptides and an immune response or an immune mechanism is directed against one or more antigen peptides, in particular if presented in the context of MHC molecules. In particular, an “antigen” relates to any substance, preferably a peptide or protein, that reacts specifically with antibodies or T-lymphocytes (T-cells). According to the present disclosure, the term “antigen” comprises any molecule which comprises at least one epitope, such as a T cell epitope. Preferably, an antigen in the context of the present disclosure is a molecule which, optionally after processing, induces an immune reaction, which is preferably specific for the antigen (including cells expressing the antigen). In one embodiment, an antigen is a disease-associated antigen, such as a tumor antigen, a viral antigen, or a bacterial antigen, or an epitope derived from such antigen.

According to the present disclosure, any suitable antigen may be used, which is a candidate for an immune response, wherein the immune response may be both a humoral as well as a cellular immune response. In the context of some embodiments of the present disclosure, the antigen is preferably presented by a cell, preferably by an antigen presenting cell, in the context of MHC molecules, which results in an immune response against the antigen. An antigen is preferably a product which corresponds to or is derived from a naturally occurring antigen. Such naturally occurring antigens may include or may be derived from allergens, viruses, bacteria, fungi, parasites and other infectious agents and pathogens or an antigen may also be a tumor antigen. According to the present disclosure, an antigen may correspond to a naturally occurring product, for example, a viral protein, or a part thereof.

The term “disease-associated antigen” is used in its broadest sense to refer to any antigen associated with a disease. A disease-associated antigen is a molecule which contains epitopes that will stimulate a host's immune system to make a cellular antigen-specific immune response and/or a humoral antibody response against the disease. Disease-associated antigens include pathogen-associated antigens, i.e., antigens which are associated with infection by microbes, typically microbial antigens (such as bacterial or viral antigens), or antigens associated with cancer, typically tumors, such as tumor antigens.

In a preferred embodiment, the antigen is a tumor antigen, i.e., a part of a tumor cell, in particular those which primarily occur intracellularly or as surface antigens of tumor cells. In another embodiment, the antigen is a pathogen-associated antigen, i.e., an antigen derived from a pathogen, e.g., from a virus, bacterium, unicellular organism, or parasite, for example a viral antigen such as viral ribonucleoprotein or coat protein. In particular, the antigen should be presented by MHC molecules which results in modulation, in particular activation of cells of the immune system, preferably CD4⁺ and CD8⁺ lymphocytes, in particular via the modulation of the activity of a T-cell receptor.

The term “tumor antigen” refers to a constituent of cancer cells which may be derived from the cytoplasm, the cell surface or the cell nucleus. In particular, it refers to those antigens which are produced intracellularly or as surface antigens on tumor cells. For example, tumor antigens include the carcinoembryonal antigen, al-fetoprotein, isoferritin, and fetal sulphoglycoprotein, α2-H-ferroprotein and γ-fetoprotein, as well as various virus tumor antigens. According to the present disclosure, a tumor antigen preferably comprises any antigen which is characteristic for tumors or cancers as well as for tumor or cancer cells with respect to type and/or expression level.

The term “viral antigen” refers to any viral component having antigenic properties, i.e., being able to provoke an immune response in an individual. The viral antigen may be a viral ribonucleoprotein or an envelope protein.

The term “bacterial antigen” refers to any bacterial component having antigenic properties, i.e. being able to provoke an immune response in an individual. The bacterial antigen may be derived from the cell wall or cytoplasm membrane of the bacterium.

The term “epitope” refers to an antigenic determinant in a molecule such as an antigen, i.e., to a part in or fragment of the molecule that is recognized by the immune system, for example, that is recognized by antibodies T cells or B cells, in particular when presented in the context of MHC molecules. In one embodiment, “epitope” means a protein determinant capable of specific binding to an antibody. Epitopes usually consist of surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents. The epitope may comprise amino acid residues directly involved in the binding and other amino acid residues, which are not directly involved in the binding, such as amino acid residues which are effectively blocked or covered by the specifically antigen-binding peptide (in other words, the amino acid residue is within the footprint of the specifically antigen-binding peptide).

An epitope of a protein preferably comprises a continuous or discontinuous portion of said protein and is preferably between about 5 and about 100, preferably between about 5 and about 50, more preferably between about 8 and about 0, most preferably between about 10 and about 25 amino acids in length, for example, the epitope may be preferably 9, 10, 11, 12, 13, 14, 15, 16, 17, 18, 19, 20, 21, 22, 23, 24, or 25 amino acids in length. It is particularly preferred that the epitope in the context of the present disclosure is a T cell epitope.

Terms such as “epitope”, “fragment of an antigen”, “immunogenic peptide” and “antigen peptide” are used interchangeably herein and preferably relate to an incomplete representation of an antigen which is preferably capable of eliciting an immune response against the antigen or a cell expressing or comprising and preferably presenting the antigen. Preferably, the terms relate to an immunogenic portion of an antigen.

Preferably, it is a portion of an antigen that is recognized (i.e., specifically bound) by a T cell receptor, in particular if presented in the context of MHC molecules. Certain preferred immunogenic portions bind to an MHC class I or class II molecule. The term “epitope” refers to a part or fragment of a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, more preferably between about 8 and about 30, most preferably between about 8 and about 25 amino acids in length, for example, the epitope may be preferably 9, 10, 11, 12, 13, 14, 15, 16, 17, 18, 19, 20, 21, 22, 23, 24, or 25 amino acids in length. In one embodiment, an epitope is between about 10 and about 25 amino acids in length. The term “epitope” includes T cell epitopes.

The term “T cell epitope” refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

The peptide and protein antigen can be 2 to 100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide can be greater than 50 amino acids. In some embodiments, the peptide can be greater than 100 amino acids.

The peptide or protein antigen can be any peptide or protein that can induce or increase the ability of the immune system to develop antibodies and T cell responses to the peptide or protein.

In one embodiment, vaccine antigen, i.e., an antigen whose inoculation into a subject induces an immune response, is recognized by an immune effector cell. Preferably, the vaccine antigen if recognized by an immune effector cell is able to induce in the presence of appropriate co-stimulatory signals, stimulation, priming and/or expansion of the immune effector cell carrying an antigen receptor recognizing the vaccine antigen. In the context of the embodiments of the present disclosure, the vaccine antigen is preferably presented or present on the surface of a cell, preferably an antigen presenting cell. In one embodiment, an antigen is presented by a diseased cell (such as tumor cell or an infected cell). In one embodiment, an antigen receptor is a TCR which binds to an epitope of an antigen presented in the context of MHC. In one embodiment, binding of a TCR when expressed by T cells and/or present on T cells to an antigen presented by cells such as antigen presenting cells results in stimulation, priming and/or expansion of said T cells. In one embodiment, binding of a TCR when expressed by T cells and/or present on T cells to an antigen presented on diseased cells results in cytolysis and/or apoptosis of the diseased cells, wherein said T cells preferably release cytotoxic factors, e.g., perforins and granzymes.

In one embodiment, an antigen receptor is an antibody or B cell receptor which binds to an epitope in an antigen. In one embodiment, an antibody or B cell receptor binds to native epitopes of an antigen.

The term “expressed on the cell surface” or “associated with the cell surface” means that a molecule such as an antigen is associated with and located at the plasma membrane of a cell, wherein at least a part of the molecule faces the extracellular space of said cell and is accessible from the outside of said cell, e.g., by antibodies located outside the cell. In this context, a part is preferably at least 4, preferably at least 8, preferably at least 12, more preferably at least 20 amino acids. The association may be direct or indirect. For example, the association may be by one or more transmembrane domains, one or more lipid anchors, or by the interaction with any other protein, lipid, saccharide, or other structure that can be found on the outer leaflet of the plasma membrane of a cell. For example, a molecule associated with the surface of a cell may be a transmembrane protein having an extracellular portion or may be a protein associated with the surface of a cell by interacting with another protein that is a transmembrane protein.

“Cell surface” or “surface of a cell” is used in accordance with its normal meaning in the art, and thus includes the outside of the cell which is accessible to binding by proteins and other molecules. An antigen is expressed on the surface of cells if it is located at the surface of said cells and is accessible to binding by, e.g., antigen-specific antibodies added to the cells.

The term “extracellular portion” or “exodomain” in the context of the present disclosure refers to a part of a molecule such as a protein that is facing the extracellular space of a cell and preferably is accessible from the outside of said cell, e.g., by binding molecules such as antibodies located outside the cell. Preferably, the term refers to one or more extracellular loops or domains or a fragment thereof.

The terms “T cell” and “T lymphocyte” are used interchangeably herein and include T helper cells (CD4⁺ T cells) and cytotoxic T cells (CTLs, CD8⁺ T cells) which comprise cytolytic T cells. The term “antigen-specific T cell” or similar terms relate to a T cell which recognizes the antigen to which the T cell is targeted, in particular when presented on the surface of antigen presenting cells or diseased cells such as cancer cells in the context of MHC molecules and preferably exerts effector functions of T cells. T cells are considered to be specific for antigen if the cells kill target cells expressing an antigen. T cell specificity may be evaluated using any of a variety of standard techniques, for example, within a chromium release assay or proliferation assay. Alternatively, synthesis of lymphokines (such as interferon-γ) can be measured. In certain embodiments of the present disclosure, the RNA (in particular mRNA) encodes at least one epitope.

The term “target” shall mean an agent such as a cell or tissue which is a target for an immune response such as a cellular immune response. Targets include cells that present an antigen or an antigen epitope, i.e., a peptide fragment derived from an antigen. In one embodiment, the target cell is a cell expressing an antigen and preferably presenting said antigen with class I MHC.

“Antigen processing” refers to the degradation of an antigen into processing products which are fragments of said antigen (e.g., the degradation of a protein into peptides) and the association of one or more of these fragments (e.g., via binding) with MHC molecules for presentation by cells, preferably antigen-presenting cells to specific T-cells.

By “antigen-responsive CTL” is meant a CD8⁺ T-cell that is responsive to an antigen or a peptide derived from said antigen, which is presented with class I MHC on the surface of antigen presenting cells.

According to the present disclosure, CTL responsiveness may include sustained calcium flux, cell division, production of cytokines such as IFN-γ and TNF-α, up-regulation of activation markers such as CD44 and CD69, and specific cytolytic killing of tumor antigen expressing target cells. CTL responsiveness may also be determined using an artificial reporter that accurately indicates CTL responsiveness.

The terms “immune response” and “immune reaction” are used herein interchangeably in their conventional meaning and refer to an integrated bodily response to an antigen and preferably refers to a cellular immune response, a humoral immune response, or both. According to the present disclosure, the term “immune response to” or “immune response against” with respect to an agent such as an antigen, cell or tissue, relates to an immune response such as a cellular response directed against the agent. An immune response may comprise one or more reactions selected from the group consisting of developing antibodies against one or more antigens and expansion of antigen-specific T-lymphocytes, preferably CD4⁺ and CD8⁺ T-lymphocytes, more preferably CD8⁺ T-lymphocytes, which may be detected in various proliferation or cytokine production tests in vitro.

The terms “inducing an immune response” and “eliciting an immune response” and similar terms in the context of the present disclosure refer to the induction of an immune response, preferably the induction of a cellular immune response, a humoral immune response, or both. The immune response may be protective/preventive/prophylactic and/or therapeutic. The immune response may be directed against any immunogen or antigen or antigen peptide, preferably against a tumor-associated antigen or a pathogen-associated antigen (e.g., an antigen of a virus (such as influenza virus (A, B, or C), CMV or RSV)). “Inducing” in this context may mean that there was no immune response against a particular antigen or pathogen before induction, but it may also mean that there was a certain level of immune response against a particular antigen or pathogen before induction and after induction said immune response is enhanced. Thus, “inducing the immune response” in this context also includes “enhancing the immune response”. Preferably, after inducing an immune response in an individual, said individual is protected from developing a disease such as an infectious disease or a cancerous disease or the disease condition is ameliorated by inducing an immune response.

The terms “cellular immune response”, “cellular response”, “cell-mediated immunity” or similar terms are meant to include a cellular response directed to cells characterized by expression of an antigen and/or presentation of an antigen with class I or class II MHC. The cellular response relates to cells called T cells or T lymphocytes which act as either “helpers” or “killers”. The helper T cells (also termed CD4⁺ T cells) play a central role by regulating the immune response and the killer cells (also termed cytotoxic T cells, cytolytic T cells, CD8⁺ T cells or CTLs) kill cells such as diseased cells.

The term “humoral immune response” refers to a process in living organisms wherein antibodies are produced in response to agents and organisms, which they ultimately neutralize and/or eliminate. The specificity of the antibody response is mediated by T and/or B cells through membrane-associated receptors that bind antigen of a single specificity. Following binding of an appropriate antigen and receipt of various other activating signals, B lymphocytes divide, which produces memory B cells as well as antibody secreting plasma cell clones, each producing antibodies that recognize the identical antigenic epitope as was recognized by its antigen receptor. Memory B lymphocytes remain dormant until they are subsequently activated by their specific antigen. These lymphocytes provide the cellular basis of memory and the resulting escalation in antibody response when re-exposed to a specific antigen.

The terms “vaccination” and “immunization” describe the process of treating an individual for therapeutic or prophylactic reasons and relate to the procedure of administering one or more immunogen(s) or antigen(s) or derivatives thereof, in particular in the form of RNA (especially mRNA) coding therefor, as described herein to an individual and stimulating an immune response against said one or more immunogen(s) or antigen(s) or cells characterized by presentation of said one or more immunogen(s) or antigen(s).

By “cell characterized by presentation of an antigen” or “cell presenting an antigen” or “MHC molecules which present an antigen on the surface of an antigen presenting cell” or similar expressions is meant a cell such as a diseased cell, in particular a tumor cell or an infected cell, or an antigen presenting cell presenting the antigen or an antigen peptide, either directly or following processing, in the context of MHC molecules, preferably MHC class I and/or MHC class II molecules, most preferably MHC class I molecules.

In the context of the present disclosure, the term “transcription” relates to a process, wherein the genetic code in a DNA sequence is transcribed into RNA (especially mRNA). Subsequently, the RNA (especially mRNA) may be translated into peptide or protein.

With respect to RNA, the term “expression” or “translation” relates to the process in the ribosomes of a cell by which a strand of mRNA directs the assembly of a sequence of amino acids to make a peptide or protein.

The term “optional” or “optionally” as used herein means that the subsequently described event, circumstance or condition may or may not occur, and that the description includes instances where said event, circumstance, or condition occurs and instances in which it does not occur.

As used herein “endogenous” refers to any material from or produced inside an organism, cell, tissue or system.

The term “expression” as used herein is defined as the transcription and/or translation of a particular nucleotide sequence.

As used herein, the terms “linked”, “fused”, or “fusion” are used interchangeably. These terms refer to the joining together of two or more elements or components or domains.

The term “disease” (also referred to as “disorder” herein) refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g., mouse, rat, rabbit, dog, cat, cattle, swine, sheep, horse or primate), or any other non-mammal-animal, including birds (chicken), fish or any other animal species that can be afflicted with or is susceptible to a disease or disorder (e.g., cancer, infectious diseases) but may or may not have the disease or disorder, or may have a need for prophylactic intervention such as vaccination, or may have a need for interventions such as by protein replacement. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

### Aspects and Embodiments of the Present Disclosure

In a first aspect, the present disclosure provides a binding agent for use in a method for reducing or preventing progression of a tumor or treating cancer in a subject, said method comprising administering to said subject the binding agent in a suitable amount, wherein the binding agent comprises a first binding region binding to human CD40, such as human CD40 comprising the sequence set forth in SEQ ID NO: 36, and a second binding region binding to human CD137, such as human CD137 comprising the sequence set forth in SEQ ID NO: 38. For example, the method may comprise administering to said subject, in at least one treatment cycle, the binding agent in a suitable amount, wherein the binding agent comprises a first binding region binding to human CD40, such as human CD40 comprising the sequence set forth in SEQ ID NO: 36, and a second binding region binding to human CD137, such as human CD137 comprising the sequence set forth in SEQ ID NO: 38.

Preferably, the amount of binding agent administered, e.g., in each dose and/or treatment cycle, may induce intracellular signaling when binding to CD137 expressed on another cell. Thus, a binding agent in a suitable amount according to the present disclosure is able to trans-activate two different cells. In humans, CD40 is expressed on a number of cells including antigen-presenting cells (APCs), such as dendritic cells, whereas CD137 is expressed on T cells and other cells. Thus, a binding agent binding to CD40 and CD137 in a suitable amount according to the present disclosure is able to bind simultaneously to an APC and a T cell expressing these receptors. Without being bound by theory, a binding agent may thus (i) mediate cell-to-cell interaction between APCs and T cells by receptor binding and (ii) activate both CD40 and CD137 at once, which is primarily induced by cross-linking and receptor clustering upon cell-to-cell interaction and not necessarily dependent on agonistic activity of the parental monospecific bivalent antibodies. Thus, these trans-activating binding agent exert co-stimulatory activity in the context of APC:T cell interactions, and can elicit a T cell response against tumor cells. As such, this mechanism of action can reflect natural T-cell activation via antigen-presentation by activated APCs, allowing for the presentation of a variety of tumor-specific antigens by the APCs to T cells. Without being limited to theory, the costimulatory activity may provide for one or more of (i) only specific T cells being activated (i.e., those that are in contact with an APC) as opposed to any T cell; (ii) re-activation of exhausted T cells, by strong co-stimulation via activated APCs and CD137 triggering; and (iii) the priming of T cells by inducing antigen presentation by activated APCs and at the same time triggering CD137.

The dosing regimen of the binding agent disclosed herein (such as used in the methods disclosed herein) may vary depending upon the indication, route of administration, and severity of the condition, for example. Depending on the route of administration, a suitable dose can be calculated according to body weight, body surface area, or organ size. The final dosing regimen can be determined by the attending physician in view of good medical practice, considering various factors that modify the action of drugs, e.g., the specific activity of the binding agent, the disease to be treated, the disease state, the severity of disease, the individual parameters of the patient (including age, sex, physiological condition, diet, and weight of the patient), the specific route of administration, and the responsiveness of the subject. Additional factors that can be taken into account include time and frequency of administration, drug combinations, reaction sensitivities, and tolerance/response to therapy. Further refinement of the doses appropriate for treatment (involving in particular any of the compositions/formulations mentioned herein) can be done routinely by the skilled practitioner without undue experimentation, especially in light of the dosing information and assays disclosed herein, as well as the pharmacokinetic data observed in human clinical trials. Appropriate doses can be ascertained through use of established assays for determining concentration of the binding agent in a body fluid or other sample together with dose response data. The composition/formulation and route of administration chosen may be tailored to the individual subject, the nature of the disease to be treated in the subject, and generally, the judgment of the attending practitioner.

The amount of binding agent administered in each dose and/or treatment cycle may in particular be in a range, wherein more than 5%, preferably more than 10%, more preferably more than 15%, even more preferably more than 20%, even more preferably more than 25%, even more preferably more than 30%, even more preferably more than 35%, even more preferably more than 40%, even more preferably more than 45%, most preferably more than 50% of said binding agents bind to both, CD40 and CD137.

In preferred embodiments, the amount of binding agent administered, e.g., in each dose and/or in each treatment cycle, is
a) about 0.04-2.5 mg/kg body weight or about 3-200 mg in total; and/or
b) about 0.25×10⁻⁹-16.9×10⁻⁹ mol/kg body weight or about 20×10⁻⁹-1350×10⁻⁹ mol in total.

In one embodiment, the amount of binding agent administered, e.g., in each dose and/or in each treatment cycle, may be about 0.04-2.5 mg/kg body weight, such as about 0.06-1.25 mg/kg body weight, about 0.12-0.75 mg/kg body weight, or about 0.25-0.38 mg/kg body weight; or about 0.62-1.88 mg/kg body weight, about 0.93-1.56 mg/kg body weight, about 1.0-1.5 mg/kg body weight, or about 1.12-1.38 mg/kg body weight; or about 1.25 mg/kg body weight.

In one embodiment, the amount of binding agent administered, e.g., in each dose and/or in each treatment cycle, may be about 3-200 mg in total, such as about 5-100 mg, about 10⁻⁶⁰ mg, or about 20-30 mg in total; or about 50-150 mg, about 75-125 mg, about 80-120 mg, or about 90-110 mg in total; or about 100 mg in total.

In one embodiment, the amount of binding agent administered, e.g., in each dose and/or in each treatment cycle, may be about 0.25×10⁻⁹-16.9×10⁻⁹ mol/kg body weight, such as about 0.40×10⁻⁹-8.4×10⁻⁹ mol/kg body weight, about 0.81×10⁻⁹-5.1×10⁻⁹ mol/kg body weight, or about 1.69×10⁻⁹-−2.56×10⁻⁹ mol/kg body weight; or about 4.18×10⁻⁹-12.7×10⁻⁹ mol/kg body weight, about 6.28×10⁻⁹-10.5×10⁻⁹ mol/kg body weight, about 6.75×10⁻⁹-10.1×10⁻⁹ mol/kg body weight, or about 7.56×10⁻⁹-9.31×10⁻⁹ mol/kg body weight; or about 8.44×10⁻⁹ mol/kg body weight.

In one embodiment, the amount of binding agent administered, e.g., in each dose and/or in each treatment cycle, may be about 20×10⁻⁹-1350×10⁻⁹ mol in total, such as about 30×10⁻⁹-670×10⁻⁹ mol, about 60×10⁻⁹-410×10⁻⁹ mol, or about 135×10⁻⁹-205×10⁻⁹ mol in total; or about 330×10⁻⁹-1020×10⁻⁹ mol, about 500×10⁻⁹-840×10⁻⁹ mol, about 540×10⁻⁹-810×10⁻⁹ mol, or about 600×10⁻⁹-745×10⁻⁹ mol in total; or about 675×10⁻⁹ mol in total.

According to these embodiments, the dose defined in mg/kg may be converted to flat dose, and vice versa, based on the median body weight of the subjects to whom the binding agent is administered being 80 kg. Further, the amount of binding agent administered, e.g., in each dose and/or in each treatment cycle, may in particular be
about 0.62-1.88 mg/kg body weight (such as about 0.93-1.56 mg/kg body weight, about 1.0-1.5 mg/kg body weight, or about 1.12-1.38 mg/kg body weight, or about 1.25 mg/kg body weight) or about 50-150 mg (such as about 75-125 mg, about 80-120 mg, or about 90-110 mg, or about 100 mg) in total; and/or
about 4.18×10⁻⁹-12.7×10⁻⁹ mol/kg body weight (such as about 6.28×10⁻⁹-10.5×10⁻⁹ mol/kg body weight, about 6.75×10⁻⁹-10.1×10⁻⁹ mol/kg body weight, or about 7.56×10⁻⁹-9.31×10⁻⁹ mol/kg body weight, or about 8.44×10⁻⁹ mol/kg body weight) or about 330×10⁻⁹-1020×10⁻⁹ mol (such as about 500×10⁻⁹-840×10⁻⁹ mol, about 540×10⁻⁹-810×10⁻⁹ mol, or about 600×10⁻⁹-745×10⁻⁹ mol, or about 675×10⁻⁹ mol) in total.

In one embodiment of the binding agent according to the first aspect,
a) the first binding region binding to human CD40 comprises a heavy chain variable region (VH) comprising the CDR1, CDR2, and CDR3 sequences of SEQ ID NO: 7 or 9, and a light chain variable region (VL) comprising the CDR1, CDR2, and CDR3 sequences of SEQ ID NO: 8 or 10;
and
b) the second antigen-binding region binding to human CD137 comprises a heavy chain variable region (VH) comprising the CDR1, CDR2, and CDR3 sequences of SEQ ID NO: 17 or 19, and a light chain variable region (VL) comprising the CDR1, CDR2, and CDR3 sequences of SEQ ID NO: 18 or 20.

In one embodiment of the binding agent according to the first aspect,
a) the first binding region binding to human CD40 comprises a heavy chain variable region (VH) comprising the CDR1, CDR2, and CDR3 sequences set forth in: SEQ ID NO: 1, 2, and 3, respectively, and a light chain variable region (VL) comprising the CDR1, CDR2, and CDR3 sequences set forth in: SEQ ID NO: 4, 5, and 6, respectively;
and
b) the second antigen-binding region binding to human CD137 comprises a heavy chain variable region (VH) comprising the CDR1, CDR2, and CDR3 sequences set forth in: SEQ ID NO: 11, 12, and 13, respectively, and a light chain variable region (VL) comprising the CDR1, CDR2, and CDR3 sequences set forth in: SEQ ID NO: 14, 15, and 16, respectively.

In one embodiment of the binding agent according to the first aspect,
a) the first binding region binding to human CD40 comprises a heavy chain variable region (VH) comprising an amino acid sequence having at least 90%, at least 95%, at least 97%, at least 99%, or 100% sequence identity to SEQ ID NO: 7 or 9 and a light chain variable region (VL) region and comprising an amino acid sequence having at least 90%, at least 95%, at least 97%, at least 99%, or 100% sequence identity to SEQ ID NO: 8 or 10;
b) the second binding region binding to human CD137 comprises a heavy chain variable region (VH) comprising an amino acid sequence having at least 90%, at least 95%, at least 97%, at least 99%, or 25 100% sequence identity to SEQ ID NO: 17 or 19 and a light chain variable region (VL) region comprising an amino acid sequence having at least 90%, at least 95%, at least 97%, at least 99%, or 100% sequence identity to SEQ ID NO: 18 or 20.

In one embodiment of the binding agent according to the first aspect,
a) the first binding region binding to human CD40 comprises a heavy chain variable region (VH) comprising the amino acid sequence set forth in SEQ ID NO: 7 or 9 and a light chain variable region (VL) region comprising the amino acid sequence set forth in SEQ ID NO: 8 or 10;
and
b) the second binding region binding to human CD137 comprises a heavy chain variable region (VH) comprising the amino acid sequence set forth in SEQ ID NO: 17 or 19 and a light chain variable region (VL) region comprising the amino acid sequence set forth in SEQ ID NO: 18 or 20.

In one embodiment of the binding agent according to the first aspect,
a) the first binding region binding to human CD40 comprises a heavy chain variable region (VH) comprising the amino acid sequence set forth in SEQ ID NO: 9 and a light chain variable region (VL) region comprising the amino acid sequence set forth in SEQ ID NO: 10;
and
b) the second binding region binding to human CD137 comprises a heavy chain variable region (VH) comprising the amino acid sequence set forth in SEQ ID NO: 19 and a light chain variable region (VL) region comprising the amino acid sequence set forth in SEQ ID NO: 20.

The binding agent may in particular be an antibody, such as a multispecific antibody, e.g., a bispecific antibody. Also, the binding agent may be in the format of a full-length antibody or an antibody fragment.

It is further preferred that the antibody is a human antibody or a humanized antibody.

Each variable region may comprise three complementarity determining regions (CDR1, CDR2, and CDR3) and four framework regions (FR1, FR2, FR3, and FR4).

The complementarity determining regions (CDRs) and the framework regions (FRs) may be arranged from amino-terminus to carboxy-terminus in the following order: FR1, CDR1, FR2, CDR2, FR3, CDR3, FR4.

In one embodiment of the first aspect, the binding agent comprises
i) a polypeptide comprising said first heavy chain variable region (VH) and a first heavy chain constant region (CH), and
ii) a polypeptide comprising said second heavy chain variable region (VH) and a second heavy chain constant region (CH).

In one embodiment of the first aspect, the binding agent comprises
i) a polypeptide comprising said first light chain variable region (VL) and further comprising a first light chain constant region (CL), and
ii) a polypeptide comprising said second light chain variable region (VL) and further comprising a second light chain constant region (CL).

In one embodiment of the first aspect, the binding agent is an antibody comprising a first binding arm and a second binding arm, wherein the first binding arm comprises
i) a polypeptide comprising said first heavy chain variable region (VH) and said first heavy chain constant region (CH), and
ii) a polypeptide comprising said first light chain variable region (VL) and said first light chain constant region (CL);
and the second binding arm comprises
iii) a polypeptide comprising said second heavy chain variable region (VH) and said second heavy chain constant region (CH), and
iv) a polypeptide comprising said second light chain variable region (VL) and said second light chain constant region (CL).

Each of the first and second heavy chain constant regions (CH) may comprise one or more of a constant heavy chain 1 (CH1) region, a hinge region, a constant heavy chain 2 (CH2) region and a constant heavy chain 3 (CH3) region, preferably at least a hinge region, a CH2 region and a CH3 region.

Each of the first and second heavy chain constant regions (CHs) may comprise a CH3 region, wherein the two CH3 regions comprise asymmetrical mutations. Asymmetrical mutations mean that the sequences of said first and second CH3 regions contain amino acid substitutions at non-identical positions. For example, one of said first and second CH3 regions contains a mutation at the position corresponding to position 405 in a human IgG1 heavy chain according to EU numbering, and the other of said first and second CH3 regions contains a mutation at the position corresponding to position 409 in a human IgG1 heavy chain according to EU numbering.

In said first heavy chain constant region (CH) at least one of the amino acids in a position corresponding to a position selected from the group consisting of T366, L368, K370, D399, F405, Y407, and K409 in a human IgG1 heavy chain according to EU numbering may have been substituted, and in said second heavy chain constant region (CH) at least one of the amino acids in a position corresponding to a position selected from the group consisting of T366, L368, K370, D399, F405, Y407, and K409 in a human IgG1 heavy chain according to EU numbering may have been substituted. In particular embodiments, the first and the second heavy chains are not substituted in the same positions (i.e., the first and the second heavy chains contain asymmetrical mutations).

In one embodiment of the binding agent according to the first aspect, (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.

In one embodiment of the first aspect, the binding agent induces Fc-mediated effector function to a lesser extent compared to another antibody comprising the same first and second antigen binding regions and two heavy chain constant regions (CHs) comprising human IgG1 hinge, CH2 and CH3 regions.

In one particular embodiment of the binding agent according to the first aspect, said first and second heavy chain constant regions (CHs) are modified so that the antibody induces Fc-mediated effector function to a lesser extent compared to an antibody which is identical except for comprising non-modified first and second heavy chain constant regions (CHs). In particular, each or both of said non-modified first and second heavy chain constant regions (CHs) may comprise, consists of or consist essentially of the amino acid sequence set forth in SEQ ID NO: 21 or 29.

The Fc-mediated effector function may be determined by measuring binding of the binding agent to Fcγ receptors, binding to C1q, or induction of Fc-mediated cross-linking of Fcγ receptors. In particular, the Fc-mediated effector function may be determined by measuring binding of the binding agent to C1q. The first and second heavy chain constant regions of the binding agent may have been modified so that binding of C1q to said antibody is reduced compared to a wild-type antibody, preferably reduced by at least 70%, at least 80%, at least 90%, at least 95%, at least 97%, or 100%, wherein C1q binding is preferably determined by ELISA.

In one embodiment of the binding agent according to the first aspect, in at least one of said first and second heavy chain constant regions (CH), one or more amino acids in the positions corresponding to positions L234, L235, D265, N297, and P331 in a human IgG1 heavy chain according to EU numbering, are not L, L, D, N, and P, respectively.

In one embodiment of the binding agent according to the first aspect, the positions corresponding to positions L234 and L235 in a human IgG1 heavy chain according to EU numbering may be F and E, respectively, in said first and second heavy chains.

In particular, the positions corresponding to positions L234, L235, and D265 in a human IgG1 heavy chain according to EU numbering may be F, E, and A, respectively, in said first and second heavy chain constant regions (HCs).

In one embodiment of the binding agent according to the first aspect, the positions corresponding to positions L234 and L235 in a human IgG1 heavy chain according to EU numbering of both the first and second heavy chain constant regions are F and E, respectively, wherein (i) the position corresponding to F405 in a human IgG1 heavy chain according to EU numbering of the first heavy chain constant region is L, and the position corresponding to K409 in a human IgG1 heavy chain according to EU numbering of the second heavy chain is R, or (ii) the position corresponding to K409 in a human IgG1 heavy chain according to EU numbering of the first heavy chain constant region is R, and the position corresponding to F405 in a human IgG1 heavy chain according to EU numbering of the second heavy chain is L.

In one embodiment of the binding agent according to the first aspect, the positions corresponding to positions L234, L235, and D265 in a human IgG1 heavy chain according to EU numbering of both the first and second heavy chain constant regions are F, E, and A, respectively, wherein (i) the position corresponding to F405 in a human IgG1 heavy chain according to EU numbering of the first heavy chain constant region is L, and the position corresponding to K409 in a human IgG1 heavy chain according to EU numbering of the second heavy chain constant region is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.

In one embodiment of the binding agent according to the first aspect, the constant region of said first and/or second heavy chain comprises an amino acid sequence selected from the group consisting of
a) the sequence set forth in SEQ ID NO: 21 or SEQ ID NO: 29 [IgG1-FC];
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4, at the most 3, at the most 2 or at the most 1 substitution compared to the amino acid sequence defined in a) or b).

In one embodiment of the binding agent according to the first aspect, the constant region of said first or second heavy chain, such as the second heavy chain, comprises or consists essentially of or consists of an amino acid sequence selected from the group consisting of
a) the sequence set forth in SEQ ID NO: 22 or SEQ ID NO: 30 [IgG1-F405L];
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 9 substitutions, such as at the most 8, at the most 7, at the most 6, at the most 5, at the most 4, at the most 3, at the most 2 or at the most 1 substitution compared to the amino acid sequence defined in a) or b).

In one embodiment of the binding agent according to the first aspect, the constant region of said first or second heavy chain, such as the first heavy chain comprises or consists essentially of or consists of an amino acid sequence selected from the group consisting of
a) the sequence set forth in SEQ ID NO: 23 or 31 [IgG1-F409R];
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution compared to the amino acid sequence defined in a) or b).

In one embodiment of the binding agent according to the first aspect, the constant region of said first and/or second heavy chain comprises or consists essentially of or consists of an amino acid sequence selected from the group consisting of
a) the sequence set forth in SEQ ID NO: 24 or SEQ ID NO: 32 [IgG1-Fc_FEA];
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 7 substitutions, such as at the most 6 substitutions, at the most 5, at the most 4, at the most 3, at the most 2 or at the most 1 substitution compared to the amino acid sequence defined in a) or b).

In one embodiment of the binding agent according to the first aspect, the constant region of said first and/or second heavy chain, such as the second heavy chain, comprises or consists essentially of or consists of an amino acid sequence selected from the group consisting of
a) the sequence set forth in SEQ ID NO: 25 or SEQ ID NO: 33 [IgG1-Fc_FEAL];
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 6 substitutions, such as at the most 5 substitutions, at the most 4 substitutions, at the most 3, at the most 2 or at the most 1 substitution compared to the amino acid sequence defined in a) or b).

In one embodiment of the binding agent according to the first aspect, the constant region of said first and/or second heavy chain, such as the first heavy chain, comprises or consists essentially of or consists of an amino acid sequence selected from the group consisting of
a) the sequence set forth in SEQ ID NO: 26 or SEQ ID NO: 34 [IgG1-Fc_FEAR];
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 6 substitutions, such as at the most 5 substitutions, at the most 4, at the most 3, at the most 2 or at the most 1 substitution compared to the amino acid sequence defined in a) or b).

In one embodiment of the first aspect, the binding agent comprises a kappa (κ) light chain constant region.

In one embodiment of the first aspect, the binding agent comprises a lambda (λ) light chain constant region.

In one embodiment of the binding agent according to the first aspect, the first light chain constant region is a kappa (κ) light chain constant region or a lambda (λ) light chain constant region.

In one embodiment of the binding agent according to the first aspect, the second light chain constant region is a lambda (λ) light chain constant region or a kappa (κ) light chain constant region.

In one embodiment of the binding agent according to the first aspect, the first light chain constant region is a kappa (κ) light chain constant region and the second light chain constant region is a lambda (λ) light chain constant region or the first light chain constant region is a lambda (λ) light chain constant region and the second light chain constant region is a kappa (κ) light chain constant region.

In one embodiment of the binding agent according to the first aspect, the kappa (κ) light chain comprises an amino acid sequence selected from the group consisting of
a) the sequence set forth in SEQ ID NO: 27;
b) a subsequence of the sequence in a), such as a subsequence wherein 1, 2, 3, 4, 5, 6, 7, 8, 9 or 10 consecutive amino acids has/have been deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution, compared to the amino acid sequence defined in a) or b).

In one embodiment of the binding agent according to the first aspect, the lambda (λ) light chain comprises an amino acid sequence selected from the group consisting of
a) the sequence set forth in SEQ ID NO: 28;
b) a subsequence of the sequence in a), such as a subsequence wherein 1, 2, 3, 4, 5, 6, 7, 8, 9 or 10 consecutive amino acids has/have been deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution, compared to the amino acid sequence defined in a) or b).

The binding agent (in particular, antibody) according to the first aspect is of an isotype selected from the group consisting of IgG1, IgG2, IgG3, and IgG4. In particular, the binding agent may be a full-length IgG1 antibody. In preferred embodiments of the first aspect, the binding agent (in particular, antibody) is of the IgG1m(f) allotype.

In further preferred embodiments, the binding agent is Tecaginlimab, or a biosimilar thereof.

The subject to be treated according to the present disclosure is preferably a human subject.

In particular embodiments, the tumor or cancer is selected from the group consisting of melanoma, lung cancer, and colorectal cancer.

The tumor or cancer may in particular be a melanoma. Melanoma is the 19^{th} most common malignancy with an estimated age-standardized incidence rate of 3.0 per 100,000 (Ferlay et al., 2015). Worldwide, approximately 287,700 new cases of melanoma and 60,700 deaths were estimated for 2018 (Ferlay et al., 2018). In the US, approximately 91,270 new cases of melanoma and about 9,320 deaths were estimated for 2018. As with almost all malignancies, the outcome of melanoma depends on the stage at presentation. There is an increasing appreciation of the variations in specific genetic alterations among distinct clinical subtypes of melanoma, some of which have different therapeutic implications (NCCN, 2018a). Therefore, the choice for first-line therapy is based on the evaluation of the individual patient.

Standard of care for patients with advanced or metastatic melanoma who have progressed on targeted therapy or immunotherapy may receive high-dose interleukin (IL)-2 or other cytotoxic therapies (e.g., dacarbazine, carboplatin/paclitaxel, albumin-bound paclitaxel, etc.). These agents have modest response rates of less than 20% in the first-line and second-line settings and little consensus exists regarding optimal standard chemotherapy (NCCN 2018a).

In one embodiment, the melanoma cancer is cutaneous, acral, or mucosal melanoma.

In one embodiment, wherein the tumor or cancer is melanoma, the subject has received up to four prior systemic treatments for advanced/metastatic disease and has experienced disease progression on or after last prior systemic treatment, such as disease progression determined by radiography.

In one embodiment, wherein the tumor or cancer is melanoma, the subject has received prior treatment with checkpoint inhibitor(s), such as agent(s) targeting PD-1/PD-L, such as a PD-1/PD-L1 inhibitor.

The tumor or cancer may in particular be a colorectal cancer (CRC). CRC is the third most commonly diagnosed cancer in males and the second in females. Worldwide, approximately 1,096,600 new cases of CRC and 551,300 deaths were estimated for 2018 (Ferlay et al., 2018). In the US, 140,250 new cases of CRC and 50,630 deaths due to CRC were estimated for 2018. Five-year relative survival rates in the US were 71% for patients with regional disease at diagnosis and only 14% for patients with distant disease at diagnosis (rates are adjusted for normal life expectancy and are based on cases diagnosed in the SEER 18 areas from 2008-2014 (SEER, 2018).

The current management of metastatic CRC (mCRC) involves various drugs, either in combination or as single agents. Choice of therapy is influenced by type and timing of prior therapy, therapy goals, mutational profile of the tumor, and the toxicity profile of the constituent drugs (NCCN 2018b). Recommended initial therapy options for advanced or metastatic disease depend on whether the patient is appropriate for intensive therapy. The more intensive initial therapy options include FOLFOX, FOLFIRI, CapeOx, and FOLFOXIRI. Addition of a biological agent (e.g., bevacizumab, cetuximab, panitumumab) is also an option in combination with some of these regimens. Systemic therapy options for patients with progressive disease (PD) depend on the choice of initial therapy.

In one embodiment, wherein the tumor or cancer is CRC, the subject has received up to four prior systemic treatments for advanced/metastatic disease and has experienced disease progression on or after last prior systemic treatment, such as disease progression determined by radiography.

In one embodiment, wherein the tumor or cancer is CRC, the subject has received 5-fluorouracil (FU)-based therapy.

In one embodiment, wherein the tumor or cancer is CRC, the subject has not received treatment with an immune checkpoint (ICP) inhibitor.

The tumor or cancer may in particular be a lung cancer. The lung cancer may be a non-small cell lung cancer (NSCLC), such as a squamous or a non-squamous NSCLC. Lung cancer is the most common malignancy and the most common cause of cancer death worldwide. Non-small cell lung cancer (NSCLC) accounts for 85-90% of all lung cancer cases (Jemal et al., 2011). The five-year survival rate for NSCLC is approximately 18% (SEER, 2018). Major histological subtypes of NSCLC include adenocarcinoma, squamous cell carcinoma, adenosquamous carcinoma, large cell carcinoma, carcinoid tumors, and other less common subtypes, with adenocarcinoma being the most common.

Standard of care for patients with advanced or metastatic NSCLC who have progressed on targeted therapy or are no longer candidates for targeted therapy typically includes platinum-based chemotherapy. Platinum combinations have generated an overall response rate (ORR) of approximately 25-35%, a time to progression (TTP) of 4-6 months, and median survival of 8-10 months.

In one embodiment, the lung cancer is a NSCLC, such as a squamous or non-squamous NSCLC. In one embodiment, the NSCLC does not have an epidermal growth factor (EGFR)-sensitizing mutation and/or anaplastic lymphoma (ALK) translocation/c-ROS oncogene 1 (ROS1) rearrangement.

In one embodiment, wherein the tumor or cancer is lung cancer, the subject has received up to four prior systemic treatment regimens for advanced/metastatic disease and has experienced disease progression on or after last prior systemic treatment, such as disease progression determined by radiography. For example, the subject has received platinum-based chemotherapy. Alternatively, the subject is not eligible for platinum-based therapy and has received alternative chemotherapy, e.g., a treatment with gemcitabine-containing regimen.

In one embodiment, wherein the tumor or cancer is lung cancer, the subject has received prior treatment with checkpoint inhibitor(s), such as agent(s) targeting PD-1/PD-L, such as a PD-1/PD-L1 inhibitor.

In one embodiment, wherein the tumor or cancer is lung cancer, the subject has experienced disease progression on or after last prior systemic treatment, such as disease progression determined by radiography.

In one embodiment of the first aspect, the binding agent is in particular administered to said subject by systemic administration. Preferably, the binding agent is administered to said subject by intravenous injection or infusion.

In one embodiment of the first aspect, each treatment cycle is about two weeks (14 days), three weeks (21 days) or four weeks (28 days), preferably three weeks (21 days).

In particular embodiments, each dose is administered or infused every second week (1Q2W), every third week (1Q3W) or every fourth week (1Q4W), preferably every third week (1Q3W).

In some embodiments, one dose or each dose is administered or infused on day 1 of each treatment cycle.

Each dose may be administered or infused over a minimum of 30 minutes, such as over a minimum of 60 minutes, a minimum of 90 minutes, a minimum of 120 minutes or a minimum of 240 minutes.

In a second aspect, the present disclosure relates to a composition, such as a pharmaceutical composition, comprising a binding agent comprising a first binding region binding to human CD40 and a second binding region binding to human CD137, wherein the amount of binding agent in the composition is about 3-200 mg (such as about 5-100 mg, about 10-60 mg, or about 20-30 mg; or about 50-150 mg, about 75-125 mg, about 80-120 mg, or about 90-110 mg; or about 100 mg) or about 20×10⁻⁹-1350×10⁻⁹ mol (such as about 30×10⁻⁹-670×10⁻⁹ mol, about 60×10⁻⁹-410×10⁻⁹ mol, or about 135×10⁻⁹-205×10⁻⁹ mol; or about 330×10⁻⁹-1020×10⁻⁹ mol, about 500×10⁻⁹-840×10⁻⁹ mol, about 540×10⁻⁹-810×10⁻⁹ mol, or about 600×10⁻⁹-745×10⁻⁹ mol; or about 675×10⁻⁹ mol).

The amount of binding agent administered in said composition may in particular be about 0.04-2.5 mg/kg body weight (such as about 0.06-1.25 mg/kg body weight, about 0.12-0.75 mg/kg body weight, or about 0.25-0.38 mg/kg body weight; or about 0.62-1.88 mg/kg body weight, about 0.93-1.56 mg/kg body weight, about 1.0-1.5 mg/kg body weight, or about 1.12-1.38 mg/kg body weight; or about 1.25 mg/kg body weight) or about 3-200 mg (such as about 5-100 mg, about 10⁻⁶⁰ mg, or about 20-30 mg; or about 50-150 mg, about 75-125 mg, about 80-120 mg, or about 90-110 mg; or about 100 mg) in total; and/or about 0.25×10⁻⁹-16.9×10⁻⁹ mol/kg body weight (such as about 0.40×10⁻⁹-8.4×10⁻⁹ mol/kg body weight, about 0.81×10⁻⁹-5.1×10⁻⁹ mol/kg body weight, or about 1.69×10⁻⁹-−2.56×10⁻⁹ mol/kg body weight; or about 4.18×10⁻⁹-12.7×10⁻⁹ mol/kg body weight, about 6.28×10⁻⁹-10.5×10⁻⁹ mol/kg body weight, about 6.75×10⁻⁹-10.1×10⁻⁹ mol/kg body weight, or about 7.56×10⁻⁹-9.31×10⁻⁹ mol/kg body weight; or about 8.44×10⁻⁹ mol/kg body weight) or about 20×10⁻⁹-1350×10⁻⁹ mol (such as about 30×10⁻⁹-670×10⁻⁹ mol, about 60×10⁻⁹-410×10⁻⁹ mol, or about 135×10⁻⁹-205×10⁻⁹ mol; or about 330×10⁻⁹-1020×10⁻⁹ mol, about 500×10⁻⁹-840×10⁻⁹ mol, about 540×10⁻⁹-810×10⁻⁹ mol, or about 600×10⁻⁹-745×10⁻⁹ mol, or about 675×10⁻⁹ mol) in total.

The embodiments described above for the first aspect also apply to the second aspect. Thus, e.g., the binding agent contained in the composition of the second aspect may be any binding agent defined for the first aspect.

The composition or pharmaceutical composition may be formulated with a carrier, excipient and/or diluent as well as any other components suitable for pharmaceutical compositions, including known adjuvants, in accordance with conventional techniques such as those disclosed in Remington: The Science and Practice of Pharmacy, 19^{th} Edition, Gennaro, Ed., Mack Publishing Co., Easton, P A, 1995. The pharmaceutically acceptable carriers or diluents as well as any known adjuvants and excipients should be suitable for the binding agent of the present disclosure and the chosen mode of administration. Suitability for carriers and other components of pharmaceutical compositions is determined based on the lack of significant negative impact on the desired biological properties of the chosen compound or pharmaceutical composition of the second aspect (e.g., less than a substantial impact [10% or less relative inhibition, 5% or less relative inhibition, etc.] upon antigen binding).

The composition of the second aspect, in particular the pharmaceutical composition of the second aspect, may include diluents, fillers, salts, buffers, detergents (e.g., a nonionic detergent, such as Tween-20 or Tween-80), stabilizers (e.g., sugars or protein-free amino acids), preservatives, solubilizers, and/or other materials suitable for inclusion in a pharmaceutical composition.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

Pharmaceutically acceptable carriers include any and all suitable solvents, dispersion media, coatings, antibacterial and antifungal agents, isotonicity agents, antioxidants and absorption-delaying agents, and the like that are physiologically compatible with the active compound, in particular a binding agent as used herein.

Examples of suitable aqueous and non-aqueous carriers which may be employed in the (pharmaceutical) compositions of the second aspect include water, saline, phosphate buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the (pharmaceutical) compositions of the second aspect is contemplated.

The term “excipient” as used herein refers to a substance which may be present in a (pharmaceutical) composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water

A (pharmaceutical) composition of the second aspect may also comprise pharmaceutically acceptable antioxidants for instance (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

A (pharmaceutical) composition of the second aspect may also comprise isotonicity agents, such as sugars, polyalcohols, such as mannitol, sorbitol, glycerol or sodium chloride in the composition.

A (pharmaceutical) composition of the second aspect may also contain one or more adjuvants appropriate for the chosen route of administration such as preservatives, wetting agents, emulsifying agents, dispersing agents, preservatives or buffers, which may enhance the shelf life or effectiveness of the composition. The combination of binding agents as used herein may be prepared with carriers that will protect the compound against rapid release, such as a controlled release formulation, including implants, transdermal patches, and micro-encapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, poly-ortho esters, and polylactic acid alone or with a wax, or other materials well known in the art. Methods for the preparation of such formulations are generally known to those skilled in the art, see e.g. Sustained and Controlled Release Drug Delivery Systems, J. R. Robinson, ed., Marcel Dekker, Inc., New York, 1978.

“Pharmaceutically acceptable salts” comprise, for example, acid addition salts which may, for example, be formed by using a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, suitable pharmaceutically acceptable salts may include alkali metal salts (e.g., sodium or potassium salts); alkaline earth metal salts (e.g., calcium or magnesium salts); ammonium (NH₄); and salts formed with suitable organic ligands (e.g., quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include, but are not limited to, acetate, adipate, alginate, arginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, galactate, galacturonate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isobutyrate, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, phthalate, picrate, pivalate, polygalacturonate, propionate, salicylate, stearate, sulfate, suberate, succinate, tannate, tartrate, teoclate, tosylate, triethiodide, undecanoate, valerate, and the like (see, for example, S. M. Berge et al., “Pharmaceutical Salts”, J. Pharm. Sci., 66, pp. 1-19 (1977)). Salts which are not pharmaceutically acceptable may be used for preparing pharmaceutically acceptable salts and are included in the present disclosure.

In one embodiment, the binding agent used herein may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art. Except in so far as any conventional media or agent is incompatible with the active compound, use thereof in the compositions of the second aspect is contemplated. Other active or therapeutic compounds may also be incorporated into the compositions.

Pharmaceutical compositions for injection must typically be sterile and stable under the conditions of manufacture and storage. The composition may be formulated as a solution, micro-emulsion, liposome, or other ordered structure suitable to high drug concentration. The carrier may be an aqueous or a non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients e.g. from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Sterile injectable solutions may be prepared by incorporating the active compounds in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, examples of methods of preparation are vacuum-drying and freeze-drying (lyophilization) that yield a powder of the active ingredient plus any additional desired ingredient from a previously sterile-filtered solution thereof.

In a third aspect, the present disclosure provides a composition of the second aspect for use in a method for reducing or preventing progression of a tumor or treating cancer in a subject. The embodiments described above for the first and second aspect also apply to the third aspect. E.g., the binding agent contained in the composition of the third aspect may be any binding agent defined in the first aspect. In addition, the composition used in the third aspect may be any composition defined in the second aspect. Furthermore, the subject, cancer or tumor referred to in the third aspect may be any subject, cancer or tumor defined in the first aspect.

In a fourth aspect, the present disclosure provides a method for reducing or preventing progression of a tumor or treating cancer in a subject, said method comprising administering to said subject a binding agent in a suitable amount, wherein the binding agent comprises a first binding region binding to human CD40, such as human CD40 comprising the sequence set forth in SEQ ID NO: 36, and a second binding region binding to human CD137, such as human CD137 comprising the sequence set forth in SEQ ID NO: 38. The embodiments described above for the first aspect also apply to the fourth aspect. Thus, e.g., the binding agent used in the fourth aspect may be any binding agent defined in the first aspect. Furthermore, the subject, cancer or tumor referred to in the fourth aspect may be any subject, cancer or tumor defined in the first aspect.

In a fifth aspect, the present disclosure provides a method for reducing or preventing progression of a tumor or treating cancer in a subject, said method comprising administering to said subject a composition comprising binding agent in an amount between about 3-200 mg (such as about 5-100 mg, about 10⁻⁶⁰ mg, or about 20-30 mg; or about 50-150 mg, about 75-125 mg, about 80-120 mg, or about 90-110 mg; or about 100 mg) or about 20×10⁻⁹-1350×10⁻⁹ mol (such as about 0.40×10⁻⁹-8.4×10⁻⁹ mol/kg body weight, about 0.81×10⁻⁹-5.1×10⁻⁹ mol/kg body weight, or about 1.69×10⁻⁹-−2.56×10⁻⁹ mol/kg body weight; or about 4.18×10⁻⁹-12.7×10⁻⁹ mol/kg body weight, about 6.28×10⁻⁹-10.5×10⁻⁹ mol/kg body weight, about 6.75×10⁻⁹-10.1×10⁻⁹ mol/kg body weight, or about 7.56×10⁻⁹-9.31×10⁻⁹ mol/kg body weight; or about 8.44×10⁻⁹ mol/kg body weight) or about 20×10⁻⁹-1350×10⁻⁹ mol (such as about 30×10⁻⁹-670×10⁻⁹ mol, about 60×10⁻⁹-410×10⁻⁹ mol, or about 135×10⁻⁹-205×10⁻⁹ mol; or about 330×10⁻⁹-1020×10⁻⁹ mol, about 500×10⁻⁹-840×10⁻⁹ mol, about 540×10⁻⁹-810×10⁻⁹ mol, or about 600×10⁻⁹-745×10⁻⁹ mol, or about 675×10⁻⁹ mol), wherein the binding agent comprises a first binding region binding to human CD40, such as human CD40 comprising the sequence set forth in SEQ ID NO: 36, and a second binding region binding to human CD137, such as human CD137 comprising the sequence set forth in SEQ ID NO: 38. The embodiments described above for the first and second aspect also apply to the fourth aspect. E.g., the binding agent contained in the composition of the fifth aspect may be any binding agent defined in the first aspect. In addition, the composition used in the fifth aspect may be any composition defined in the second aspect. Furthermore, the subject, cancer or tumor referred to in the third aspect may be any subject, cancer or tumor defined in the first aspect.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The description (including the following examples) is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Additional items of the present disclosure include:
1. A binding agent for use in a method for reducing or preventing progression of a tumor or treating cancer in a subject, said method comprising administering to said subject the binding agent in a suitable amount, wherein the binding agent comprises a first binding region binding to human CD40, such as human CD40 comprising the sequence set forth in SEQ ID NO: 36, and a second binding region binding to human CD137, such as human CD137 comprising the sequence set forth in SEQ ID NO: 38.
2. The binding agent for use of item 1, wherein the suitable amount of the binding agent is a therapeutically effective and safe amount.
3. The binding agent for use of any one of the preceding items, wherein the suitable amount of the binding agent is about 0.04-2.5 mg/kg body weight or about 3-200 mg in total; and/or about 0.25×10⁻⁹-16.9×10⁻⁹ mol/kg body weight or about 20×10⁻⁹-1350×10⁻⁹ mol in total.
4. The binding agent for use of any one of the preceding items, wherein the binding agent is administered systemically, preferably intravenously.
5. The binding agent for use of any one of the preceding items, wherein
a) the first binding region comprises a heavy chain variable region (VH) comprising the CDR1, CDR2, and CDR3 sequences of SEQ ID NO: 7 or 9, and a light chain variable region (VL) comprising the CDR1, CDR2, and CDR3 sequences of SEQ ID NO: 8 or 10;
and
b) the second antigen-binding region comprises a heavy chain variable region (VH) comprising the CDR1, CDR2, and CDR3 sequences of SEQ ID NO: 17 or 19, and a light chain variable region (VL) comprising the CDR1, CDR2, and CDR3 sequences of SEQ ID NO: 18 or 20.
6. The binding agent for use of any one of the preceding items, wherein
a) the first binding region comprises a heavy chain variable region (VH) comprising the CDR1, CDR2, and CDR3 sequences set forth in: SEQ ID NO: 1, 2, and 3, respectively, and a light chain variable region (VL) comprising the CDR1, CDR2, and CDR3 sequences set forth in: SEQ ID NO: 4, 5, and 6, respectively;
and
b) the second antigen-binding region comprises a heavy chain variable region (VH) comprising the CDR1, CDR2, and CDR3 sequences set forth in: SEQ ID NO: 11, 12, and 13, respectively, and a light chain variable region (VL) comprising the CDR1, CDR2, and CDR3 sequences set forth in: SEQ ID NO: 14, 15, and 16, respectively.
7. The binding agent for use of any one of the preceding items, wherein
a) the first binding region comprises a heavy chain variable region (VH) comprising an amino acid sequence having at least 90%, at least 95%, at least 97%, at least 99%, or 100% sequence identity to SEQ ID NO: 7 or 9 and a light chain variable region (VL) region and comprising an amino acid sequence having at least 90%, at least 95%, at least 97%, at least 99%, or 100% sequence identity to SEQ ID NO: 8 or 10;
b) the second binding region comprises a heavy chain variable region (VH) comprising an amino acid sequence having at least 90%, at least 95%, at least 97%, at least 99%, or 25 100% sequence identity to SEQ ID NO: 17 or 19 and a light chain variable region (VL) region comprising an amino acid sequence having at least 90%, at least 95%, at least 97%, at least 99%, or 100% sequence identity to SEQ ID NO: 18 or 20.
8. The binding agent for use of any one of the preceding items, wherein
a) the first binding region comprises a heavy chain variable region (VH) comprising the amino acid sequence set forth in SEQ ID NO: 7 or 9 and a light chain variable region (VL) region comprising the amino acid sequence set forth in SEQ ID NO: 8 or 10;
and
b) the second binding region comprises a heavy chain variable region (VH) comprising the amino acid sequence set forth in SEQ ID NO: 17 or 19 and a light chain variable region (VL) region comprising the amino acid sequence set forth in SEQ ID NO: 18 or 20.
9. The binding agent for use of any one of the preceding items, wherein
a) the first binding region comprises a heavy chain variable region (VH) comprising the amino acid sequence set forth in SEQ ID NO: 9 and a light chain variable region (VL) region comprising the amino acid sequence set forth in SEQ ID NO: 10;
and
b) the second binding region comprises a heavy chain variable region (VH) comprising the amino acid sequence set forth in SEQ ID NO: 19 and a light chain variable region (VL) region comprising the amino acid sequence set forth in SEQ ID NO: 20.
10. The binding agent for use of any one of the preceding items, wherein the binding agent is a multispecific antibody, such as a bispecific antibody.
11. The binding agent for use of any one of the preceding items, wherein the binding agent is in the format of a full-length antibody or an antibody fragment.
12. The binding agent for use of any one of items 5-11, wherein each variable region comprises three complementarity determining regions (CDR1, CDR2, and CDR3) and four framework regions (FR1, FR2, FR3, and FR4).
13. The binding agent for use of item 12, wherein said complementarity determining regions and said framework regions are arranged from amino-terminus to carboxy-terminus in the following order: FR1, CDR1, FR2, CDR2, FR3, CDR3, FR4.
14. The binding agent for use of any one of items 5-13, which comprises
i) a polypeptide comprising, consisting of or consisting essentially of, said first heavy chain variable region (VH) and a first heavy chain constant region (CH), and
ii) a polypeptide comprising, consisting of or consisting essentially of, said second heavy chain variable region (VH) and a second heavy chain constant region (CH).
15. The binding agent for use of any one of items 5-14, which comprises
i) a polypeptide comprising said first light chain variable region (VL) and further comprising a first light chain constant region (CL), and
ii) a polypeptide comprising said second light chain variable region (VL) and further comprising a second light chain constant region (CL).
16. The binding agent for use of any one of items 5-15, wherein the binding agent is an antibody comprising a first binding arm and a second binding arm, wherein the first binding arm comprises
i) a polypeptide comprising said first heavy chain variable region (VH) and a first heavy chain constant region (CH), and
ii) a polypeptide comprising said first light chain variable region (VL) and a first light chain constant region (CL);
and the second binding arm comprises
iii) a polypeptide comprising said second heavy chain variable region (VH) and a second heavy chain constant region (CH), and
iv) a polypeptide comprising said second light chain variable region (VL) and a second light chain constant region (CL).
17. The binding agent for use of any one of the preceding items, which comprises
i) a first heavy chain and light chain comprising said antigen-binding region capable of binding to CD40, and
ii) a second heavy chain and light chain comprising said antigen-binding region capable of binding CD137.
18. The binding agent for use of any one of the preceding items, wherein said binding agent comprises
i) a first heavy chain and light chain comprising said antigen-binding region capable of binding to CD40, the first heavy chain comprising a first heavy chain constant region and the first light chain comprising a first light chain constant region; and
ii) a second heavy chain and light chain comprising said antigen-binding region capable of binding CD137, the second heavy chain comprising a second heavy chain constant region and the second light chain comprising a second light chain constant region.
19. The binding agent for use of any one of items 14-18, wherein each of the first and second heavy chain constant regions (CH) comprises one or more of a constant heavy chain 1 (CH1) region, a hinge region, a constant heavy chain 2 (CH2) region and a constant heavy chain 3 (CH3) region, preferably at least a hinge region, a CH2 region and a CH3 region.
20. The binding agent for use of any one of items 14-19, wherein each of the first and second heavy chain constant regions (CHs) comprises a CH3 region and wherein the two CH3 regions comprise asymmetrical mutations.
21. The binding agent for use of any one of items 14-20, wherein in said first heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and in said second heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and wherein said first and said second heavy chains are not substituted in the same positions.
22. The binding agent for use of item 21, wherein (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.
23. The binding agent for use of any of the preceding items, wherein said binding agent induces Fc-mediated effector function to a lesser extent compared to another antibody comprising the same first and second antigen binding regions and two heavy chain constant regions (CHs) comprising human IgG1 hinge, CH2 and CH3 regions.
24. The binding agent for use of item 23, wherein said first and second heavy chain constant regions (CHs) are modified so that the antibody induces Fc-mediated effector function to a lesser extent compared to an antibody which is identical except for comprising non-modified first and second heavy chain constant regions (CHs).
25. The binding agent for use of item 24, wherein each of said non-modified first and second heavy chain constant regions (CHs) comprises the amino acid sequence set forth in SEQ ID NO: 21 or 29.
26. The binding agent for use of item 24 or 25, wherein said Fc-mediated effector function is measured by binding to Fcγ receptors, binding to C1q, or induction of Fe-mediated crosslinking of Fcγ receptors.
27. The binding agent for use of item 26, wherein said Fc-mediated effector function is measured by binding to C1q.
28. The binding agent for use of any one of items 23-27, wherein said first and second heavy chain constant regions have been modified so that binding of C1q to said antibody is reduced compared to a wild-type antibody, preferably reduced by at least 70%, at least 80%, at least 90%, at least 95%, at least 97%, or 100%, wherein C1q binding is preferably determined by ELISA.
29. The binding agent for use of any one of items 14-28, wherein in at least one of said first and second heavy chain constant regions (CH), one or more amino acids in the positions corresponding to positions L234, L235, D265, N297, and P331 in a human IgG1 heavy chain according to EU numbering, are not L, L, D, N, and P, respectively.
30. The binding agent for use of item 29, wherein the positions corresponding to positions L234 and L235 in a human IgG1 heavy chain according to EU numbering are F and E, respectively, in said first and second heavy chains.
31. The binding agent for use of item 29 or 30, wherein the positions corresponding to positions L234, L235, and D265 in a human IgG1 heavy chain according to EU numbering are F, E, and A, respectively, in said first and second heavy chain constant regions (HCs).
32. The binding agent for use of any one of items 29-31, wherein the positions corresponding to positions L234 and L235 in a human IgG1 heavy chain according to EU numbering of both the first and second heavy chain constant regions are F and E, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain is R, or (ii) the position corresponding to K409 in a human IgG1 heavy chain according to EU numbering of the first heavy chain constant region is R, and the position corresponding to F405 in a human IgG1 heavy chain according to EU numbering of the second heavy chain is L.
33. The binding agent for use of any one of items 29-32, wherein the positions corresponding to positions L234, L235, and D265 in a human IgG1 heavy chain according to EU numbering of both the first and second heavy chain constant regions are F, E, and A, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain constant region is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.
34. The binding agent for use of any one of items 14-33, wherein the constant region of said first and/or second heavy chain comprises or consists essentially of or consists of an amino acid sequence selected from the group consisting of
a) the sequence set forth in SEQ ID NO: 21 or 29 [IgG1-FC];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4, at most 3, at most 2 substitutions or at most 1 substitution, compared to the amino acid sequence defined in a) or b).
35. The binding agent for use of any one of items 14-33, wherein the constant region of said first or second heavy chain, such as the second heavy chain, comprises or consists essentially of or consists of an amino acid sequence selected from the group consisting of
a) the sequence set forth in SEQ ID NO: 22 or 30 [IgG1-F405L];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 9 substitutions, such as at most 8, at most 7, at most 6, at most 5, at most 4, at most 3, at most 2 substitutions or at most 1 substitution, compared to the amino acid sequence defined in a) or b).
36. The binding agent for use of any one of items 14-33, wherein the constant region of said first or second heavy chain, such as the first heavy chain comprises or consists essentially of or consists of an amino acid sequence selected from the group consisting of
a) the sequence set forth in SEQ ID NO: 23 or 31 [IgG1-F409R];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).
37. The binding agent for use of any one of items 14-33, wherein the constant region of said first and/or second heavy chain comprises or consists essentially of or consists of an amino acid sequence selected from the group consisting of
a) the sequence set forth in SEQ ID NO: 24 or 32 [IgG1-Fc_FEA];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 7 substitutions, such as at most 6 substitutions, at most 5, at most 4, at most 3, at most 2 substitutions or at most 1 substitution, compared to the amino acid sequence defined in a) or b).
38. The binding agent for use of any one of items 14-37, wherein the constant region of said first and/or second heavy chain, such as the second heavy chain, comprises or consists essentially of or consists of an amino acid sequence selected from the group consisting of
a) the sequence set forth in SEQ ID NO: 25 or 33[IgG1-Fc_FEAL];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 6 substitutions, such as at most 5 substitutions, at most 4 substitutions, at most 3, at most 2 substitutions or at most 1 substitution, compared to the amino acid sequence defined in a) or b).
39. The binding agent for use of any one of items 14-38, wherein the constant region of said first and/or second heavy chain, such as the first heavy chain, comprises or consists essentially of or consists of an amino acid sequence selected from the group consisting of
a) the sequence set forth in SEQ ID NO: 26 or 34 [IgG1-Fc_FEAR];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 6 substitutions, such as at most 5 substitutions, at most 4, at most 3, at most 2 substitutions or at most 1 substitution, compared to the amino acid sequence defined in a) or b).
40. The binding agent for use of any one of the preceding items, wherein said binding agent comprises a kappa (κ) light chain constant region.
41. The binding agent for use of any one of the preceding items, wherein said binding agent comprises a lambda (λ) light chain constant region.
42. The binding agent for use of any one of the preceding items, wherein said first light chain constant region is a kappa (κ) light chain constant region or a lambda (λ) light chain constant region.
43. The binding agent for use of any one of the preceding items, wherein said second light chain constant region is a lambda (λ) light chain constant region or a kappa (κ) light chain constant region.
44. The binding agent for use of any one of the preceding items, wherein said first light chain constant region is a kappa (κ) light chain constant region and said second light chain constant region is a lambda (λ) light chain constant region or said first light chain constant region is a lambda (λ) light chain constant region and said second light chain constant region is a kappa (κ) light chain constant region.
45. The binding agent for use of any one of items 40-44, wherein the kappa (κ) light chain comprises an amino acid sequence selected from the group consisting of
a) the sequence set forth in SEQ ID NO: 27,
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).
46. The binding agent for use of any one of items 41-45, wherein the lambda (λ) light chain comprises an amino acid sequence selected from the group consisting of
a) the sequence set forth in SEQ ID NO: 28,
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).
47. The binding agent for use of any one of the preceding items, wherein the binding agent is of an isotype selected from the group consisting of IgG1, IgG2, IgG3, and IgG4.
48. The binding agent for use of any one of the preceding items, wherein the binding agent is a full-length IgG1 antibody.
49. The binding agent for use of any one of the preceding items, wherein the binding agent is an antibody of the IgG1m(f) allotype.
50. The binding agent for use of any one of the preceding items, wherein the subject is a human subject.
51. The binding agent for use of any one of the preceding items, wherein the tumor or cancer is a solid tumor.
52. The binding agent for use of any one of the preceding items, wherein the tumor or cancer is selected from the group consisting of melanoma, ovarian cancer, lung cancer (e.g., non-small cell lung cancer (NSCLC)), colorectal cancer, head and neck cancer, gastric cancer, breast cancer, renal cancer, urothelial cancer, bladder cancer, esophageal cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, ovarian cancer, endometrial cancer, prostate cancer, penile cancer, cervical cancer, Hodgkin's lymphoma, non-Hodgkin's lymphoma, Merkel cell carcinoma and mesothelioma.
53. The binding agent for use of any one of the preceding items, wherein the tumor or cancer is selected from the group consisting of lung cancer (e.g., non-small cell lung cancer (NSCLC)), melanoma, colorectal cancer, urothelial cancer (cancer of the bladder, ureter, urethra, or renal pelvis), endometrial cancer (EC), breast cancer (e.g., triple negative breast cancer (TNBC)), squamous cell carcinoma of the head and neck (SCCHN) (e.g., cancer of the oral cavity, pharynx or larynx) and cervical cancer.
54. The binding agent for use of any one of the preceding items, wherein the tumor or cancer is selected from the group consisting of lung cancer, melanoma, and colorectal cancer.
55. The binding agent for use of item 54, wherein the cancer is a non-small cell lung cancer (NSCLC), such as a squamous or non-squamous NSCLC.
56. The binding agent for use of item 55, wherein the NSCLC does not have an epidermal growth factor (EGFR)-sensitizing mutation and/or anaplastic lymphoma (ALK) translocation/ROS1 rearrangement.
57. The binding agent for use of item 55 or 56, wherein the subject has received up to four prior systemic treatment regimens for advanced/metastatic disease and has experienced disease progression on or after last prior systemic treatment, such as disease progression determined by radiography.
58. The binding agent for use of item 57, wherein the subject has received platinum-based chemotherapy.
59. The binding agent for use of item 57, wherein the subject is not eligible for platinum-based therapy and has alternative chemotherapy, e.g., a treatment with gemcitabine-containing regimen.
60. The binding agent for use of any one of items 55-59, wherein the subject has received prior treatment with checkpoint inhibitor(s), such as agent(s) targeting PD-1/PD-L, such as a PD-1/PD-L1 inhibitor.
61. The binding agent for use of any one of item 55-60, wherein the subject has experienced disease progression on or after last prior systemic treatment, such as disease progression determined by radiography.
62. The binding agent for use of item 54, wherein the cancer is cutaneous, acral, or mucosal melanoma.
63. The binding agent for use of item 62, wherein the subject has received up to four prior systemic treatments for advanced/metastatic disease and has experienced disease progression on or after last prior systemic treatment, such as disease progression determined by radiography.
64. The binding agent for use of item 62 or 63, wherein the subject has received prior treatment with checkpoint inhibitor(s), such as agent(s) targeting PD-1/PD-L, such as a PD-1/PD-L1 inhibitor.
65. The binding agent for use of item 54, wherein the cancer is cancer is colorectal cancer.
66. The binding agent for use of item 65, wherein the subject has received up to four prior systemic treatments for advanced/metastatic disease and has experienced disease progression on or after last prior systemic treatment, such as disease progression determined by radiography.
67. The binding agent for use of item 65 or 66, wherein the subject has received 5-FU-based therapy.
68. The binding agent for use of any one of items 65-67, wherein the subject has not received treatment with an ICP inhibitor.
69. The binding agent for use of any one of the preceding items, wherein the binding agent is administered in at least one treatment cycle, each treatment cycle being three weeks (21 days).
70. The binding agent for use of any one of the preceding items, wherein one dose is administered every third week (1Q3W).
71. The binding agent for use of any one of the preceding items, wherein one dose is administered on day 1 of each treatment cycle.
72. The binding agent for use of any one of the preceding items, wherein each dose is infused over a minimum of 30 minutes, such as over a minimum of 60 minutes, a minimum of 90 minutes, a minimum of 120 minutes or a minimum of 240 minutes.
73. A composition comprising a binding agent comprising a first binding region binding to human CD40 and a second binding region binding to human CD137, wherein the amount of binding agent in the composition is between about 3-200 mg or about 20×10⁻⁹-1350×10⁻⁹ mol.
74. The composition according to item 73, comprising about 40 mg of said binding agent.
75. The composition according to item 73 or 74, wherein the binding agent is as defined in any one of items 1-72.
76. The composition according to any one of items 73-75, wherein the composition is for systemic administration.
77. The composition according to any one of items 73-76, wherein the composition is for injection or infusion, such as intravenous injection or infusion.
78. The composition according to any one of items 73-77, wherein the binding agent is in aqueous solution, such in 0.9% NaCl (saline), at a volume of 50-500 ml, such as 100-250 ml.
79. The composition according to any one of items 73-78, said composition being a dosage unit form.
80. The composition according to any one of items 73-79 for use in a method for reducing or preventing progression of a tumor or treating cancer in a subject.

Further aspects of the present disclosure are disclosed herein.

### EXAMPLES

### Example 1: Generation of Bispecific Antibodies

The bispecific anti-CD40 anti-4-1BB (herein after referred to as GEN1042 or DuoBody-CD40×4-1BB) was produced with the humanized VH and VL sequences, the human kappa light chain, and a human IgG1 heavy chain described in Table 1. The CD40 binding arm has been produced with the human IgG1 heavy chain containing the following amino acid mutations: L234F, L235E, D265A and F405L (FEAL), wherein the amino acid position number is according to EU numbering (corresponding to SEQ ID NO: 33). The CD137 binding arm has been produced with a human IgG1 heavy chain containing the following amino acid mutations: L234F, L235E, D265A and K409R (FEAR), wherein the amino acid position number is according to EU numbering (correspond to SEQ ID NO: 34).

Bispecific IgG1 antibodies were generated by Fab-arm-exchange under controlled reducing conditions. The basis for this method is the use of complementary CH3 domains, which promote the formation of heterodimers under specific assay conditions as described in WO2011/131746. The F405L and K409R EU numbering) mutations were introduced into the relevant antibodies to create antibody pairs with complementary CH3 domains.

To generate bispecific antibodies, the two parental complementary antibodies, each antibody at a final concentration of 0.5 mg/ml, were incubated with 75 mM 2-mercaptoethylamine-HCl (2-MEA) in 5 a total volume of 100 μL PBS at 31° C. for 5 hours. The reduction reaction was stopped by removing the reducing agent 2-MEA using spin columns (Microcon centrifugal filters, 30 k, Millipore) according to the manufacturer's protocol.

### Example 2: Clinical Trial Design and Preliminary Data

### Trial Design

Clinical trial on GCT1042-01 (ClinicalTrials.gov Identifier: NCT04083599) was designed as a two-part trial, including a dose escalation part and a planned expansion part. The trial was designed as an open-label, multi-center, Phase I/IIa safety trial of GEN1042 ((DuoBody-CD40×4-1BB)). The trial consists of 2 parts; a First-in-Human (FIH) dose escalation (Phase I) and an expansion (Phase IIa).

### Dose Escalation

The dose escalation evaluates GEN1042 in subjects with non-central nervous system (CNS) solid malignant tumors to determine the maximum tolerated dose (MTD) or maximum administered dose (MAD) and/or recommended Phase 2 dose (RP2D). In the dose escalation and/or expansion, subjects receive one infusion of GEN1042 every 3 weeks (q3w) for 21-day cycles until the protocol-defined discontinuation criteria are met (Radiographic disease progression or clinical progression, death, unacceptable adverse events (AEs), investigator decision in the best interest of the patient, withdrawal of consent, pregnancy). The dose escalation evaluates GEN1042 administered intravenously at 10 dose levels at 0.1, 0.3, 1.0, 3.0, 10, 30, 100, 200, 400, and one intermediate dose level at 60 mg fixed. For 0.1, 0.3, 1.0 mg fixed doses. FIG. 2 shows a schematic representation of the dose escalation. The following table enlists the initial dose administered to the individual subjects:

TABLE 5
First Dose by Cancer Type and Subject Number

| Subject | Date of First Exposure | GEN1042 | | |
|---|---|---|---|---|
| Number | to GEN1042 | Dose Level | | Cancer Type |
| 1001 | 17 Sep. 2019 | 0.1 | mg | Other Cancer: Advanced Colon Cancer |
| 1002 | 14 Nov. 2019 | 0.3 | mg | Colorectal Cancer |
| 1003 | 16 Dec. 2019 | 1.0 | mg | NSCLC |
| 1004 | 18 Dec. 2019 | 1.0 | mg | Other Cancer: Metastatic Pancreatic Cancer |
| 1005 | 20 Jan. 2020 | 3.0 | mg | Colorectal Cancer |
| 1006 | 17 Feb. 2020 | 10 | mg | Other Cancer: Adenoid Cystic Carcinoma of Maxilla |
| 1007 | 19 Feb. 2020 | 10 | mg | Other Cancer: Cholangiocarcinoma |
| 1008 | 4 Mar. 2020 | 10 | mg | Melanoma |
| 1009 | 18 Mar. 2020 | 3.0 | mg | Melanoma |
| 1010 | 9 Apr. 2020 | 30 | mg | Other Cancer: Metastatic Ovarian Adenocarcinoma |
| 1011 | 27 Apr. 2020 | 30 | mg | Colorectal Cancer |
| 1012 | 4 May 2020 | 30 | mg | Other Cancer: Urothelial Carcinoma |
| 1013 | 4 May 2020 | 10 | mg | Other Cancer: Neuroendocrine Carcinoma |
| 1014 | 15 May 2020 | 10 | mg | Melanoma |
| 1016 | 19 May 2020 | 10 | mg | Other Cancer: Neuroendocrine Carcinoma |
| 1017 | 8 Jun. 2020 | 100 | mg | Colorectal Cancer |
| 1018 | 9 Jun. 2020 | 30 | mg | Other Cancer: Pleural Mesothelioma |
| 1019 | 16 Jun. 2020 | 100 | mg | Colorectal Cancer |
| 1020^{a} | 22 Jun. 2020 | 100 | mg | |
| 1021 | 29 Jun. 2020 | 30 | mg | NSCLC |
| 1022 | 1 Jul. 2020 | 30 | mg | Other Cancer: Breast Cancer |
| 1023 | 27 Jul. 2020 | 200 | mg | Other Cancer: Oropharyngeal |
| 1024 | 29 Jul. 2020 | 100 | mg | Other Cancer: Metastatic High Grade Myxoid Liposarcoma |
| 1025 | 3 Aug. 2020 | 100 | mg | Melanoma |
| 1026 | 10 Aug. 2020 | 200 | mg | Colorectal Cancer |
| 027 | 19 Aug. 2020 | 100 | mg | Colorectal Cancer |
| 1028 | 19 Aug. 2020 | 200 | mg | Other Cancer: Mesothelioma |
| 1029 | 26 Aug. 2020 | 3.0 | mg | Other Cancer: Adrenal Cortical Carcinoma |
| 1030 | 26 Aug. 2020 | 3.0 | mg | Colorectal Cancer |
| 1031 | 30 Sep. 2020 | 200 | mg | Melanoma |
| 1032 | 14 Oct. 2020 | 200 | mg | Melanoma |
| 1033 | 7 Oct. 2020 | 200 | mg | Other Cancer: Adenocarcinoma of Cervix |
| 1034 | 29 Oct. 2020 | 30 | mg | NSCLC |
| 1035 | 4 Nov. 2020 | 30 | mg | Other Cancer: Triple Negative Breast Cancer |
| 1036 | 12 Nov. 2020 | 30 | mg | Other Cancer: High Grade Glioma; Malignant Carcinoid Tumor |
| | | | | of Lung |
| 1037 | 1 Dec. 2020 | 400 | mg | Other Cancer: Adenoid Cystic Carcinoma |
| 1038 | 3 Dec. 2020 | 400 | mg | NSCLC |
| 1603 | 15 Dec. 2020 | 400 | mg | Other Cancer: Primary Malignant Neoplasm of Hypopharynx |
| 1604 | 17 Dec. 2020 | 60 | mg | Other Cancer: HNSCC |

### Inclusion Criteria

For dose escalation, subject is required to be a man or woman ˜18 years of age and are required to have measurable disease according to RECIST 1.1. Subjects are required to have a histologically or cytologically confirmed non-CNS solid tumor that are metastatic or unresectable and for whom there is no available standard therapy likely to confer clinical benefit, or subjects who are not candidates for such available therapy, and for whom, in the opinion of the investigator, experimental therapy with GEN1042 could be beneficial. Subject must have Eastern Cooperative Oncology Group (ECOG) Performance Status 0-1. Subject must have acceptable bone marrow and hematological, liver and renal functions

### Results

### Trial Continuation

As of the data extraction date of 8 Jan. 2021, preliminary data for the 39 subjects treated with GEN1042 showed that 10 subjects across all 10 dose levels (0.1 mg to 400 mg) were continuing to receive trial drug (Table 6). Of the 29 subjects who discontinued treatment with GEN1042, 15 subjects discontinued trial treatment due to documented radiographic progressive disease, 6 subjects discontinued due to clinical progression, 2 subjects discontinued due to subject request to discontinue trial treatment, 3 subjects discontinued due to adverse event(s) (AE[s]). Three subjects discontinued due to other reasons:
Subject 1001 (0.1 mg): The investigator was concerned that the subject was not receiving therapeutic benefit due to the low dose; 2 disease evaluations showed a trend toward disease progression.
Subject 1006 (10 mg): Consideration of benefit/risk of trial participation during the COVID 19 pandemic.
Subject 1014 (10 mg): Investigator decision.

Three of the 15 subjects who came off treatment due to documented radiographic disease progression later died during the trial follow-up period.

TABLE 6
Discontinuation from Treatment - All Subjects

| | GEN1042 Doses | | | | | | | | | | |
|---|---|---|---|---|---|---|---|---|---|---|---|
| | ≤1 mg | | 3.0 mg | 10 mg | 30 mg | 60 mg | 100 mg | 200 mg | 400 mg | Total | |
| | (N = 4) | | (N = 4) | (N = 6) | (N = 9) | (N = 1) | (N = 6) | (N = 6) | (N = 3) | (N = 39) | |
| | n (%) | | n (%) | n (%) | n (%) | n (%) | n (%) | n (%) | n (%) | n (%) | |
| Subjects ongoing treatment | 0 | | 1 (25.0) | 1 (16.7) | 1 (11.1) | 1 (100) | 1 (16.7) | 2 (33.3) | 3 (100) | 10 | (25.6) |
| Subjects who discontinued | 4 | (100) | 3 (75.0) | 5 (83.3) | 8 (88.9) | 0 | 5 (83.3) | 4 (66.7) | 0 | 29 | (74.4) |
| Treatment | | | | | | | | | | | |
| Documented radiographic | 1 | (25.0) | 3 (75.0) | 1 (16.7) | 4 (44.4) | | 4 (66.7) | 2 (33.3) | | 15 | (38.5) |
| disease progression | | | | | | | | | | | |
| Clinical Progression | 2 | (50.0) | | 2 (33.3) | 2 (22.2) | | | | | 6 | (15.4) |
| Subject request to | | | | | 1 (11.1) | | | 1 (16.7) | | 2 | (5.1) |
| discontinue trial treatment | | | | | | | | | | | |
| Death | | | | | | | | | | 0 | |
| Poor/non-compliance | | | | | | | | | | 0 | |
| Pregnancy | | | | | | | | | | 0 | |
| Adverse Event(s) | | | | | 1 (11.1) | | 1 (16.7) | 1 (16.7) | | 3 | (7.7) |
| Sponsor Decision | | | | | | | | | | 0 | |
| Lost to Follow-Up | | | | | | | | | | 0 | |
| Other | 1 | (25.0) | 0 | 2 (33.3) | 0 | | | | | 3 | (7.7) |

Subjects who discontinued treatment continued on the trial in the follow-up period. As of the 8 Jan. 2021 data cut-off date, 25 subjects were continuing in the trial (Table 7). Of the 14 subjects who discontinued from trial GCT1042-01,1 subject withdrew consent from trial, 10 died, and 2 discontinued from the trial due to other reasons:
Subject 1004 (1 mg): progressive disease.
Subject 1025 (100 mg): not suitable for 30 day and ongoing safety follow up visit/phone calls due to rapid clinical progression.

TABLE 7
Discontinuation from Trial - All Subjects

| | GEN1042 Doses | | | | | | | | | |
|---|---|---|---|---|---|---|---|---|---|---|
| | ≤1 mg | | 3.0 mg | 10 mg | 30 mg | 60 mg | 100 mg | 200 mg | 400 mg | Total |
| | (N = 4) | | (N = 4) | (N = 6) | (N = 9) | (N = 1) | (N = 6) | (N = 6) | (N = 3) | (N = 39) |
| | n (%) | | n (%) | n (%) | n (%) | n (%) | n (%) | n (%) | n (%) | n (%) |
| Subjects ongoing in trial | 0 | | 4 (100) | 5 (83.3) | 3 (33.3) | 1 (100) | 5 (83.3) | 4 (66.7) | 3 (100) | 25 (64.1) |
| Subjects no longer ongoing | 4 | (100) | 0 | 1 (16.7) | 6 (66.7) | 0 | 1 (16.7) | 2 (33.3) | 0 | 14 (35.9) |
| Subject withdrew consent | | | | | | | | 1 (16.7) | | 1 (2.6) |
| from trial | | | | | | | | | | |
| Death | 3 | (75.0) | | 1 (16.7) | 6 (66.7) | | | 1 (16.7) | | 11 (28.2) |
| Sponsor Decision | | | | | | | | | | 0 |
| Lost to Follow-up | | | | | | | | | | 0 |
| Maximal per protocol trial | | | | | | | | | | 0 |
| duration of 3 years after | | | | | | | | | | |
| first infusion reached | | | | | | | | | | |
| Other | 1 | (25.0) | | | | | 1 (16.7) | | | 2 (5.1) |

### Preliminary Efficacy

As of the data cut-off date of 8 Jan. 2021, preliminary efficacy data from 39 subjects who had exhausted standard of care therapy known to confer clinical benefit were treated in the dose escalation phase of the FIH clinical trial. GEN1042 doses ranging from 0.1 to 400 mg were given once every 3 weeks (1Q3W). Of the 39 subjects enrolled in the study, one subject had a confirmed PR and was ongoing in the trial at the time of data cut-off and disease stabilization was achieved in 20 subjects. The confirmed objective response rate is 1 (2.6%) and the confirmed disease control rate is 20 (51.3%) (Table 8).

TABLE 8
Confirmed Objective Response Rate - Summary Table - All Subjects

| | GEN1042 Doses | | | | | | | | | |
|---|---|---|---|---|---|---|---|---|---|---|
| | ≤1 mg | 3.0 mg | 10 mg | 30 mg | 60 mg | 100 mg | 200 mg | 400 mg | Total | |
| | N (%) | N (%) | N (%) | N (%) | N (%) | N (%) | N (%) | N (%) | N (%) | |
| N | 4 | 4 | 6 | 9 | 1 | 6 | 6 | 3 | 39 | |
| Confirmed Best | | | | | | | | | | |
| Overall Response | | | | | | | | | | |
| CR (Complete | | | | | | | | | 0 | |
| Response) | | | | | | | | | | |
| PR (Partial | | 1 (25.0) | | | | | | | 1 | (2.6) |
| Response) | | | | | | | | | | |
| SD (Stable Disease) | 3 (75.0) | 2 (50.0) | 5 (83.3) | 4 (44.4) | | 2 (33.3) | 3 (50.0) | | 19 | (48.7) |
| PD (Progressive | 1 (25.0) | 1 (25.0) | 1 (16.7) | 3 (33.3) | | 3 (50.0) | 2 (33.3) | | 11 | (28.2) |
| Disease) | | | | | | | | | | |
| NE (Not Evaluable) | | | | | | 1 (16.7) | 1 (16.7) | | 2 | (5.1) |
| NA (Not Applicable) | | | | 2 (22.2) | 1 (100) | | | 3 (100) | 6 | (15.4) |
| Confirmed Objective | | 1 (25.0) | | | | | | | 1 | (2.6) |
| Response (CR + PR) | | | | | | | | | | |
| Rate | | | | | | | | | | |
| Confirmed Disease | 3 (75.0) | 3 (75.0) | 5 (83.3) | 4 (44.4) | | 2 (33.3) | 3 (50.0) | | 20 | (51.3) |
| Control (CR + PR + SD) | | | | | | | | | | |
| Rate | | | | | | | | | | |

Percentage change in tumour measurements over time in 39 evaluable patients enrolled in the Phase 1 dose escalation of the GCT1042-01 trial who at least one post-baseline lesion assessment in FIG. 3. Although Subject 1022 (30 mg)—mTNBC experienced a −24.4% reduction (SD) in tumor burden at week 6 and had a new surgery with one of the TLs resected before the PR response, so the observed PR may not be a true PR.

### Adverse Events

Treatment emergent adverse events (TEAEs) occurred in most subjects. The most common TEAEs (occurring in ≥10% of subjects) were fatigue, nausea, decreased appetite, aspartate aminotransferase (AST) increase, diarrhea, alanine aminotransferase (ALT) increase, pyrexia, anemia, arthralgia, constipation, headache, pruritus, dyspnea, vomiting, dizziness, flushing, and urinary tract infection. The majority of the TEAEs were grade 1 or grade 2.

Serious adverse events (SAEs) were reported in 15 (38.5%) subjects (Table 9). The most common SAE reported (3 subjects, 7.7%) was ALT increased.

Three subjects experienced serious TEAEs that were considered related to GEN1042 (Table 10):
One subject (Subject 1024, 100 mg dose group) experienced grade 3 ALT increased, grade 3 aspartate aminotransferase (AST) increase, grade 1 blood bilirubin increased, and grade 4 neutropenia. These events were considered serious.
One subject (Subject 1023, 200 mg dose group) experienced grade 4 alanine aminotransferase (ALT) increase/grade 4 AST increased. These events were considered serious. The grade 4 ALT/aspartate aminotransferase (AST) increase met DLT criteria.
One subject (Subject 1033, 200 mg dose group) experienced grade 3 alanine aminotransferase (ALT) increase and grade 1 pyrexia. These events were considered serious.

Grading has been assigned in accordance with the National Cancer Institute's Common Terminology Criteria for Adverse Events (CTCAE) version 5.0.

The 3 subjects above experienced serious alanine aminotransferase (ALT) increase and/or aspartate aminotransferase (AST) elevations, but none of them met the criteria for a drug-induced liver injury (DILI). At the time of the 8 Jan. 2021 data cut-off, no treatment-related deaths had occurred.

Clinical activity was observed at the 3 mg, 30 mg, and the 200 mg dose levels 1Q3W; however, safety data indicate that the 200 mg 1Q3W dose level is associated with a higher incidence of treatment-related SAEs and grade ≥3 AEs, which primarily consisted of AST/ALT elevation that resolved with treatment. Taken together, the data indicates that the a preferred 1Q3W dose level would be about 100 mg.

TABLE 9
Most Common SAEs

| | <=1 mg | | 3.0 mg | | 10 mg | | 30 mg | | 60 mg | | 100 mg | | 200 mg | | 400 mg | | Total | |
|---|---|---|---|---|---|---|---|---|---|---|---|---|---|---|---|---|---|---|
| | (N = 4) | | (N = 4) | | (N = 6) | | (N = 9) | | (N = 1) | | (N = 6) | | (N = 6) | | (N = 3) | | (N = 39) | |
| | n | (%) | n | (%) | n | (%) | n | (%) | n | (%) | n | (%) | n | (%) | n | (%) | n | (%) |
| Patients with at least | 2 | (50.0) | 0 | | 2 | (33.3) | 4 | (44.4) | 0 | | 4 | (66.7) | 2 | (33.3) | 1 | (33.3) | 15 | (38.5) |
| one AE | | | | | | | | | | | | | | | | | | |
| Alanine aminotransferase | 0 | | 0 | | 0 | | 0 | | 0 | | 1 | (16.7) | 2 | (33.3) | 0 | | 3 | (7.7) |
| increased | | | | | | | | | | | | | | | | | | |
| Aspartate aminotrans- | 0 | | 0 | | 0 | | 0 | | 0 | | 1 | (16.7) | 1 | (16.7) | 0 | | 2 | (5.1) |
| ferase increased | | | | | | | | | | | | | | | | | | |
| Dehydration | 0 | | 0 | | 1 | (16.7) | 1 | (11.1) | 0 | | 0 | | 0 | | 0 | | 2 | (5.1) |
| Dyspnoea | 1 | (25.0) | 0 | | 0 | | 1 | (11.1) | 0 | | 0 | | 0 | | 0 | | 2 | (5.1) |
| Pneumonia | 0 | | 0 | | 0 | | 1 | (11.1) | 0 | | 1 | (16.7) | 0 | | 0 | | 2 | (5.1) |
| Pulmonary embolism | 1 | (25.0) | 0 | | 0 | | 0 | | 0 | | 0 | | 0 | | 1 | (33.3) | 2 | (5.1) |
| Pyrexia | 0 | | 0 | | 1 | (16.7) | 0 | | 0 | | 0 | | 1 | (16.7) | 0 | | 2 | (5.1) |

TABLE 10
Treatment-Related SAEs

| | <=1 mg | | 3.0 mg | | 10 mg | | 30 mg | | 60 mg | | 100 mg | | 200 mg | | 400 mg | | Total | |
|---|---|---|---|---|---|---|---|---|---|---|---|---|---|---|---|---|---|---|
| | (N = 4) | | (N = 4) | | (N = 6) | | (N = 9) | | (N = 1) | | (N = 6) | | (N = 6) | | (N = 3) | | (N = 39) | |
| | n | (%) | n | (%) | n | (%) | n | (%) | n | (%) | n | (%) | n | (%) | n | (%) | n | (%) |
| Patients with at least one AE | 0 | | 0 | | 0 | | 0 | | 0 | | 1 | (16.7) | 2 | 33.3) | 0 | | 3 | (7.7 |
| Alanine aminotransferase increased | 0 | | 0 | | 0 | | 0 | | 0 | | 1 | (16.7) | 2 | (33.3) | 0 | | 3 | (7.7) |
| Aspartate aminotransferase increased | 0 | | 0 | | 0 | | 0 | | 0 | | 1 | (16.7) | 1 | (16.7) | 0 | | 2 | (5.1) |
| Blood bilirubin increased | 0 | | 0 | | 0 | | 0 | | 0 | | 1 | (16.7) | 0 | | 0 | | 1 | (2.6) |
| Neutropenia | 0 | | 0 | | 0 | | 0 | | 0 | | 1 | (16.7) | 0 | | 0 | | 1 | (2.6) |
| Pyrexia | 0 | | 0 | | 0 | | 0 | | 0 | | 0 | | 1 | (16.7) | 0 | | 1 | (2.6) |

### Example 3: Physiological Based Pharmacokinetics/Pharmacodynamic Modelling

An integrated minimal physiologically based pharmacokinetic/pharmacodynamic (PBPK/PD) model was developed that models the distribution of GEN1042 into plasma, peripheral tissues including liver, tumor and lymph compartment. The model leverages PK and PD data as well as physiological parameters from literature for parameterizations of expressions of CD40 and 4-1BB, and T-cell trafficking into these cells. Model compartments consist of well-mixed 2- and 3-dimensional spaces, with the 2-dimensional, cell membrane space being further divided into membrane within an immunological synapse (IS) between two cells, and membrane outside the IS. Free drug transfers between the fluid space and receptors on a cell surface that are not contained in an immunological synapse. Receptors (bound to GEN1042 or unbound) diffuse into and out of IS space. The model incorporates dynamic binding of GEN1042/receptor complex in IS to either CD40 or 4-1BB to predict trimer (crosslinked GEN1046 to CD40 and 4-1BB) formation.

The model was used to explore the predicted in vivo trimer formation at various dosing regimens. Specifically, simulations were done to predict trimer levels on CD8⁺ and CD4⁺ T-cells in tumor, lymph nodes (LN) and liver compartment, to predict 4-1BB engagement on T-cells and CD40 engagement on APCs. Specifically, macrophages, mature dendritic cells (mDCs), and B-cells were evaluated for CD40 engagement based on their expression patterns in each compartment. To summarize trimer engagement over a single 3-week dosing interval, area under the curve (AUC) for the predicted percent trimer (of total 4-1BB or CD40) at different doses were plotted for 4-1BB engagement on T-cells and CD40 engagement on APCs.

FIGS. 4A, 4B and 4C shows AUC for predicted trimer levels with respect to dose. Maximum engagement for 4-1BB on T-cells was observed in range of 100-200 mg in tumors and LN, and around 50-200 mg in liver. Engagement for CD40 was observed at similar rage on APCs. Increasing doses >200 mg resulted in reduced trimer formation. In addition, based on available clinical pharmacodynamic data, higher magnitude, and consistent modulation of peripheral pharmacodynamic endpoints (IFN-γ and proliferating Ki67+ effector memory CD8+ T cells) were seen at dose levels up to 200 mg. Considering, PBPK/PD modeling predictions and available clinical data, the optimal dose of GEN1042 was predicted to be in the range of 100 mg 1Q3W.

### Example 4: Pharmacodynamic Evaluation of GEN1042 in Peripheral Blood in Patients with Advanced Solid Tumors

To investigate the biological activity of GEN1042 at various dose levels in patients with advanced tumors, blood and serum samples were collected at baseline and at multiple time points on treatment. Based on the mechanism of action of GEN1042, it was anticipated that dose levels with biological activity will modulate circulating levels of interferon-γ (IFN-γ) and IFN-γ-inducible macrophage/dendritic cell soluble factor, thymus and activation-regulated chemokine (TARC), as well as induce proliferation/activation of peripheral CD8 T cells.

To determine serum levels of IFN-γ and TARC, samples were collected from patients at baseline and at multiple time points post administration of GEN1042 in cycle 1 and cycle 2 (days 1 [pre, 2 h and between 4-6 h post-administration], 2, 3, 8, and 15) as well as pre-dose at cycle 3 and beyond. Serum levels of IFN-γ and TARC were measured by a Meso Scale Discovery (MSD) multiplex immune-assay (cat. no. K15209G) following the manufacturer's instructions.

Administration of GEN1042 to cancer patients resulted in modulation of circulating levels of IFN-γ (FIG. 5A) and TARC (FIG. 5B). In the preliminary data set, levels of IFN-γ exceeded the normal reference range (<11.81 pg/mL) in the first two cycles for at least 1 patient per dose ≥3 mg. In this range, peak induction occurred 2-7 days post dosing, with a maximal induction observed at the 100 mg and 200 mg dose levels. Although modulation of IFN-γ is observed at the 400 mg dose level, the mean induction during the first treatment cycle appears lower compared to 100 and 200 mg dose levels. TARC levels consistently exceeded the normal reference range (<513 pg/mL) in the first two cycles for at least 1 patient per dose ≥3 mg. In this range, peak induction occurred 2-7 days post dosing, with a maximal induction observed at the 30 mg and 100 mg dose levels.

To measure peripheral modulation of immune cells subsets, immunophenotyping of peripheral blood was conducted in whole blood collected in EDTA tubes at baseline and at multiple time points post GEN1042 administration in cycle 1 and cycle 2 (days 1, 2, 3, 8 and 15) as well as pre-dose at cycle 3 and beyond. 100 μL of whole blood was added to fluorochrome-conjugated monoclonal antibodies that bind specifically to cell surface antigens: CD45RA-FITC (clone L48, BD Biosciences cat. no. 335039), CCR7-BV510 (clone 3D12, BD Biosciences, cat. no. 563449), CD8-PerCP-Cy5.5 (clone RPA-T8, BD Biosciences, cat. no. 560662), CD4-PE (clone SK3, BD Biosciences, cat. no. 345769), CD45-BV605 (clone H130, BD Biosciences cat. no. 564047), CD19-PE-Cy7 (clone Sj2SC1, BD Biosciences, cat. no. 341113), CD3-APC-H7 (clone SK7, BD Biosciences, cat. no. 560176) and 4-1BB-AF647 (clone 4B4-1, Biolegend, cat. no. 309824). After incubation on ice, the stained samples were treated with FACS Lysing Solution (BD Biosciences, Catalog No 349202) to lyse erythrocytes. Excess antibody and cell debris were removed by washing with Stain Buffer (BD Biosciences, cat. no. 554656). Following lyse/wash, cells were fixed and permeabilized by incubation with Permeabilizing Solution 2 buffer (BD Biosciences, cat. no. 340973). Next, cells were washed and resuspended in Stain Buffer and incubated on ice with antibody to Ki67-BV421 (clone B56, BD Biosciences, cat. no. 562899) to detect proliferating cells. After incubation, excess antibody was removed by washing with Stain Buffer. Cells were resuspended in Stain Buffer and acquired on a BD FACSCanto™ flow cytometer (Becton Dickinson) within 1 hour of staining.

GEN1042 elicited transient trafficking/margination of CD8 T cells (FIG. 6A) and B cells (FIG. 6B) post dosing indicative of 4-1BB and CD40 target engagement, respectively. This was observed consistently at doses ≥3 mg with more pronounced B cell trafficking at doses ≥30 mg. Additionally, evidence of CD4 and CD8 T cell maturation/expansion was observed in patients dosed with GEN1042 (FIG. 7). Area Under the Curve (AUC) values were calculated for each patient using baseline-normalized values out to cycle 2 day 15 for CD4 and CD8 naïve (CD45RA+CCR7+) or effector memory (CD45RA-CCR7-) [Tem] T cells. Then the average AUC was computed for patients within each dose level and the difference between naïve and Tem cells within the CD4 or CD8 populations was calculated. Longitudinal patient profiles displayed consistent shifts from naïve T cells towards Tem cells at doses ≥30 mg.

GEN1042 elicited proliferation and activation of total CD8+ T cells (FIGS. 8A, 9A) and CD8+ effector memory T cells (FIGS. 8B, 9B) as measured by an increase in the frequency of % Ki67+ and %4-1BB+ populations, respectively. Peak immunophenotypic changes occurred approximately 7 days post dosing. Of note, doses ≥200 mg displayed a reduced induction of 4-1BB in cycle 2 relative to 100 mg dosed patients. Comparable to the changes observed with modulation of circulating levels of IFN-γ, more consistent modulation of proliferation and activation was observed in patients dosed at >10 mg (Ki67) and >3 mg (4-1BB).

### CONCLUSION

GEN1042 elicited pharmacodynamics across a broad range of dose levels, characterized by modulation of immune effector cells and soluble factors critical for the generation of antitumor immune responses, that were favorable between 30-200 mg.

### Analysis Notes

Pharmacodynamic assessments, including changes in circulating levels of cytokines, chemokines and immune cell populations, were conducted using blood samples from patients with advanced solid tumors enrolled in the dose escalation phase of an open-label, multi-center safety trial of GEN1042 (NCT04083599). Data cut off for analyses (DCO) was Jan. 22, 2021.

Available safety, efficacy, PK/PD data as well as mechanistic PKPD modeling were used to guide the GEN1042 dose recommended for further evaluation. Translational research analyses showed favorable target engagement, and T- and B-cell activation at dose levels ranging from 30 and 200 mg 1Q3W. Pharmacokinetic/pharmacodynamic peripheral blood and receptor occupancy modeling showed peak trimer formation in tumors and lymph nodes occurring between 100 mg and 200 mg.

1. A binding agent for use in a method for reducing or preventing progression of a tumor or treating cancer in a subject, said method comprising administering to said subject the binding agent in a suitable amount, wherein the binding agent comprises a first binding region binding to human CD40, such as human CD40 comprising the sequence set forth in SEQ ID NO: 36, and a second binding region binding to human CD137, such as human CD137 comprising the sequence set forth in SEQ ID NO: 38.

2. The binding agent for use of claim 1, wherein the suitable amount of the binding agent is a therapeutically effective and safe amount.

3. The binding agent for use of any one of the preceding claims, wherein the suitable amount of the binding agent is about 0.04-2.5 mg/kg body weight or about 3-200 mg in total; and/or about 0.25×10⁻⁹-16.9×10⁻⁹ mol/kg body weight or about 20×10⁻⁹-1350×10⁻⁹ mol in total.

4. The binding agent for use of any one of the preceding claims, wherein the binding agent is administered systemically, preferably intravenously.

5. The binding agent for use of any one of the preceding claims, wherein
a) the first binding region comprises a heavy chain variable region (VH) comprising the CDR1, CDR2, and CDR3 sequences of SEQ ID NO: 7 or 9, and a light chain variable region (VL) comprising the CDR1, CDR2, and CDR3 sequences of SEQ ID NO: 8 or 10;
and
b) the second antigen-binding region comprises a heavy chain variable region (VH) comprising the CDR1, CDR2, and CDR3 sequences of SEQ ID NO: 17 or 19, and a light chain variable region (VL) comprising the CDR1, CDR2, and CDR3 sequences of SEQ ID NO: 18 or 20.

6. The binding agent for use of any one of the preceding claims, wherein
a) the first binding region comprises a heavy chain variable region (VH) comprising the CDR1, CDR2, and CDR3 sequences set forth in: SEQ ID NO: 1, 2, and 3, respectively, and a light chain variable region (VL) comprising the CDR1, CDR2, and CDR3 sequences set forth in: SEQ ID NO: 4, 5, and 6, respectively; and
b) the second antigen-binding region comprises a heavy chain variable region (VH) comprising the CDR1, CDR2, and CDR3 sequences set forth in: SEQ ID NO: 11, 12, and 13, respectively, and a light chain variable region (VL) comprising the CDR1, CDR2, and CDR3 sequences set forth in: SEQ ID NO: 14, 15, and 16, respectively.

7. The binding agent for use of any one of the preceding claims, wherein
a) the first binding region comprises a heavy chain variable region (VH) comprising an amino acid sequence having at least 90%, at least 95%, at least 97%, at least 99%, or 100% sequence identity to SEQ ID NO: 7 or 9 and a light chain variable region (VL) region and comprising an amino acid sequence having at least 90%, at least 95%, at least 97%, at least 99%, or 100% sequence identity to SEQ ID NO: 8 or 10;
b) the second binding region comprises a heavy chain variable region (VH) comprising an amino acid sequence having at least 90%, at least 95%, at least 97%, at least 99%, or 25 100% sequence identity to SEQ ID NO: 17 or 19 and a light chain variable region (VL) region comprising an amino acid sequence having at least 90%, at least 95%, at least 97%, at least 99%, or 100% sequence identity to SEQ ID NO: 18 or 20.

8. The binding agent for use of any one of the preceding claims, wherein
a) the first binding region comprises a heavy chain variable region (VH) comprising the amino acid sequence set forth in SEQ ID NO: 7 or 9 and a light chain variable region (VL) region comprising the amino acid sequence set forth in SEQ ID NO: 8 or 10;
and
b) the second binding region comprises a heavy chain variable region (VH) comprising the amino acid sequence set forth in SEQ ID NO: 17 or 19 and a light chain variable region (VL) region comprising the amino acid sequence set forth in SEQ ID NO: 18 or 20.

9. The binding agent for use of any one of the preceding claims, wherein
a) the first binding region comprises a heavy chain variable region (VH) comprising the amino acid sequence set forth in SEQ ID NO: 9 and a light chain variable region (VL) region comprising the amino acid sequence set forth in SEQ ID NO: 10;
and
b) the second binding region comprises a heavy chain variable region (VH) comprising the amino acid sequence set forth in SEQ ID NO: 19 and a light chain variable region (VL) region comprising the amino acid sequence set forth in SEQ ID NO: 20.

10. The binding agent for use of any one of the preceding claims, wherein the binding agent is a multispecific antibody, such as a bispecific antibody.

11. The binding agent for use of any one of the preceding claims, wherein the binding agent is in the format of a full-length antibody or an antibody fragment.

12. The binding agent for use of any one of claims 5-11, wherein each variable region comprises three complementarity determining regions (CDR1, CDR2, and CDR3) and four framework regions (FR1, FR2, FR3, and FR4).

13. The binding agent for use of claim 12, wherein said complementarity determining regions and said framework regions are arranged from amino-terminus to carboxy-terminus in the following order: FR1, CDR1, FR2, CDR2, FR3, CDR3, FR4.

14. The binding agent for use of any one of claims 5-13, which comprises
i) a polypeptide comprising, consisting of or consisting essentially of, said first heavy chain variable region (VH) and a first heavy chain constant region (CH), and
ii) a polypeptide comprising, consisting of or consisting essentially of, said second heavy chain variable region (VH) and a second heavy chain constant region (CH).

15. The binding agent for use of any one of claims 5-14, which comprises
i) a polypeptide comprising said first light chain variable region (VL) and further comprising a first light chain constant region (CL), and
ii) a polypeptide comprising said second light chain variable region (VL) and further comprising a second light chain constant region (CL).

16. The binding agent for use of any one of claims 5-15, wherein the binding agent is an antibody comprising a first binding arm and a second binding arm, wherein
the first binding arm comprises
i) a polypeptide comprising said first heavy chain variable region (VH) and a first heavy chain constant region (CH), and
ii) a polypeptide comprising said first light chain variable region (VL) and a first light chain constant region (CL);
and the second binding arm comprises
iii) a polypeptide comprising said second heavy chain variable region (VH) and a second heavy chain constant region (CH), and
iv) a polypeptide comprising said second light chain variable region (VL) and a second light chain constant region (CL).

17. The binding agent for use of any one of the preceding claims, which comprises
i) a first heavy chain and light chain comprising said antigen-binding region capable of binding to CD40, and
ii) a second heavy chain and light chain comprising said antigen-binding region capable of binding CD137.

18. The binding agent for use of any one of the preceding claims, wherein said binding agent comprises
i) a first heavy chain and light chain comprising said antigen-binding region capable of binding to CD40, the first heavy chain comprising a first heavy chain constant region and the first light chain comprising a first light chain constant region; and
ii) a second heavy chain and light chain comprising said antigen-binding region capable of binding CD137, the second heavy chain comprising a second heavy chain constant region and the second light chain comprising a second light chain constant region.

19. The binding agent for use of any one of claims 14-18, wherein each of the first and second heavy chain constant regions (CH) comprises one or more of a constant heavy chain 1 (CH1) region, a hinge region, a constant heavy chain 2 (CH2) region and a constant heavy chain 3 (CH3) region, preferably at least a hinge region, a CH2 region and a CH3 region.

20. The binding agent for use of any one of claims 14-19, wherein each of the first and second heavy chain constant regions (CHs) comprises a CH3 region and wherein the two CH3 regions comprise asymmetrical mutations.

21. The binding agent for use of any one of claims 14-20, wherein in said first heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and in said second heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and wherein said first and said second heavy chains are not substituted in the same positions.

22. The binding agent for use of claim 21, wherein (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.

23. The binding agent for use of any of the preceding claims, wherein said binding agent induces Fc-mediated effector function to a lesser extent compared to another antibody comprising the same first and second antigen binding regions and two heavy chain constant regions (CHs) comprising human IgG1 hinge, CH2 and CH3 regions.

24. The binding agent for use of claim 23, wherein said first and second heavy chain constant regions (CHs) are modified so that the antibody induces Fc-mediated effector function to a lesser extent compared to an antibody which is identical except for comprising non-modified first and second heavy chain constant regions (CHs).

25. The binding agent for use of claim 24, wherein each of said non-modified first and second heavy chain constant regions (CHs) comprises the amino acid sequence set forth in SEQ ID NO: 21 or 29.

26. The binding agent for use of claim 24 or 25, wherein said Fc-mediated effector function is measured by binding to Fcγ receptors, binding to C1q, or induction of Fe-mediated crosslinking of Fcγ receptors.

27. The binding agent for use of claim 26, wherein said Fc-mediated effector function is measured by binding to C1q.

28. The binding agent for use of any one of claims 23-27, wherein said first and second heavy chain constant regions have been modified so that binding of C1q to said antibody is reduced compared to a wild-type antibody, preferably reduced by at least 70%, at least 80%, at least 90%, at least 95%, at least 97%, or 100%, wherein C1q binding is preferably determined by ELISA.

29. The binding agent for use of any one of claims 14-28, wherein in at least one of said first and second heavy chain constant regions (CH), one or more amino acids in the positions corresponding to positions L234, L235, D265, N297, and P331 in a human IgG1 heavy chain according to EU numbering, are not L, L, D, N, and P, respectively.

30. The binding agent for use of claim 29, wherein the positions corresponding to positions L234 and L235 in a human IgG1 heavy chain according to EU numbering are F and E, respectively, in said first and second heavy chains.

31. The binding agent for use of claim 29 or 30, wherein the positions corresponding to positions L234, L235, and D265 in a human IgG1 heavy chain according to EU numbering are F, E, and A, respectively, in said first and second heavy chain constant regions (HCs).

32. The binding agent for use of any one of claims 29-31, wherein the positions corresponding to positions L234 and L235 in a human IgG1 heavy chain according to EU numbering of both the first and second heavy chain constant regions are F and E, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain is R, or (ii) the position corresponding to K409 in a human IgG1 heavy chain according to EU numbering of the first heavy chain constant region is R, and the position corresponding to F405 in a human IgG1 heavy chain according to EU numbering of the second heavy chain is L.

33. The binding agent for use of any one of claims 29-32, wherein the positions corresponding to positions L234, L235, and D265 in a human IgG1 heavy chain according to EU numbering of both the first and second heavy chain constant regions are F, E, and A, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain constant region is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.

34. The binding agent for use of any one of claims 14-33, wherein the constant region of said first and/or second heavy chain comprises or consists essentially of or consists of an amino acid sequence selected from the group consisting of
a) the sequence set forth in SEQ ID NO: 21 or 29 [IgG1-FC];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4, at most 3, at most 2 substitutions or at most 1 substitution, compared to the amino acid sequence defined in a) or b).

35. The binding agent for use of any one of claims 14-33, wherein the constant region of said first or second heavy chain, such as the second heavy chain, comprises or consists essentially of or consists of an amino acid sequence selected from the group consisting of
a) the sequence set forth in SEQ ID NO: 22 or 30 [IgG1-F405L];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 9 substitutions, such as at most 8, at most 7, at most 6, at most 5, at most 4, at most 3, at most 2 substitutions or at most 1 substitution, compared to the amino acid sequence defined in a) or b).

36. The binding agent for use of any one of claims 14-33, wherein the constant region of said first or second heavy chain, such as the first heavy chain comprises or consists essentially of or consists of an amino acid sequence selected from the group consisting of
a) the sequence set forth in SEQ ID NO: 23 or 31 [IgG1-F409R];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).

37. The binding agent for use of any one of claims 14-33, wherein the constant region of said first and/or second heavy chain comprises or consists essentially of or consists of an amino acid sequence selected from the group consisting of
a) the sequence set forth in SEQ ID NO: 24 or 32 [IgG1-Fc_FEA];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 7 substitutions, such as at most 6 substitutions, at most 5, at most 4, at most 3, at most 2 substitutions or at most 1 substitution, compared to the amino acid sequence defined in a) or b).

38. The binding agent for use of any one of claims 14-37, wherein the constant region of said first and/or second heavy chain, such as the second heavy chain, comprises or consists essentially of or consists of an amino acid sequence selected from the group consisting of
a) the sequence set forth in SEQ ID NO: 25 or 33[IgG1-Fc_FEAL];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 6 substitutions, such as at most 5 substitutions, at most 4 substitutions, at most 3, at most 2 substitutions or at most 1 substitution, compared to the amino acid sequence defined in
a) or b).

39. The binding agent for use of any one of claims 14-38, wherein the constant region of said first and/or second heavy chain, such as the first heavy chain, comprises or consists essentially of or consists of an amino acid sequence selected from the group consisting of
a) the sequence set forth in SEQ ID NO: 26 or 34 [IgG1-Fc_FEAR];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 6 substitutions, such as at most 5 substitutions, at most 4, at most 3, at most 2 substitutions or at most 1 substitution, compared to the amino acid sequence defined in a) or b).

40. The binding agent for use of any one of the preceding claims, wherein said binding agent comprises a kappa (κ) light chain constant region.

41. The binding agent for use of any one of the preceding claims, wherein said binding agent comprises a lambda (λ) light chain constant region.

42. The binding agent for use of any one of the preceding claims, wherein said first light chain constant region is a kappa (κ) light chain constant region or a lambda (λ) light chain constant region.

43. The binding agent for use of any one of the preceding claims, wherein said second light chain constant region is a lambda (λ) light chain constant region or a kappa (κ) light chain constant region.

44. The binding agent for use of any one of the preceding claims, wherein said first light chain constant region is a kappa (κ) light chain constant region and said second light chain constant region is a lambda (λ) light chain constant region or said first light chain constant region is a lambda (λ) light chain constant region and said second light chain constant region is a kappa (κ) light chain constant region.

45. The binding agent for use of any one of claims 40-44, wherein the kappa (κ) light chain comprises an amino acid sequence selected from the group consisting of
a) the sequence set forth in SEQ ID NO: 27,
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).

46. The binding agent for use of any one of claims 41-45, wherein the lambda (λ) light chain comprises an amino acid sequence selected from the group consisting of
a) the sequence set forth in SEQ ID NO: 28,
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).

47. The binding agent for use of any one of the preceding claims, wherein the binding agent is of an isotype selected from the group consisting of IgG1, IgG2, IgG3, and IgG4.

48. The binding agent for use of any one of the preceding claims, wherein the binding agent is a full-length IgG1 antibody.

49. The binding agent for use of any one of the preceding claims, wherein the binding agent is an antibody of the IgG1m(f) allotype.

50. The binding agent for use of any one of the preceding claims, wherein the subject is a human subject.

51. The binding agent for use of any one of the preceding claims, wherein the tumor or cancer is a solid tumor.

52. The binding agent for use of any one of the preceding claims, wherein the tumor or cancer is selected from the group consisting of melanoma, ovarian cancer, lung cancer (e.g., non-small cell lung cancer (NSCLC)), colorectal cancer, head and neck cancer, gastric cancer, breast cancer, renal cancer, urothelial cancer, bladder cancer, esophageal cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, ovarian cancer, endometrial cancer, prostate cancer, penile cancer, cervical cancer, Hodgkin's lymphoma, non-Hodgkin's lymphoma, Merkel cell carcinoma and mesothelioma.

53. The binding agent for use of any one of the preceding claims, wherein the tumor or cancer is selected from the group consisting of lung cancer (e.g., non-small cell lung cancer (NSCLC)), melanoma, colorectal cancer, urothelial cancer (cancer of the bladder, ureter, urethra, or renal pelvis), endometrial cancer (EC), breast cancer (e.g., triple negative breast cancer (TNBC)), squamous cell carcinoma of the head and neck (SCCHN) (e.g., cancer of the oral cavity, pharynx or larynx) and cervical cancer.

54. The binding agent for use of any one of the preceding claims, wherein the tumor or cancer is selected from the group consisting of lung cancer, melanoma, and colorectal cancer.

55. The binding agent for use of claim 54, wherein the cancer is a non-small cell lung cancer (NSCLC), such as a squamous or non-squamous NSCLC.

56. The binding agent for use of claim 55, wherein the NSCLC does not have an epidermal growth factor (EGFR)-sensitizing mutation and/or anaplastic lymphoma (ALK) translocation/ROS1 rearrangement.

57. The binding agent for use of claim 55 or 56, wherein the subject has received up to four prior systemic treatment regimens for advanced/metastatic disease and has experienced disease progression on or after last prior systemic treatment, such as disease progression determined by radiography.

58. The binding agent for use of claim 57, wherein the subject has received platinum-based chemotherapy.

59. The binding agent for use of claim 57, wherein the subject is not eligible for platinum-based therapy and has alternative chemotherapy, e.g., a treatment with gemcitabine-containing regimen.

60. The binding agent for use of any one of claims 55-59, wherein the subject has received prior treatment with checkpoint inhibitor(s), such as agent(s) targeting PD-1/PD-L, such as a PD-1/PD-L1 inhibitor.

61. The binding agent for use of any one of claim 55-60, wherein the subject has experienced disease progression on or after last prior systemic treatment, such as disease progression determined by radiography.

62. The binding agent for use of claim 54, wherein the cancer is cutaneous, acral, or mucosal melanoma.

63. The binding agent for use of claim 62, wherein the subject has received up to four prior systemic treatments for advanced/metastatic disease and has experienced disease progression on or after last prior systemic treatment, such as disease progression determined by radiography.

64. The binding agent for use of claim 62 or 63, wherein the subject has received prior treatment with checkpoint inhibitor(s), such as agent(s) targeting PD-1/PD-L, such as a PD-1/PD-L1 inhibitor.

65. The binding agent for use of claim 54, wherein the cancer is cancer is colorectal cancer.

66. The binding agent for use of claim 65, wherein the subject has received up to four prior systemic treatments for advanced/metastatic disease and has experienced disease progression on or after last prior systemic treatment, such as disease progression determined by radiography.

67. The binding agent for use of claim 65 or 66, wherein the subject has received 5-FU-based therapy.

68. The binding agent for use of any one of claims 65-67, wherein the subject has not received treatment with an ICP inhibitor.

69. The binding agent for use of any one of the preceding claims, wherein the binding agent is administered in at least one treatment cycle, each treatment cycle being three weeks (21 days).

70. The binding agent for use of any one of the preceding claims, wherein one dose is administered every third week (1Q3W).

71. The binding agent for use of any one of the preceding claims, wherein one dose is administered on day 1 of each treatment cycle.

72. The binding agent for use of any one of the preceding claims, wherein each dose is infused over a minimum of 30 minutes, such as over a minimum of 60 minutes, a minimum of 90 minutes, a minimum of 120 minutes or a minimum of 240 minutes.

73. A composition comprising a binding agent comprising a first binding region binding to human CD40 and a second binding region binding to human CD137, wherein the amount of binding agent in the composition is between about 3-200 mg or about 20×10⁻⁹-1350×10⁻⁹ mol.

74. The composition according to claim 73, comprising about 40 mg of said binding agent.

75. The composition according to claim 73 or 74, wherein the binding agent is as defined in any one of claims 1-72.

76. The composition according to any one of claims 73-75, wherein the composition is for systemic administration.

77. The composition according to any one of claims 73-76, wherein the composition is for injection or infusion, such as intravenous injection or infusion.

78. The composition according to any one of claims 73-77, wherein the binding agent is in aqueous solution, such in 0.9% NaCl (saline), at a volume of 50-500 ml, such as 100-250 ml.

79. The composition according to any one of claims 73-78, said composition being a dosage unit form.

80. The composition according to any one of claims 73-79 for use in a method for reducing or preventing progression of a tumor or treating cancer in a subject.
